(19) United States
(12) Patent Application Publication
(10) Pub. No.: US 2024/0299561 A1
(43) Pub. Date: Sep. 12, 2024

(54) HISTIDINE-SELECTIVE POLYMER REAGENTS

(71) Applicant: Nektar Therapeutics, San Francisco, CA (US)

(72) Inventors: Antoni Kozlowski, Huntsville, AL (US); Neel K. Anand, San Mateo, CA (US); Xiaoming Shen, San Diego, CA (US); Xiaobing Wang, Redmond, WA (US)

(21) Appl. No.: 18/570,610

(22) PCT Filed: Jun. 17, 2022
(86) PCT No.: PCT/US2022/034085

§ 371 Date: Dec. 14, 2023

Related U.S. Application Data
(60) Provisional application No. 63/211,853, filed on Jun. 17, 2021.

(30) Foreign Application Priority Data
Jun. 17, 2021 (US) 63/211853

(51) Int. Cl. A61K 47/60; C08G 65/333

(52) U.S. Cl. CPC A61K 47/60; C08G 65/3332

(57) ABSTRACT

The instant disclosure provides (among other things) novel water-soluble polymer reagents capable of selective conjugation to histidine residues. e.g., in peptides and proteins, as well the resulting conjugates and related compositions. In addition, provided are methods of preparing the polymer reagents, as well as methods for conjugating the polymer reagents to active agents and other substances, pharmaceutical compositions, and methods for administering the conjugates.

### CROSS REFERENCE TO RELATED APPLICATIONS

This application claims the benefit of priority to U.S. Provisional Patent Application No. 63/211,853, filed Jun. 17, 2021, the disclosure of which is incorporated herein by reference in its entirety.

### FIELD

The instant application relates to (among other things) novel water-soluble polymer reagents capable of selective conjugation to histidine residues, e.g., in peptides and proteins, as well as to the conjugates formed by reaction with such reagents. In addition, provided are methods of preparing the polymer reagents, as well as methods for conjugating the polymer reagents to active agents and other substances, pharmaceutical compositions, and methods for administering the conjugates.

### BACKGROUND

Modification of bioactive molecules by covalent attachment of polyethylene glycol, often referred to a “PEGylation”, can be effective in providing new and differentiated therapeutic products. PEGylation can, for example, enhance the pharmacological and pharmaceutical properties of a bioactive molecule, and has been used successfully in the development of several marketed drug products. There are currently more than ten different PEGylated protein therapeutics that have been approved by the FDA. For example, PEGylation has been used to create marketed products in which a biopharmaceutical agent is covalently attached to polyethylene glycol with a stable bond, such as, for example, ClMZIA® (PEGylated tumor necrosis factor (TNF)), NEULASTA® (PEGylated granulocyte-colony stimulating factor (GCSF)), PEGASYS® (PEGylated interferon α-2a), and ADYNOVATE® (PEGylated Factor VIII). Most of these products rely on non-specific PEGylation of the proteins by reaction with amine groups of lysine residues and the N-terminus.

In many cases, stable covalent attachment of one or more polyethylene glycol chains to an active agent results in PEG conjugates having reduced functional activity when compared to the unmodified molecule. When an active molecule is covalently attached to one or more polymer moieties via a stable linkage, the polymer-bound active molecule may or may not retain the functional properties of the unbound active molecule, since the covalently attached polymer can change, among other things, the steric and electronic environment surrounding the active molecule. Often, non-selective PEGylation by reaction with amino groups of lysine residues and the N-terminus results in a heterogeneous mixture of PEGylated molecules, wherein each PEG conjugate within the mixture may possess a different or altered biological activity or other functional biological property.

In an effort to circumvent some of the issues noted above, site-specific PEGylation has been explored, although few PEGylation strategies exist that achieve site-specific conjugation of PEG to a protein or peptide. Among other advantages, site-specific PEGylation approaches may have a better probability of providing a well-defined and therapeutically useful PEGylated product, generally a single mono-PEGylated product, that is typically easier to purify, characterize, and prepare in a reproducible fashion.

For example, site-specific PEGylation can be afforded by covalent attachment to a cysteine, or more particularly, the thiol group of a cysteine. Cysteine residues are not an abundantly occurring amino acid in proteins, and account for less than one percent of the total amino acid content of proteins. Moreover, cysteines that do occur in proteins often form disulfide bonds, thus making them unavailable for reaction with many thiol-specific PEGylation reagents. To circumvent the lack or unavailability of cysteines in a protein candidate for PEGylation, cysteine-muteins can be prepared by genetically encoding one or more cysteine residues into specific locations in a protein. Reaction of a cysteine mutein with a thiol-selective or thiol-specific PEGylation reagent may then be carried out to prepare a protein with a PEG moiety covalently attached at the particular cysteine insertion site(s). PEG reagents suitable for reaction with cysteines include those with reactive groups such as thiol, disulfide, maleimide, vinyl sulfone, orthopyridyl disulfide, and iodoacetamide. One commonly employed approach for cysteine-directed site-specific PEGylation involves reaction of a cysteine-mutein or cysteine-containing protein with a maleimide-functionalized PEG reagent.

In another approach for achieving site-specific PEGylation, polyhistidine tags have been employed (Cong, Y., et al., Bioconjugate Chem. 2012, 23-248-263) to form His-tag specific PEGylated proteins including a domain antibody (dAb) that binds tumor-necrosis factor alpha and interferon α-2a (IFN). A 6-histidine tag was added to the C-terminus of dAb, while a 8-histidine tag was inserted on the N-terminal of IFN, followed by reaction with a PEG-bis-sulfone reagent capable of site-specific PEGylation by bis-alkylation following elimination of one equivalent of sulfinic acid to form the corresponding PEG-mono-sulfones.

Both of above-described approaches require either the insertion of a cysteine residue (unless a reactive cysteine is present in the unmodified protein) or the insertion of a polyhistidine sequence into a peptide or protein sequence of interest.

Like cysteines, histidines are of relatively low occurrence (˜2%) in globular proteins, and only about half of histidines are surface-accessible, making histidines an attractive target for site-specific PEGylation. Moreover, in contrast to cysteine residues, histidines, like lysines, provide a reactive amino group for amine-directed PEGylation within the imidazole ring. While histidine-directed conjugates have been previously prepared, these conjugates were found to be unstable, and reported to undergo hydrolysis in aqueous buffers under physiological conditions. See, e.g., Veronese, F. M., et al., U.S. Patent Publication No. US 2009/0185998; Lee, S., McNemar, C., U.S. Pat. No. 5,985,263. To the Applicant's knowledge, heretofore, PEG reagents capable of forming stably linked (e.g., non-releasable) conjugates by covalent attachment to an amino group of a histidine residue, e.g., of a protein or peptide, and the resulting conjugates, have been unknown in the art.

The instant disclosure provides, among other things, novel PEG reagents capable of site-selective modification of a histidine, e.g., such as in a peptide or protein. The resulting conjugates are stable over a broad range of pHs, including physiological pH, thereby allowing facile chromatographic purification, and the provision of, for example, a reproducibly prepared and well-defined, homogeneous PEGylated biopharmaceutical product having consistent and advantageous pharmacokinetic and pharmacodynamic properties and ideally, improved bioactivity when compared to a biopharmaceutical having one or more PEG moieties covalently attached in a non-selective/non-specific manner. To the Applicants' knowledge, the presently described polymers, conjugates, compositions and methods are novel and completely unsuggested by the art.

### SUMMARY

In a first aspect, provided is a polymer reagent of Formula I:

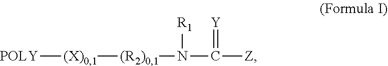

wherein POLY is a water-soluble polymer; X is a linker moiety; R₁ is an organic radical and may form a nitrogen-containing heterocycle when taken together with R₂; R₂, when present, taken together with R₁ forms a nitrogen-containing heterocycle; Y is either O (oxygen) or S (sulfur); and Z is a leaving group. The polymer reagent can be used for site-selective modification of a histidine, e.g., such as in a peptide or protein.

In some embodiments, R₁ is an organic radical selected from substituted and unsubstituted alkyl, substituted and unsubstituted cycloalkyl, substituted and unsubstituted alkenyl, substituted and unsubstituted cycloalkenyl, substituted and unsubstituted alkynyl, substituted and unsubstituted heteroalkyl, substituted and unsubstituted cycloheteroalkyl, substituted and unsubstituted aryl, substituted and unsubstituted aralkyl, substituted and unsubstituted heteroaryl, and substituted and unsubstituted heteroaralkyl.

In some further embodiments, R₁ is an organic radical selected from alkyl, cycloalkyl, alkenyl, cycloalkenyl, alkynyl, heteroalkyl, cycloheteroalkyl, aryl, aralkyl, heteroaryl, and heteroaralkyl, each optionally substituted with one or more substituents independently selected from the group consisting of halo, alkylhalo, hydroxy, alkylhydroxy, alkoxy, amino, alkylamino, sulfhydryl, alkylsulfhydryl, nitro, alkylnitro, cyano, alkylcyano, thiocyano, alkylthiocyano, imino, alkylimino, carbamate, alkylcarbamate, phosphate, alkylphosphate, alkylcarbonyl, carboxamide, alkylcarboxamide, alkoxycarbonyl, thioalkyl, thioester, and alkylthioester.

In yet one or more particular embodiments, Riis selected from the group consisting of lower alkyl, halo-substituted lower alkyl, benzyl, halo-substituted benzyl and nitro-substituted benzyl, wherein a benzyl ring has from one to five halo-substituents. In some embodiments related to the foregoing, the halo substituent is fluoro.

In one or more embodiments of Formula (I), R₂ is absent.

In yet some other embodiments of Formula (I), R₂ is present. When R₂ is present, in some embodiments, R₂, taken together with R₁, forms a nitrogen-containing heterocycle containing 4, 5, 6, or 7 heterocycle ring atoms, such as for example, a non-aromatic, saturated, nitrogen-containing heterocycle. In some other embodiments, when R₂ is present, the nitrogen-containing heterocycle contains from one to three nitrogen atoms (e.g., one, two, or three nitrogen atoms). Illustrative nitrogen-containing heterocycles include, for example, azetidine, substituted azetidine, diazetidine, substituted diazetidine, pyrrolidine, substituted pyrrolidine, imidazolidine, substituted imidazolidine, piperidine, substituted piperidine, morpholine, substituted morpholine, diazinanes, substituted diazinanes, triazinanes, substituted triazinanes, azepanes, substituted azepanes, diazepanes and substituted diazepanes. In some particular embodiments, R₂ together with R₁ forms a piperidine or a substituted piperidine. In some other embodiments, R₂ taken together with R₁ forms a diazinane or a substituted diazinane. In yet some additional particular embodiments, the diazinane or substituted diazinane is piperazine or a substituted piperazine, respectively.

In reference to embodiments in which R₂ taken together with R₁ forms a nitrogen-containing heterocycle, in some embodiments, the nitrogen-containing heterocycle is optionally substituted with one or more substituents such as, for example, alkyl, substituted alkyl, alkenyl, substituted alkenyl, alkynyl, substituted alkynyl, heteroalkyl, substituted heteroalkyl, aralkyl, substituted aralkyl, halo, alkylhalo, hydroxy, alkylhydroxy, alkoxy, amino, alkylamino, sulfhydryl, alkylsulfhydryl, nitro, alkylnitro, cyano, alkylcyano, thiocyano, alkylthiocyano, imino, alkylimino, carbamate, alkylcarbamate, phosphate, alkylphosphate, alkylcarbonyl, carboxamide, alkylcarboxamide, alkoxycarbonyl, thioalkyl, thioester, and alkylthioester.

In some more particular embodiments, R₂ taken together with R₁ forms a nitrogen-containing heterocycle that is (i) unsubstituted or (ii) is substituted at one or more ring positions with lower alkyl, substituted lower alkyl, aralkyl, or substituted aralkyl. In yet some further embodiments, a substituted lower alkyl or substituted aralkyl substituent is halo-substituted.

In yet one or more further embodiments, R₂ taken together with R₁ forms a nitrogen-containing heterocycle that is mono- or di-substituted.

In some embodiments of Formula (I), X is absent (i.e., is (X)o). In yet some other embodiments, X is present (i.e., is (X)₁). In some embodiments, X is selected from —O—, —S—, —NH——C(O)—, —O—C(O)—, —C(O)—O—, —C(O)—NH—, —NH—C(O)—NH—, —O—C(O)—NH—, —C(S)—, —CH₂—, —CH₂-CH₂—, —CH₂—CH₂—CH₂—, —CH₂—CH₂—CH₂—CH₂, —O—CH₂—, —CH₂—O—, —O—CH₂—CH₂—, —CH₂—O—CH 2—, —CH₂—CH₂—O—, —O—CH₂—CH₂—CH₂—, —CH₂—O—CH₂—CH₂—, —CH₂—CH₂—O—CH₂—, —CH₂—CH₂—CH₂—O—, —O—CH₂—CH₂—CH₂—CH₂—, —CH₂—O—CH₂—CH₂—CH₂—, —CH₂—CH₂—O—CH₂—CH₂—, —CH₂—CH₂—CH₂—O—CH₂—, —CH₂—CH₂—CH₂—CH₂—O—, —C(O)—NH—CH₂—, —C(O)—NH—CH₂—CH₂—, —CH₂—C(O)—NH—CH₂—, —CH₂-CH₂—C(O)—NH—, —C(O)—NH—CH₂—CH₂—CH₂—, —CH₂—C(O)—NH—CH₂—CH₂—, —CH₂—CH₂—C(O)—NH—CH₂—, —CH₂—CH₂—CH₂—C(O)—NH—, —C(O)—NH—CH₂—CH₂—CH₂—CH₂—, —CH₂—C(O)—NH—CH₂—CH₂—CH₂—, —CH₂—CH₂—C(O)—NH—CH₂—CH₂—, —CH₂—CH₂—CH₂—C(O)—NH—CH₂—, —CH₂—CH₂—CH₂—C(O)—NH—CH₂—CH₂—, —CH₂—CH₂—CH₂—CH₂—C(O)—NH—, —C(O)—O—CH₂—, —CH₂—C(O)—O—CH₂—, —CH₂—CH₂C(O)—O—CH₂—, —C(O)—O—CH₂—CH₂—, —NH—C(O)—CH₂—, —CH₂—NH—C(O)—CH₂—, —CH₂—CH₂—NH—C(O)—CH₂—, —NH—C(O)—CH₂—CH₂—, —CH₂—NH—C(O)—CH₂—CH₂—, —CH₂—CH₂—NH—C(O)—CH₂—CH₂—, —C(O)—NH—CH₂—, —C(O)—NH—CH₂—CH₂—, —O—C(O)—NH—CH₂—, —O—C(O)—NH—CH₂CH₂, —O—C(O)—NH—CH₂—CH₂—CH₂—, —NH—CH₂—, —NH—CH₂—CH₂—, —CH₂—NH—CH₂—, —CH₂—CH₂—NH—CH₂—, C(O)CH₂—, —C(O)—CH₂—CH₂—, —CH₂—C(O)—CH₂—, —CH₂—CH₂C(O)—CH₂—, —CH₂—CH₂—C(O)—CH₂—CH₂—, —CH₂—CH₂—C(O)—, —CH₂—CH₂—CH₂—C(O)—NH—CH₂—CH₂—NH—, —CH₂—CH₂—CH₂—C(O)—NH—CH₂—CH₂—NH—C(O)—, —CH₂—CH₂—CH₂—C(O)—NH—CH₂—CH₂—NH—C(O)—CH₂—, —CH₂—CH₂—CH₂—C(O)—NH—CH₂—CH₂—NH—C(O)—CH₂—CH₂—, —O—C(O)—NH—(CH₂)₀₋₆—(OCH₂CH₂)₀₋₂—, C(O)—NH—(CH₂)₁₋₆—NH—C(O)—, —NH—C(O)—NH—(C H₂)₁₋₆—NH—C(O)—, —O—C(O)—CH₂—, —O—C(O)—CH₂—CH₂—, —O—C(O)—CH₂—CH₂—CH₂—, and combinations of any one or more of the foregoing. In some further embodiments, X is ˜(CH₂)ₐ(O)_{b}[C(O)]_{c}(NH)_{d}(CH₂)ₑ˜, wherein: a is 0-6; bis 0,1; c is 0,1; dis 0,1; and e is 0-6, wherein at least one of a, b, c, d, and e is a positive integer. In some other embodiments, X is —O—C(O)—, —O—C(O)—NH— or —O—C(O)—NH—CH₂—.

In one or more embodiments, Y is O (oxygen).

In yet some other embodiments, Y is S (sulfur).

The water-soluble polymer, POLY, in some embodiments, is selected from poly(alkylene oxide), poly(vinyl pyrrolidone), poly(vinyl alcohol), polyoxazoline, poly(acryloylmorpholine), polyacrylic acid, polyacrylamides, N-(2-hydroxypropyl) methylacrylamide, divyinyl ether-maleic anhydride, polyphosphates, polyphosphazenes, and co-polymers and ter-polymers thereof. In some embodiments, POLY is a water-soluble poly(alkylene oxide). In some preferred embodiments, POLY is a poly(ethylene glycol). In some other preferred embodiments, the poly(ethylene glycol) is terminally capped with an end-capping moiety, such as, for example, hydroxy, alkoxy, substituted alkoxy, alkenoxy, substituted alkenoxy, alkynoxy, substituted alkynoxy, aryloxy or substituted aryloxy. In some preferred embodiments, the poly(ethylene glycol) is end-capped with a lower alkyl group, such as, for example, methoxy.

In one or more embodiments, POLY is a water-soluble polymer that is linear, branched, or multi-armed. For example, in some embodiments, POLY is a linear water-soluble polymer. In other embodiments, POLY is a branched water-soluble polymer. In yet some further embodiments, POLY is a multi-armed water-soluble polymer.

In some embodiments, POLY has a weight average molecular weight from about 100 daltons to about 100,000 daltons.

In one or more additional embodiments, POLY has a weight average molecular weight in a range of from about 200 daltons to about 80,000 daltons, or from about 500 daltons to about 70,000 daltons, or from about 1,000 daltons to about 60,000 daltons, or from about 5,000 daltons to about 25,000 daltons, or from about 5,000 daltons to about 30,000 daltons, or from about 5,000 daltons to about 50,000 daltons, or from about 10,000 daltons to about 60,000 daltons, or from about 10,000 daltons to about 50,000 daltons, or from about 20,000 daltons to about 50,000 daltons, or from about 20,000 daltons to about 40,000 daltons, or from about 20,000 daltons to about 80,000 daltons.

In yet some further more particular embodiments, POLY has a weight average molecular weight of about 200 daltons, or about 300 daltons, or about 400 daltons, or about 500 daltons, or about 750 daltons, or about 1,000 daltons, or about 2,500 daltons, or about 3,000 daltons, or about 5,000 daltons, or about 7500 daltons, or about 10,000 daltons, or about 15,000 daltons, or about 20,000 daltons, or about 25,000 daltons, or about 30,000 daltons, or about 40,000 daltons, or about 50,000 daltons, or about 55,000 daltons, or about 60,000 daltons, or about 65,000 daltons, or about 70,000 daltons, or about 75,000 daltons, or greater than 75,000 daltons.

In one or more embodiments, Z is selected from the group consisting of tetrazoles, isocyanates, isothiocyanates, N-hydroxysuccinimide, acyl azide, fluorophenol, benzotriazoles, nitrophenols, and triazoles.

In one or more embodiments, Z is a leaving group that when taken with ˜N(R₁)C(Y)— forms a urea bond.

In some preferred embodiments, Z is a tetrazole leaving group. Illustrative tetrazole leaving groups include phenyl tetrazoles. For example, Z may be a phenyl tetrazole having a structure:

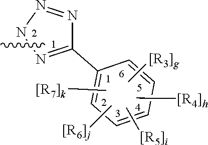

wherein g, h, i, j, and k is each independently 0 or 1 (wherein 0 indicates absence and 1 indicates presence), and each of R₃, R₄, R₅, R₆ and R₇ is independently selected from the group consisting of alkyl, substituted alkyl, alkenyl, substituted alkenyl, alkynyl, substituted alkynyl, heteroalkyl, substituted heteroalkyl, aralkyl, substituted aralkyl, halo, alkylhalo, hydroxy, alkylhydroxy, alkoxy, amino, alkylamino, sulfhydryl, alkylsulfhydryl, nitro, alkylnitro, cyano, alkylcyano, thiocyano, alkylthiocyano, imino, alkylimino, carbamate, alkylcarbamate, phosphate, alkylphosphate, alkylcarbonyl, carboxamide, alkylcarboxamide, alkoxycarbonyl, thioalkyl, thioester, and alkylthioester. In some embodiments, the phenyl tetrazole has a single substituent on the phenyl ring, at any one of carbon 2, carbon 3, or carbon 4. In yet some other embodiments, the phenyl tetrazole has two substituents on the phenyl ring. For example, the two phenyl substituents may be (i) at C2 and C3, or (ii) at C2 and C4, or (iii) at C3 and C5, or (iv) at C3 and C4, or at C2 and C6. In yet some further embodiments, the phenyl tetrazole has three substituents on the phenyl ring, e.g., (i) at C2, C3 and C4, or (ii) at C2, C3, and C5, or (iii) at C2, C3 and C6, or (iv) at C2, C4, and C6, or (v) at C3, C4, and C5, or (vi) at C3, C4, and C6. In yet some other embodiments, the phenyl tetrazole has four substituents on the phenyl ring, e.g., (i) at C2, C3, C4, and C5, or (ii) at C2, C3, C5, and C6, or (iii) at C2, C3, C4, and C6. In yet some other embodiments, the phenyl tetrazole has five substituents on the phenyl ring. In some embodiments related to any one or more of the foregoing wherein the phenyl tetrazole has more than one substituent on the phenyl ring, the substituents on the phenyl ring are the same. In yet some alternative embodiments, one or more of the substituents on the phenyl ring are different. In some embodiments, one or more of the substituents is trifluoromethyl. In some other particular embodiments, Z is a bis(trifluoromethyl)phenyl tetrazole.

In some further embodiments, the polymer reagent is selected from:

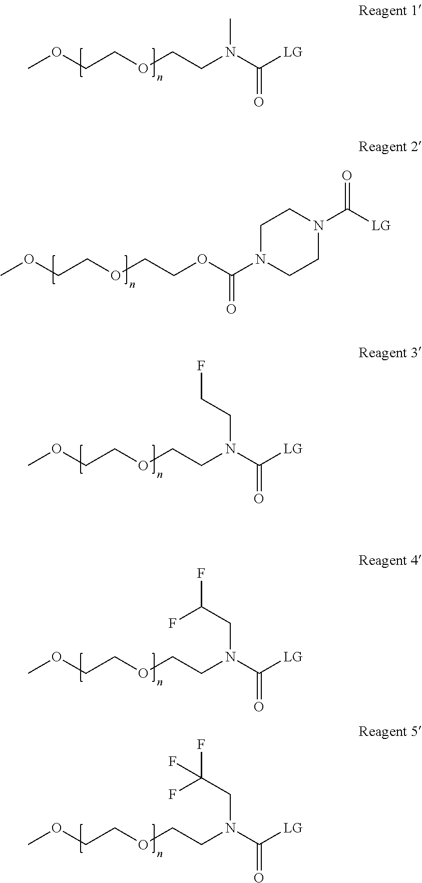

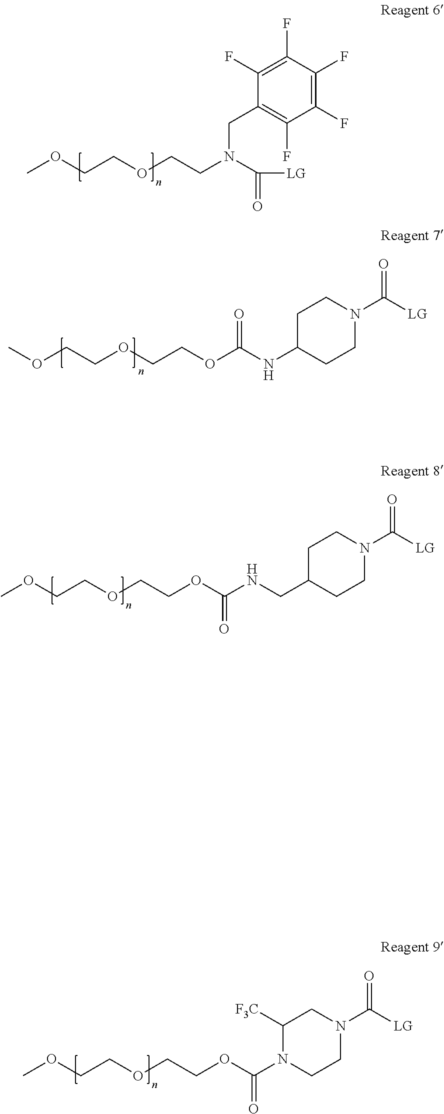

wherein each (n) is independently in a range selected from the group consisting of: from about 2 to about 2,273; from about 4 to about 1800; from about 11-1590; from about 23 to about 1363; from about 113 to about 568; from about 113 to about 682; from about 113 to about 1136; from about 227 to about 1363; from about 227 to about 1136; from about 454 to about 1136; from about 454 to about 909; and from about 454 to about 1818; and LG is a leaving group. In one or more embodiments related to Reagent 1′, 2′, 3′, 4′, 5′, 6′, 7′, 8′, and 9′, the LG is 3,5-bis(trifluoromethyl)phenyl-2H-tetrazole.

In still a further aspect, a method of preparing a polymer reagent, e.g., of Formula I, is provided.

In yet another aspect, provided is a water-soluble polymer conjugate of an active agent, wherein the linkage between the water-soluble polymer and the active agent comprises a urea-imidazolyl or a thiourea-imidazolyl moiety, and the imidazolyl group forms part of a histidine residue of the active agent.

In yet another aspect, provided herein is a conjugate prepared by reacting a polymer reagent of Formula (I), encompassing each and every of the embodiments provided above and disclosed herein, with an active agent comprising one or more amino groups under conditions effective to promote conjugation between the one or more amino groups of the active agent and the polymer reagent. In one or more embodiments related to such conjugate, the active agent is selected from a protein, a peptide, and a small molecule. In yet some other embodiments, the active agent comprises one or more histidine residues comprising an amino group (“histidine amino group”), and the one or more histidine amino groups are covalently attached to the ˜C(Y)˜ carbon of the polymer reagent.

In yet some other aspects, the conjugate is of a formula:

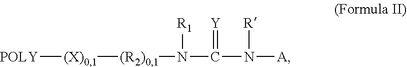

wherein POLY is a water-soluble polymer; X is a linker moiety; R₁ is an organic radical and may form a nitrogen-containing heterocycle when taken together with R₂; R₂, when present, taken together with R₁ forms a nitrogen-containing heterocycle; Y is selected from O and S; R′ is H or an organic radical, and A-N—R′ is an active agent (A) comprising an amino group (˜NR′), where POLY, X, R₂, R₁, and Y encompass each and every of the embodiments described above and further disclosed herein. In some preferred embodiments, ˜N-R′, when taken together with A, is an amine (nitrogen atom) comprised within an imidazolyl ring of a histidine comprised in the active agent.

In one or more embodiments, the conjugate has a formula:

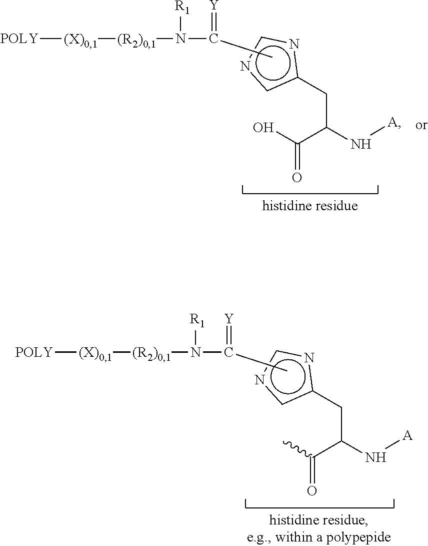

wherein A-NR′ is an active agent comprising a histidine residue, and POLY, X, R₂, R₁, and Y are as previously described. In the top formula, the histidine is shown as a single amino acid, while in the second structure, the histidine is depicted more particularly as being part of a polypeptide. However, it is to be understood that the histidine shown in the top structure, although drawn as a single amino acid, is intended to encompass its presence comprised in a peptide or polypeptide. It is to be understood that conjugation can take place at either nitrogen on the histidine imidazole ring, and the foregoing structures are intended to encompass both isomers. In some further preferred embodiments, the active agent is a peptide or a protein comprising a histidine residue.

Conjugates in accordance with the instant disclosure include, for example,

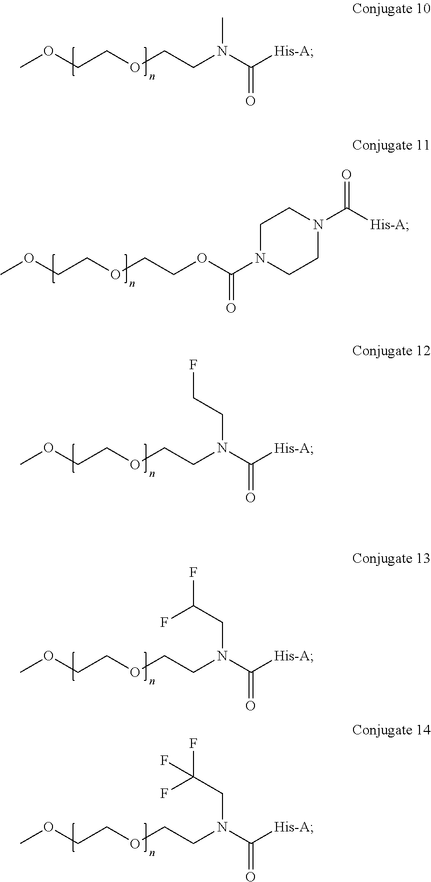

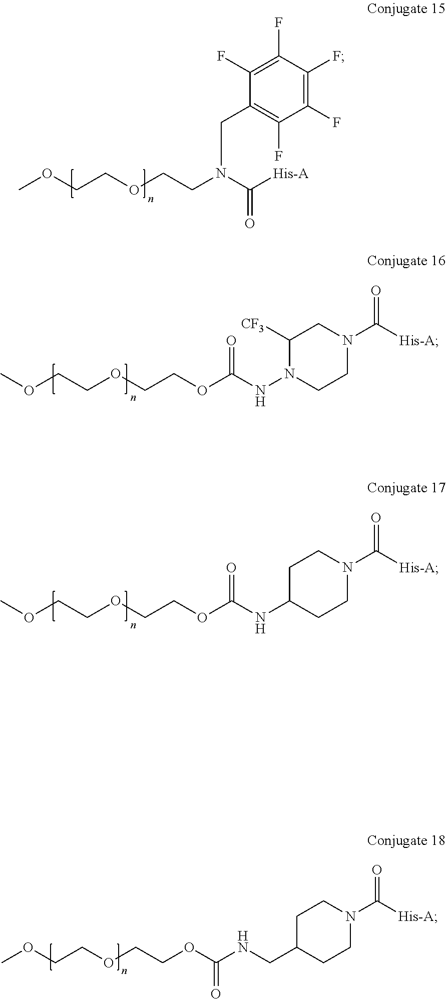

wherein (n) in each of Conjugates 11-18 is independently in a range of from about 2 to about 2,273 (including various embodiments thereof as described above and elsewhere herein); His is a histidine residue, wherein attachment is at a histidine nitrogen atom; and A-His is an active agent, such as for example, a peptide or protein, comprising a histidine residue.

In yet another aspect, provided herein is a pharmaceutical composition comprising a conjugate of Formula (II) (including embodiments thereof as set forth above and elsewhere herein) and a pharmaceutically acceptable excipient.

In yet another aspect, provided is a composition comprising conjugates in accordance with Formula (II) (including embodiments thereof as set forth above and elsewhere herein), wherein at least 60% of conjugates, or at least 75% of conjugates in the composition comprise POLY covalently attached to the active agent at only a histidine residue(s). In some further related embodiments, the composition further comprises a pharmaceutically acceptable excipient.

In yet a further aspect, provided is a method of preparing a conjugate of an active agent, the method comprising reacting a polymer reagent of Formula (I) (including each and every of the embodiments provided herein) with an active agent comprising one or more amino groups under conditions effective to promote conjugation between the one or more amino groups of the active agent and the polymer reagent. In some preferred embodiments, the active agent comprises one or more histidine residues comprising an amino group (“histidine amino group”) that reacts with the polymer reagent under the reaction conditions to thereby form a polymer conjugate.

Additional aspects and embodiments are set forth in the following description and claims.

### BRIEF DESCRIPTION OF THE DRAWINGS

FIG. 1 provides an illustrative reaction scheme for the synthesis of an exemplary histidine selective water-soluble polymer reagent, mPEG-N(CH₃)CO-5-(3,5-bis(triflu-oromethyl)phenyl)-2H-tetrazole (Reagent 1).

FIG. 2 is a table of illustrative histidine-selective poly(ethylene glycol) reagents containing a variety of spacer groups intervening between the PEG moiety and the BTMP-tetrazole (or other suitable) leaving group as indicated by the dashed boxes (i.e., ˜(X)_{0.1}—(R₂)_{0.1}—NR₁—C(O)˜) and their reactivities based upon percent conjugate formed under different pH conditions at a reaction time of five hours as described in Example 10.

FIG. 3 is a plot showing percent of histidine-linked conjugate formed over time at pH 5.0 at 25° C. for illustrative histidine-selective poly(ethylene glycol) reagents containing a variety of amino groups (X) intervening between the PEG moiety and the illustrative ˜C(O)BTMP-tetrazole (or other suitable) leaving group as described in Example 10.

FIG. 4 is a plot showing the results of a hydrolytic stability study for exemplary histidine-linked poly(ethylene glycol) conjugates containing a variety of spacer moieties intervening between the PEG moiety and the illustrative covalently-linked histidine of model compound, α-CBZ-histidine, as described in Example 14. Specifically, the plot demonstrates percent intact histidine-linked conjugate over time under physiological conditions (pH 7.4 at 37° C.).

FIG. 5 is a plot illustrating the reactivities of exemplary histidine-selective PEG reagents (as indicated by amino group, X, intervening between the PEG moiety and the illustrative ˜C(O)BTMP-tetrazole (or other suitable) leaving group) with carboxybenzyl (CBZ)-histidine at pH 5.5 at 25° C. as described in Example 10. Specifically, the plot shows percent of mPEG-histidine(α-CBZ) conjugate formed over time.

FIG. 6 is a plot illustrating the reactivities of exemplary histidine-selective PEG reagents (as indicated by amino group, X, intervening between the PEG moiety and the illustrative ˜C(O)BTMP-tetrazole (or other suitable) leaving group) with carboxybenzyl (CBZ)-histidine at pH 6.0 at 25° ° C. as described in Example 10. Specifically, the plot shows percent of mPEG-histidine(α-CBZ) conjugate formed over time.

FIG. 7 is a plot illustrating the reactivities of exemplary histidine-selective PEG reagents (as indicated by amino group, X, intervening between the PEG moiety and the illustrative ˜C(O)BTMP-tetrazole (or other suitable) leaving group) with carboxybenzyl (CBZ)-histidine at pH 6.5 at 25° C. as described in Example 10. Specifically, the plot shows percent of mPEG-histidine (α-CBZ) conjugate formed over time. As shown, by suitably modifying reaction conditions, e.g., in this case, by increasing pH), good yields of histidine-linked conjugates can be obtained.

FIG. 8 illustrates the reactivity of an illustrative histidine-selective PEG reagent, mPEG-4-aminopiperidine-C(O)-5-(3,5-bis(trifluoromethyl)phenyl-2H-tetrazole, 5 kD, with the model compound, carboxybenzyl (CBZ)-histidine, at four different pHs (5.0, 5.5, 6.0, and 6.5). The plot shows percent of mPEG-4-aminopiperidine-C(O)-histidine(α-CBZ) conjugate formed over time at each of the different pHs as described in Example 10. As shown, reactivity can be altered by, for example, changing the pH. As shown in the figure, after 10 hours, at pHs 5.0, 5.5, 6.0, and 6.5, the percent (%) conjugate formed was 4.7, 15, 57 and 93, respectively.

FIG. 9 illustrates the reactivity of one illustrative histidine-selective PEG reagent, mPEG-4-aminomethylpiperidine-C(O)-5-(3,5-bis(trifluoromethyl)phenyl-2H-tetrazole, 5 kD, with the model compound, carboxybenzyl (CBZ)-histidine, at four different pHs (5.0, 5.5, 6.0, and 6.5) as described in Example 10. The plot shows percent of mPEG-4-aminomethylpiperidine-C(O)-histidine(α-CBZ) conjugate formed over time at each of the different pHs. As shown, reactivity can be altered by, for example, changing the pH. As shown in the figure, after 10 hours, at pHs 5.0, 5.5, 6.0, and 6.5, the percent conjugate formed was 2.0, 7, 30 and 68, respectively. These data also illustrate the lower relative reactivity of the mPEG-4-aminomethylpiperidine-C(O)-5-(3,5-bis(trifluoromethyl)phenyl-2H-tetrazole reagent in comparison to the mPEG-4-aminopiperidine-C(O)-5-(3,5-bis(trifluoromethyl)phenyl-2H-tetrazole reagent, wherein the two reagents differ in the absence or presence of a ˜CH₂˜ group interposed between the carbamate nitrogen and the 4-carbon of the piperidine ring.

FIG. 10 provides reaction schemes for the conjugation of an exemplary PEG reagent, Reagent 2 (mPEG-piperazine-CO-5-(3,5-bis(trifluoromethyl)phenyl)-2H-tetrazole), with model compounds, α-CBZ-His, α-CBZ-Lys, and ω-CBZ-Lys-Gly-Gly-OH at a molar ratio of 1:10, in phosphate buffer at 25° C. The reactions illustrate the histidine-selectivity of the reagent, as supported by the data in FIG. 11.

FIG. 11 is a plot demonstrating the selectivity of an illustrative PEG reagent as provided herein, Reagent 2, where R₂ when taken with —NR₁ forms piperazine, and where ˜(X)_{0.1}—(R₂)_{0.1}—NR₁—C(O)˜) is ˜O—C(O)-piperazine-C(O)˜, when reacted with different amino acid or oligopeptide targets: α-CBZ-His, α-CBZ-Lys, and ω-CBZ-Lys-Gly-Gly-OH at a molar ratio of 1:10, in phosphate buffer at 25° C. The results illustrate the striking selectivity of the reagents provided herein for histidine over lysine as described in Example 11.

FIG. 12 is a plot demonstrating the selectivity of an illustrative PEG reagent as provided herein, Reagent 9, where R₂ when taken with —NR₁ forms piperazine, and where ˜(X)_{0.1}—(R₂)_{0.1}—NR₁—C(O)˜) is ˜O—C(O)-piperazine-C(O)˜, when reacted with different amino acid or oligopeptide targets: α-CBZ-His, α-CBZ-Lys, and ω-CBZ-Lys-Gly-Gly-OH at a molar ratio of 1:10, in phosphate buffer at 25° C. The results illustrate the striking selectivity of the reagents provided herein for histidine over lysine as described in Example 13.

FIG. 13 is a plot demonstrating the selectivity of an illustrative PEG reagent as provided herein, Reagent 7, where R₂ when taken with —NR₁ forms piperidine, and where ˜(X)_{0.1}—(R₂)_{0.1}—NR₁—C(O)˜) is ˜O—C(O)-NH-piperidine-C(O)˜, when reacted with different amino acid or oligopeptide targets: α-CBZ-His, α-CBZ-Lys, and ω-CBZ-Lys-Gly-Gly-OH at a molar ratio of 1:10, in phosphate buffer at 25° C. The results illustrate the striking selectivity of the reagents provided herein for histidine over lysine as described in Example 13.

FIGS. 14A and 14B provide further evidence of the histidine-selectivity of the polymer reagents provided herein as described in Example 15. More particularly, FIG. 14A is a plot illustrating reaction of a histidine conjugate prepared by reaction of Reagent 1 with model compound, α-CBZ-His, with hydroxylamine (pH 7.4, 25° C.). The plot shows percent of mPEG-N(CH₃)—CO-His(α-CBZ) conjugate remaining over time. Histidine conjugates such as the illustrative conjugates tested, when treated with hydroxylamine, undergo a reverse reaction such that the unconjugated histidine compound is released. In contrast, hydroxylamine does not typically react with a lysine linked conjugate to cleave the lysine-polymer linkage to thereby release the parent lysine compound. By approximately 22 hours, 100% of the conjugate has disappeared with release of α-CBZ-His. FIG. 14B is a plot illustrating reaction of a histidine conjugate prepared by reaction of Reagent 2 with model compound, α-CBZ-His, with hydroxylamine (pH 7.3, 25° C.). The plot shows percent of mPEG-piperazine-CO-His(α-CBZ) conjugate remaining over time. At approximately 42 hours, only 4% of the intact conjugate remained.

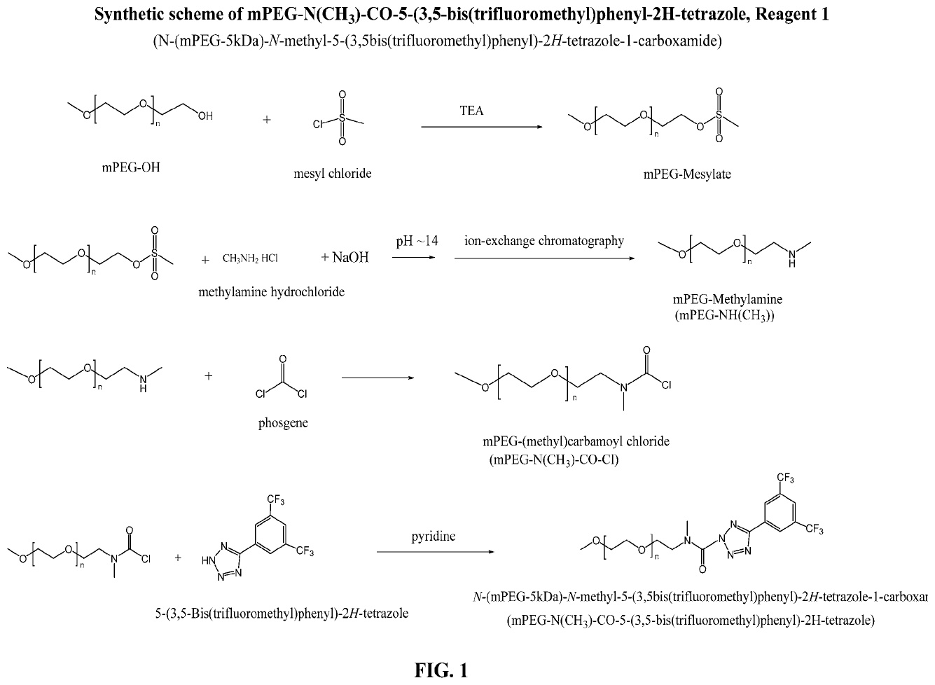

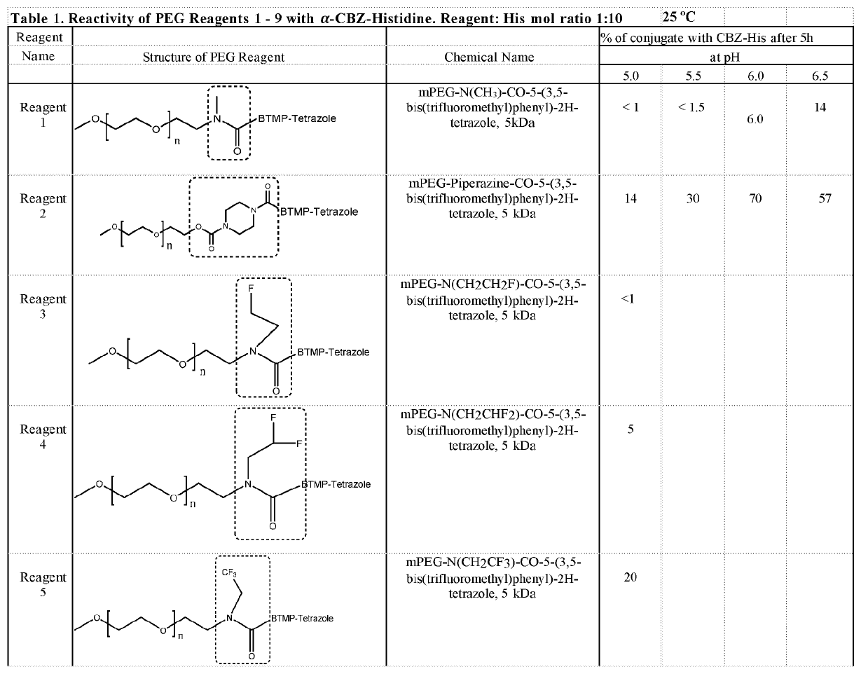

### DETAILED DESCRIPTION

### Definitions

In describing and claiming certain features of this disclosure, the following terminology will be used in accordance with the definitions described below unless indicated otherwise.

As used in this specification, the singular forms “a,” “an,” and “the” include plural referents unless the context clearly dictates otherwise. Thus, for example, reference to a “polymer” includes a single polymer as well as two or more of the same or different polymers, reference to a “conjugate” refers to a single conjugate as well as two or more of the same or different conjugates, reference to an “excipient” includes a single excipient as well as two or more of the same or different excipients, and the like.

“Water soluble, non-peptidic polymer” refers to a polymer that is at least 35% (by weight) soluble in water at room temperature. Preferred water soluble, non-peptidic polymers are however preferably greater than 70% (by weight), and more preferably greater than 95% (by weight) soluble in water. Typically, an unfiltered aqueous preparation of a “water-soluble” polymer transmits at least 75% of the amount of light transmitted by the same solution after filtering. Preferably, such unfiltered aqueous preparation transmits at least 95% of the amount of light transmitted by the same solution after filtering. Most preferred are water-soluble polymers that are at least 95% (by weight) soluble in water or completely soluble in water. With respect to being “non-peptidic,” a polymer is non-peptidic when it contains less than 35% (by weight) of amino acid residues.

The terms “monomer,” “monomeric subunit” and “monomeric unit” are used interchangeably herein and refer to one of the basic structural units of a polymer. In the case of a homo-polymer, a single repeating structural unit forms the polymer. In the case of a co-polymer, two or more structural units are repeated—either in a pattern or randomly—to form the polymer. Preferred polymers used in connection with the present invention are homo-polymers. The water-soluble, non-peptidic polymer comprises three or more monomers serially attached to form a chain of monomers.

“PEG” or “polyethylene glycol,” as used herein, is meant to encompass any water-soluble poly(ethylene oxide). Unless otherwise indicated, a “PEG polymer” or a polyethylene glycol is one in which substantially all (preferably all) monomeric subunits are ethylene oxide subunits, though, the polymer may contain distinct end capping moieties or functional groups, e.g., for conjugation. PEG polymers will generally comprise one of the two following structures: “—(CH₂CH₂O)ₙ-” or “—(CH₂CH₂O)ₙ-1CH₂CH₂—,” depending upon whether or not the terminal oxygen(s) has been displaced, e.g., during a synthetic transformation. As stated above, for the PEG polymers, the variable (n) ranges from about 2 to about 2273, and the terminal groups and architecture of the overall PEG can vary. Additional sub-ranges for “n” are described herein. PEG polymers in connection with the present disclosure are typically end-capped, where a preferred end-capping group is a lower alkyl group, with a most preferred end-capping group being methyl (also referred to as methoxy when considered with an adjacent oxygen atom).

In the context of the present disclosure, it should be recognized that the definition of a variable provided with respect to one structure or formula is applicable to the same variable repeated in a different structure, unless the context dictates otherwise. Thus, for example, the definitions of “POLY,” “a linker moiety (X),”, an organic radical (R₁) and so forth with respect to a polymeric reagent are equally applicable to a water-soluble polymer conjugate or a polymeric intermediate (as applicable) as provided herein.

Molecular weight in the context of a water-soluble polymer, such as PEG, can be expressed as either a number average molecular weight or a weight average molecular weight. Unless otherwise indicated, all references to molecular weight herein refer to the weight average molecular weight. Both molecular weight determinations, number average and weight average, can be measured using gel permeation chromatography or other liquid chromatography techniques (e.g. gel filtration chromatography). Most commonly employed are gel permeation chromatography and gel filtration chromatography. Other methods for determining molecular weight include end-group analysis or the measurement of colligative properties (e.g., freezing-point depression, boiling-point elevation, or osmotic pressure) to determine number average molecular weight or the use of light scattering techniques, ultracentrifugation, MALDI TOF, or viscometry to determine weight average molecular weight. PEG polymers are typically polydisperse (i.e., the number average molecular weight and the weight average molecular weight of the polymers are not equal), possessing low polydispersity values of preferably less than about 1.2, more preferably less than about 1.15, still more preferably less than about 1.10, yet still more preferably less than about 1.05, and most preferably less than about 1.03.

“Branched,” in reference to the geometry or overall structure of a polymer, refers to a polymer having two polymer “arms” or “chains” extending from a branch point or central structural feature. Examples of some preferred branched polymers are those having one or more of the following features: having two polymer arms comprised of polymer chains having the same structure (for example, comprised of the same monomer subunits), and comprised of polymer arms having the same average molecular weight.

“Multi-armed” in reference to the geometry or overall structure of a polymer refers to a polymer having 3 or more polymer-containing chains or “arms”. Thus, a multi-armed polymer may possess 3 polymer arms, 4 polymer arms, 5 polymer arms, 6 polymer arms, 7 polymer arms, 8 polymer arms or more, depending upon its configuration and core structure.

A “stable” linkage or bond refers to a chemical bond that is substantially stable in water, that is to say, does not undergo hydrolysis or degradation under physiological conditions to any appreciable extent over an extended period of time. Examples of hydrolytically stable linkages generally include but are not limited to the following: carbon-carbon bonds (e.g., in aliphatic chains), ether linkages, amide linkages, amine linkages, and the like. It is to be understood however, that the stability of any given chemical bond may be affected by the particular structural features of the molecule in which the bond is positioned as well as the placement of the subject linkage within a given molecule, adjacent and neighboring atoms, and the like, as will be understood by one of skill in the chemical arts. One of ordinary skill in the art can determine whether a given linkage is stable or releasable in a given context by, for example, placing a linkage-containing molecule of interest under conditions of interest (e.g., under physiological conditions) and testing for evidence of release over a suitable time period. Generally, a stable linkage is one that, for example, exhibits a rate of hydrolysis of less than about 1-2% per day under physiological conditions. Hydrolysis rates of representative chemical bonds can be found in most standard organic chemistry textbooks.

“Alkyl” refers to a hydrocarbon chain, typically ranging from about 1 to 15 atoms in length. Such hydrocarbon chains are preferably but not necessarily saturated and may be branched or straight chain, although typically straight chain is preferred. Exemplary alkyl groups include methyl, ethyl, propyl, butyl, pentyl, 3-methylpentyl, and the like.

“Lower alkyl” refers to an alkyl group containing from 1 to 6 carbon atoms, and may be straight chain or branched, as exemplified by methyl, ethyl, n-butyl, i-butyl, and t-butyl.

“Alkoxy” refers to an —OR group, wherein R is alkyl or substituted alkyl, preferably C₁₋₆ alkyl (e.g., methoxy, ethoxy, propyloxy, and so forth).

The term “substituted” as in, for example, “substituted alkyl,” refers to a moiety (e.g., an alkyl group) substituted with one or more noninterfering substituents, such as, but not limited to: alkyl, C₃₋₈ cycloalkyl, e.g., cyclopropyl, cyclobutyl, and the like; halo, e.g., fluoro, chloro, bromo, and iodo; cyano; alkoxy, lower phenyl; substituted phenyl; and the like. “Substituted aryl” is aryl having one or more noninterfering groups as a substituent. For substitutions on a phenyl ring, the substituents may be in any orientation (i.e., ortho, meta, or para). Substituents on aryl moieties that are a part of a more complex system, such as a naphthalene or fluorene core, may occupy any aryl ring position not otherwise occupied in the structure.

“Noninterfering substituents” are those groups that, when present in a molecule, are typically nonreactive with other functional groups contained within the molecule.

“Aryl” means one or more aromatic rings, each of 5 or 6 core carbon atoms. Aryl includes multiple aryl rings that may be fused, as in naphthyl or unfused, as in biphenyl. Aryl rings may also be fused or unfused with one or more cyclic hydrocarbon, heteroaryl, or heterocyclic rings. As used herein, “aryl” includes heteroaryl. An aromatic moiety (e.g., Ar¹, Ar², and so forth), means a structure containing aryl.

“Heteroaryl” is an aryl group containing from one to four heteroatoms, preferably sulfur, oxygen, or nitrogen, or a combination thereof. Heteroaryl rings may also be fused with one or more cyclic hydrocarbon, heterocyclic, aryl, or heteroaryl rings.

“Heterocycle” or “heterocyclic” means one or more rings of 5-12 atoms, preferably 5-7 atoms, with or without unsaturation or aromatic character and having at least one ring atom that is not a carbon. Preferred heteroatoms include sulfur, oxygen, and nitrogen.

“Substituted heteroaryl” is a heteroaryl having one or more noninterfering groups as substituents.

“Substituted heterocycle” is a heterocycle having one or more side chains formed from noninterfering substituents.

A “protecting group” is a moiety that prevents or blocks reaction of a particular chemically reactive functional group in a molecule under certain reaction conditions. The protecting group will vary depending upon the type of chemically reactive group being protected as well as the reaction conditions to be employed and the presence of additional reactive or protecting groups in the molecule. Functional groups which may be protected include, by way of example, carboxylic acid groups, amino groups, hydroxyl groups, thiol groups, carbonyl groups and the like. Representative protecting groups for carboxylic acids include esters (such as a p-methoxybenzyl ester), amides and hydrazides; for amino groups, carbamates (such as tert-butoxycarbonyl) and amides; for hydroxyl groups, ethers and esters; for thiol groups, thioethers and thioesters; for carbonyl groups, acetals and ketals; and the like. Such protecting groups are well-known to those skilled in the art and are described, for example, in T. W. Greene and G. M. Wuts, Protecting Groups in Organic Synthesis, Third Edition, Wiley, New York, 1999, and in Greene's Protective Groups in Organic Synthesis, Fifth Edition, John Wiley & Sons, Inc., New Jersey, 2014, and references cited therein.

A functional group in “protected form” refers to a functional group bearing a protecting group. As used herein, the term “functional group” or any synonym thereof, or reference to a particular functional group, is meant to encompass protected forms thereof as applicable.

Molecular entities as described herein, including for example, polymer reagents and their conjugates, are intended to encompass salt forms where applicable including pharmaceutically acceptable salts. See, for example, Stahl, P. H., Wermuth (Eds.), C. G., Handbook of Pharmaceutical Salts, Properties, Selection and Use, Wiley-VCH (Germany) and VHCA (Switzerland), 2002.

The term “linker” is used herein to refer to an atom or a collection of atoms used to link interconnecting moieties, such as, for example, a water-soluble polymer (POLY) and a nitrogen-containing heterocycle or a nitrogen atom (˜NR₁) as set forth in Formula (I). A linker (also referred to as a linker moiety) may be hydrolytically stable or may include a physiologically hydrolyzable or enzymatically degradable linkage. Preferably, as used herein, a linker is hydrolytically stable.

A “small molecule” may be defined broadly as an organic, inorganic, or organometallic compound typically having a molecular weight of less than about 1000 daltons.

An “organic radical” as used herein shall include alkyl, substituted alkyl, aryl, and substituted aryl.

“Substantially” or “essentially” means nearly totally or completely, for instance, 95% or greater of a given quantity.

Similarly, “about” or “approximately” as used herein means within plus or minus 5% of a given quantity.

“Optional” or “optionally” means that the subsequently described circumstance may but need not necessarily occur, so that the description includes instances where the circumstance occurs and instances where it does not.

“Pharmaceutically acceptable excipient” or “pharmaceutically acceptable carrier” refers to a component that may be included in the compositions described herein and causes no significant adverse toxicological effects to a subject.

The term “patient,” or “subject” as used herein refers to a living organism suffering from or prone to a condition that can be prevented or treated by administration of a compound or composition or combination as provided herein, such as a cancer, and includes both humans and animals. Subjects include, but are not limited to, mammals (e.g., murines, simians, equines, bovines, porcines, canines, felines, and the like), and preferably are human.

### Overview

The instant disclosure is directed to, among other things, novel PEG reagents capable of site-selective modification of a histidine, e.g., such as in a peptide or protein, as well as their corresponding conjugates. The new polymeric reagents can enhance the potential of water-soluble polymer-active agent conjugation by directing conjugation to histidine sites in an active molecule. This approach can offer significant advantages over PEGylation reactants that target protein amino groups on the side chains of lysines and the N-terminal, due to the abundance of available lysines in most protein therapeutics, which can lead to formation of a heterogenous mixture of conjugates having differing sites of PEG attachment and different numbers of PEG moieties attached to the protein. Histidine is a relatively rare amino acid in proteins; histidines have a lower pKa than other nucleophilic residues in a protein, such as, for example, lysine and arginine, such that reaction conditions for certain innovative reagents such as those provided herein can be tailored to favor histidine-selective conjugation. The discovery and design of water-soluble polymer reagents capable of selectively reacting at a histidine residue over lysine residues provides several notable advantages. For target proteins comprising a histidine residue, selective conjugation can be carried out without the need for protein engineering to introduce a desired conjugation site such as a cysteine or a non-natural amino acid, or alternatively, substitution of competing amino acids that undergo competitive conjugation, although such approaches are within the scope of this disclosure. Generally, it is expected that the polymeric reagents will form conjugated products comprising fewer positional isomers, as well as conjugated products having substantially the same number of PEG (or other water-soluble polymer) moieties attached to the active molecule. The corresponding conjugates, generally comprising an imidazolyl urea or thiourea linkage, are stable over a broad range of pHs, including physiological pH, thereby allowing facile chromatographic purification and handling, and the provision of, for example, a reproducibly prepared and well-defined, substantially homogeneous PEGylated biopharmaceutical product having consistent and advantageous pharmacokinetic and pharmacodynamic properties and ideally, improved bioactivity when compared to a biopharmaceutical stably covalently attached to multiple PEG moieties in a non-selective/non-specific manner.

These and other aspects and embodiments are described in greater detail in the sections which follow.

### Polymeric Reagents

Turning to a first aspect, unique polymeric reagents are provided. As disclosed previously, the polymeric reagents are capable of selective, that is, preferential, reaction with a histidine within a target molecule such as a biologically active molecule.

The polymeric reagent is described generally by the following formula (Formula I):

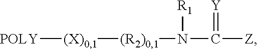

where POLY is a water-soluble polymer, X is a linker moiety (wherein the “0” subscript indicates its absence and the “1” subscript indicates its presence), R₁ is an organic radical and may, in some instances, form a nitrogen-containing heterocycle when taken together with R₂; R₂, when present (wherein the “0” subscript indicates its absence and the “1” subscript indicates its presence) taken together with R₁ forms a nitrogen-containing heterocycle, Y is selected from oxygen (O) and sulfur (S), and Z is a leaving group.

With respect to the water-soluble polymer portion, the water-soluble polymer can comprise any polymer so long as the polymer is water-soluble and non-peptidic. Although preferably a poly(ethylene glycol), the water-soluble polymer can be, for example, other water-soluble polymers such as other poly(alkylene oxides), such as copolymers of ethylene glycol and propylene glycol and the like, poly(vinyl pyrrolidone), poly(vinyl alcohol), polyoxazoline, poly(acryloylmorpholine), polyacrylic acid, polyacrylamides, N-(2-hydroxypropyl) methylacrylamide, divyinyl ether-maleic anhydride, polyphosphates, polyphosphazenes, and co-polymers and ter-polymers thereof. The water-soluble polymer can be a homopolymer, or, as mentioned above, can be a copolymer or a terpolymer; such copolymers or terpolymers can be non-random or random. In addition, a water-soluble polymer may be linear, but can also be in other geometric forms such as branched, forked, multi-armed, and the like. Further, poly(alkylene oxide) polymers such as poly(ethylene glycol) are typically terminally capped with an end-capping moiety selected from the group consisting of alkoxy, substituted alkoxy, alkenoxy, substituted alkenoxy, alkynoxy, substituted alkynoxy, aryloxy and substituted aryloxy. Preferred end capping groups include lower alkoxy (C1-C6 alkoxy) and benzyloxy; a particularly preferred terminal capping group is methoxy, and such terminally capped poly(ethylene glycol) polymers are often referred to as methoxy poly(ethylene glycols) or mPEGs. Alternatively, POLY may comprise a functional group or reactive moiety at a terminus thereof, including, but not limited to, for example, hydroxy, amino, ester, carbonate, aldehyde, alkenyl, acrylate, methacrylate, acrylamide, sulfone, thiol, carboxylic acid, isocyanate, isothiocyanate, hydrazide, maleimide, vinylsulfone, dithiopyridine, vinylpyridine, iodoacetamide, silanes, lipids, phospholipids, biotin, fluorescein, and the like, wherein a reactive group may be in a protected form, for example, to allow for further coupling or transformation at a terminus distal to leaving group, “Z”.

As described above, the water-soluble polymer portion of the reagent may possess any of a number of geometric forms, such as linear, branched, multi-armed, etc. In some preferred embodiments, POLY is linear. In yet some other embodiments, POLY is branched. A branched polymer, for example, may comprise a central core radical or branch point from which extends two water-soluble polymeric “arms” or chains. For instance, a branched or multi-armed

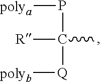

polymer, “POLY” may have a generalized structure such as follows: where polyₐ and poly_{b} are water-soluble polymers as described herein (and may be the same or different), such as for example, methoxy poly(ethylene glycol); R″ is a non-reactive moiety, such as H, methyl or an additional water-soluble polymer, poly_{c}. (e.g., methoxy poly(ethylene glycol), where poly_{c} may be the same or different from polyₐ and/or poly_{b}), and P and Q are nonreactive linkages.

One illustrative branched water-soluble polymer (POLY) is a lysine-branched PEG having a structure as follows, where two poly(ethylene glycol) chains are attached via carbamate linkages to the amino groups of lysine, and the CH carbon provides a site for further modification:

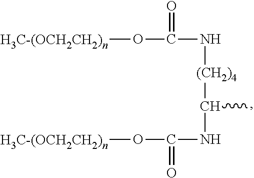

where the value of n may fall within any one or more of the illustrative molecular weight ranges provided herein.

Another illustrative POLY having a branched structure is the following:

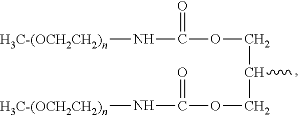

where the central core radical from which the poly(ethylene glycol) chains emanate is (˜HC(CH₂O—)₂), and both of the poly(ethylene glycol) chains are linked via carbamate linkages to the core radical, wherein the carbamate nitrogen atoms are proximal to the polymer chains, and the value of n may fall within any one or more of the illustrative molecular weight ranges provided herein. Alternatively, a branched POLY may have a configuration such as the foregoing structure wherein the orientation of the carbamate linkages is reversed, and the central core radical is (˜HC(CH₂NH—)₂),

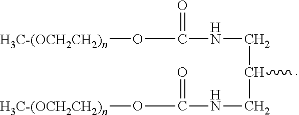

Any of a number of branched poly(ethylene glycol) moieties may be similarly envisioned, and the disclosure is not limited in this regard.

Additional illustrative branched water-soluble polymers may possess any of a number of molecular arrangements, such as for example,

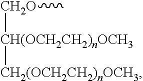

where the value of n may fall within any one or more of the illustrative molecular weight ranges provided herein. In each of the foregoing branched polymer structures, “ [symbol] ” indicates an attachment to (X)_{0,1} as in Formula (I). Various exemplary linking moieties (X) are described in detail herein. Any of a number of organic molecules may be used as a core from which two or more polymer chains emanate to provide a branched or multi-armed polymer, POLY. Suitable core radicals include, but are not limited to, polyols, polythiols, and polyamines. Illustrative polyol core radicals include those derived from glycerol, trimethylolpropane, reducing sugars such as sorbitol or pentaerythritol, and glycerol oligomers, such as hexaglycerol; polythiol and polyamino core radical counterparts of the foregoing polyols may similarly be used.

Although the weight average molecular weight of the water-soluble polymer can vary, the weight average molecular weight of any given water-soluble polymer will typically be in a range of from about 100 daltons to about 200,000 daltons, or from about 100 daltons to about 150,000 daltons, or from about 100 daltons to about 100,000 daltons. In some further embodiments, exemplary weight average molecular weight ranges for POLY, are, for example, from about 120 daltons to about 100,000 daltons (e.g., where (n) for a poly(ethylene glycol) ranges from about 3 to about 2272), or from about 250 daltons to about 60,000 daltons (e.g., where (n) for a poly(ethylene glycol) ranges from about 4.5 to about 1363), or from about 120 daltons to about 6,000 daltons (e.g., where (n) for a poly(ethylene glycol) ranges from about 3 to about 136), or from about 6,000 daltons to about 80,000 daltons (e.g., where (n) for a poly(ethylene glycol) ranges from about 136 to about 1818), or from about 5,000 to about 25,000 daltons (e.g., where (n) for a poly(ethylene glycol) ranges from about 113 to about 568), or from about 10,000 to about 25,000 daltons (e.g., where (n) for a poly(ethylene glycol) ranges from about 227 to about 568). As would be appreciated by one of skill in the art, the illustrative values for “(n)” are calculated for a linear poly(ethylene glycol) chain. Thus, for example, for embodiments in which POLY is a branched poly(ethylene glycol) comprising two polymer chains, where the polymer chains are the same, and the branched poly(ethylene glycol) has an overall weight average molecular weight of about 20,000 daltons, it would be understood that each of the two polymer chains comprising the branched polymer will possess a weight average molecular weight of about 10,000 daltons, such that the value of “(n)” in each polymer chain would be about 227. Similar calculations can be carried out for multi-armed polymers comprising three or more polymer chains.

In yet some other embodiments, POLY, for example, has a weight average molecular weight in a range of from about 200 daltons to about 80,000 daltons, or from about 500 daltons to about 70,000 daltons, or from about 1,000 daltons to about 60,000 daltons, or from about 5,000 daltons to about 25,000 daltons, or from about 5,000 daltons to about 30,000 daltons, or from about 5,000 daltons to about 50,000 daltons, or from about 10,000 daltons to about 60,000 daltons, or from about 10,000 daltons to about 50,000 daltons, or from about 20,000 daltons to about 50,000 daltons, or from about 20,000 daltons to about 40,000 daltons, or from about 20,000 daltons to about 80,000 daltons.

Exemplary ranges, however, include weight-average molecular weights in the following ranges: from about 880 daltons to about 5,000 daltons (e.g., where (n) for a poly(ethylene glycol) ranges from about 20 to about 113); in a range of greater than 5,000 daltons to about 100,000 daltons (e.g., where (n) for a poly(ethylene glycol) ranges from about 113 to about 2272); in a range of from about 6,000 daltons to about 90,000 daltons (e.g., where (n) for a poly(ethylene glycol) ranges from about 136 to about 2045); in a range of from about 10,000 daltons to about 85,000 daltons (e.g., where (n) for a poly(ethylene glycol) ranges from about 227 to about 1932); in a range of greater than 10,000 daltons to about 85,000 daltons (e.g., where (n) for a poly(ethylene glycol) ranges from about 230 to about 1932); in a range of from about 20,000 daltons to about 85,000 daltons (e.g., where (n) for a poly(ethylene glycol) ranges from about 454 to about 1932); in a range of from about 25,000 daltons to about 120,000 daltons (e.g., where (n) for a poly(ethylene glycol) ranges from about 568 to about 2727); in a range of from about 29,000 daltons to about 120,000 daltons (e.g., where (n) for a poly(ethylene glycol) ranges from about 659 to about 2727); in a range of from about 35,000 daltons to about 120,000 daltons (e.g., where (n) for a poly(ethylene glycol) ranges from about 795 to about 2727); in a range of about 880 daltons to about 60,000 daltons (e.g., where (n) ranges for a poly(ethylene glycol) from about 20 to about 1364); in a range of about 440 daltons to about 40,000 daltons (e.g., where (n) for a poly(ethylene glycol) ranges from about 10 to about 909); in a range of about 440 daltons to about 30,000 daltons (e.g., where (n) for a poly(ethylene glycol) ranges from about 10 to about 682); in a range of about 10,000 daltons to about 25,000 daltons (e.g., where (n) for a poly(ethylene glycol) ranges from about 227 to about 568), or in a range of about 15,000 daltons to about 25,000 daltons (e.g., where (n) for a poly(ethylene glycol) ranges from about 341 to about 568), and in a range of from about 40,000 daltons to about 120,000 daltons (e.g., where (n) for a poly(ethylene glycol) ranges from about 909 to about 2727).

Exemplary weight-average molecular weights for POLY include, for example, about 100 daltons, about 120 daltons, about 200 daltons, about 250 daltons, about 300 daltons, about 400 daltons, about 440 daltons, about 500 daltons, about 600 daltons, about 700 daltons, about 750 daltons, about 800 daltons, about 900 daltons, about 1,000 Daltons, about 1,500 daltons, about 2,000 daltons, about 2,200 daltons, about 2,500 daltons, about 3,000 daltons, about 4,000 daltons, about 4,400 daltons, about 4,500 daltons, about 5,000 daltons, about 5,500 daltons, about 6,000 daltons, about 7,000 daltons, about 7,500 daltons, about 8,000 daltons, about 9,000 daltons, about 10,000 daltons, about 11,000 daltons, about 12,000 daltons, about 13,000 daltons, about 14,000 daltons, about 15,000 daltons, about 16,000 daltons, about 17,000 daltons, about 18,000 daltons, about 19,000 daltons, about 20,000 daltons, about 22,500 daltons, about 25,000 daltons, about 30,000 daltons, about 35,000 daltons, about 40,000 daltons, about 45,000 daltons, about 50,000 daltons, about 55,000 daltons, about 60,000 daltons, about 65,000 daltons, about 70,000 daltons, and about 75,000 daltons, about 80,000 daltons, about 85,000 daltons, about 90,000 daltons, about 95,000 daltons, and about 100,000 daltons.

In yet some particular embodiments, POLY has a weight average molecular weight selected from 200 daltons, 300 daltons, 400 daltons, 500 daltons, 750 daltons, 1,000 daltons, 2,500 daltons, 3,000 daltons, 5,000 daltons, 7500 daltons, 10,000 daltons, 15,000 daltons, 20,000 daltons, 25,000 daltons, 30,000 daltons, 40,000 daltons, 50,000 daltons, 55,000 daltons, 60,000 daltons, and 65,000 daltons.

As described above, POLY is preferably polyethylene glycol. When a PEG is used as the water-soluble polymer in the polymeric reagent, the PEG typically comprises a number of (OCH₂CH₂) monomers (or (CH₂CH₂O) monomers, depending on how the PEG is defined). As used throughout the description, the number of repeating units is identified by the subscript “n” in “(OCH₂CH₂)ₙ”. Thus, the value of (n) typically falls within one or more of the ranges provided herein. For any given polymer in which the molecular weight is known, it is possible to determine the number of monomeric repeating units (i.e., “n”) by dividing the total weight-average molecular weight of the polymer by the molecular weight of the repeating monomer. For example, when POLY is a polyethylene glycol having a molecular weight of about 10,000 Daltons, the value of (n) is about 227; when POLY is a polyethylene glycol having a molecular weight of about 20,000 daltons, the value of (n) is about 454, and so forth.

In reference to Formula (I), the polymer reagent may comprise a linker moiety, X. The linker moiety, when present, is interposed between the water-soluble polymer and either the nitrogen-containing heterocycle formed between R₂ and R₁N

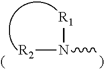

or NR₁, depending upon the presence or absence of R₂. In some embodiments, X is absent. In one or more further embodiments, both X and R₂ are absent. In some other embodiments, X is present. In one or more further embodiments, both X and R₂ are present. Preferably, X is a hydrolytically stable linker. The linker moiety may comprise a single atom or a collection of atoms. Generally speaking, the linker moiety (sometimes referred to herein simply as the “linker”), X, has an atom length of from about one atom to about twenty-five atoms, or from about one atom to about twenty atoms. In some embodiments, the linker moiety has a chain length of about two atoms to about ten atoms. Representative linkers have a length of about one, two, three, four, five, six, seven, eight, nine or ten atoms. In referring to a chain length of a collection of atoms, —CH₂—, for example, is considered as having a length of one atom, although the methylene group itself contains three atoms total, since the hydrogen atoms are substituents on the carbon, and —CH₂CH₂—, for instance, is considered as having a chain length of two carbon atoms, etc. Similarly, —OC(O)— is considered to have a length of two atoms, while —OC(O)—NH— is considered to have a length of three atoms, and so forth. Illustrative linker moieties can include atoms or groups of atoms such as those selected from, for example, —O—, —S—, —NH, —C(O)—, —O—C(O)—, —C(O)—O—, —C(O)—NH—, —NH—C(O)—NH—, —O—C(O)—NH—, —O—C(O)-NHCH₂—, —C(S)—, —CH₂—, —CH₂—CH₂—, —CH₂—CH₂—CH₂—, —CH₂—CH₂—CH₂—CH₂, —O—CH₂—, —CH₂—O—, —O—CH₂—CH₂—, —CH₂—O—CH₂—, —CH₂—CH₂—O—, —O—CH₂—CH₂—CH₂—, —CH₂—O—CH₂—CH₂—, —CH₂—CH₂—O—CH₂—, —CH₂—CH₂—CH₂—O—, —O—CH₂—CH₂—CH₂—CH₂—, —CH₂—O—CH₂—CH₂—CH₂—, —CH₂—CH₂—O—CH₂—CH₂—, —CH₂—CH₂—CH₂—O—CH₂—, —CH₂—CH₂—CH₂—CH₂—O—, —C(O)—NH—CH₂—, —C(O)—NH—CH₂—CH₂—, —CH₂—C(O)—NH—CH₂—, —CH₂—CH₂—C(O)—NH—, —C(O)—NH—CH₂—CH₂—CH₂—, —CH₂—C(O)—NH—CH₂—CH₂—, —CH₂—CH₂—C(O)—NH—CH₂—, —CH₂—CH₂—CH₂—C(O)—NH—, —C(O)—NH—CH₂—CH₂—CH₂—CH₂—, —CH₂—C(O)—NH—CH₂—CH₂—CH₂—, —CH₂—CH₂—C(O)—NH—CH₂—CH₂—, —CH₂—CH₂—CH₂—C(O)—NH—CH₂—, —CH₂—CH₂—CH₂—C(O)—NH—CH₂—CH₂—, —CH₂—CH₂—CH₂—CH₂—C(O)—NH—, —C(O)—O—CH₂—, —CH₂—C(O)—O—CH₂—, —CH₂—CH₂C(O)—O—CH₂—, —C(O)—O—CH₂—CH₂—, —NH—C(O)—CH₂—, —CH₂—NH—C(O)—CH₂—, —CH₂—CH₂—NH—C(O)—CH₂—, —NH—C(O)—CH₂—CH₂—, —CH₂—NH—C(O)—CH₂—CH₂—, —CH₂—CH₂—NH—C(O)—CH₂—CH₂—, —C(O)—NH—CH₂—, —C(O)—NH—CH₂—CH₂—, —O—C(O)—NH—CH₂—, —O—C(O)—NH—CH₂CH₂, —O—C(O)—NH—CH₂—CH₂—CH₂—, —NH—CH₂—, —NH—CH₂—CH₂—, —CH₂—NH—CH₂—, —CH₂—CH₂—NH—CH₂—, C(O)CH₂—, —C(O)—CH₂—CH₂—, —CH₂—C(O)—CH₂—, —CH₂—CH₂C(O)—CH₂—, —CH₂—CH₂—C(O)—CH₂—CH₂—, —CH₂—CH₂—C(O)—, —CH₂—CH₂—CH₂—C(O)—NH—CH₂—CH₂—NH—, —CH₂—CH₂—CH₂—C(O)—NH—CH₂—CH₂—NH—C(O)—, —CH₂—CH₂—CH₂—C(O)—NH—CH₂—CH₂—NH—C(O)—CH₂—, —CH₂—CH₂—CH₂—C(O)—NH—CH₂—CH₂—NH—C(O)—CH₂—CH₂—, —O—C(O)—NH—(CH₂)₀-6—(OCH₂CH₂)₀₋₂—, C(O)—NH—(CH₂)₁₋₆—NH—C(O)—, —NH—C(O)—NH—(CH₂)₁₋₆—NH—C(O)—, —O—C(O)—CH₂—, —O—C(O)—CH₂—CH₂—, —O—C(O)—CH₂—CH₂—CH₂—, —S—C(S)—, —C(S)S—, —C(S)—NH—, —NH—C(S)—NH—, —S—C(S)—NH—, —S—C(S)—NHCH₂, —C(S)—, —S—CH₂—, —CH₂—S—, —S—CH₂—CH₂—, —CH₂—S—CH₂—, —CH₂—CH₂—S—, —S—CH₂—CH₂—CH₂—, —CH₂—S—CH₂—CH₂—, —CH₂—CH₂—S—CH₂—, —CH₂—CH₂—CH₂—S—, —S—CH₂—CH₂—CH₂—CH₂—, —CH₂—S—CH₂—CH₂—CH₂—, —CH₂—CH₂—S—CH₂—CH₂—, —CH₂—CH₂—CH₂—S—CH₂—, —CH₂—CH₂—CH₂—CH₂—S—, —C(S)—NH—CH₂—, —C(S)—NH—CH₂—CH₂—, —CH₂—C(S)—NH—CH₂—, —CH₂—CH₂—C(S)—NH—, —C(S)—NH—CH₂—CH₂—CH₂—, —CH₂—C(S)—NH—CH₂—CH₂—, —CH₂—CH₂—C(S)—NH—CH₂—, —CH₂—CH₂—CH₂—C(S)—NH—, —C(S)—NH—CH₂—CH₂—CH₂—CH₂—, —CH₂—C(S)—NH—CH₂—CH₂—CH₂—, —CH₂—CH₂—C(S)—NH—CH₂—CH₂—, —CH₂—CH₂—CH₂—C(S)—NH—CH₂—, —CH₂—CH₂—CH₂—C(S)—NH—CH₂—CH₂—, —CH₂—CH₂—CH₂—CH₂—C(S)—NH—, —C(S)—S—CH₂—, —CH₂—C(S)—S—CH₂—, —CH₂—CH₂C(S)—S—CH₂—, —C(S)—S—CH₂—CH₂—, —NH—C(S)—CH₂—, —CH₂—NH—C(S)—CH₂—, —CH₂—CH₂—NH—C(S)—CH₂—, —NH—C(S)—CH₂—CH₂—, —CH₂—NH—C(S)—CH₂—CH₂—, —CH₂—CH₂—NH—C(S)—CH₂—CH₂—, —C(S)—NH—CH₂—, —C(S)—NH—CH₂—CH₂—, —S—C(S)—NH—CH₂—, —S—C(S)—NH—CH₂CH₂, —S—C(S)—NH—CH₂—CH₂—CH₂—, —C(S)CH₂—, —C(S)—CH₂—CH₂—, —CH₂—C(S)—CH₂—, —CH₂—CH₂C(S)—CH₂—, —CH₂—CH₂—C(S)—CH₂—CH₂—, —CH₂—CH₂—C(S)—, —CH₂—CH₂—CH₂—C(S)—NH—CH₂—CH₂—NH—, —CH₂—CH₂—CH₂—C(S)—NH—CH₂—CH₂—NH—C(S)—, —CH₂—CH₂—CH₂—C(S)—NH—CH₂—CH₂—NH—C(S)—CH₂—, —CH₂—CH₂—CH₂—C(S)—NH—CH₂—CH₂—NH—C(S)—CH₂—CH₂—, —S—C(S)—NH—(CH₂)₀₋₆—(OCH₂CH₂)₀₋₂—, C(S)—NH—(CH₂)₁₋₆—NH—C(S)—, —NH—C(S)—NH—(CH₂)₁₋₆—NH—C(S)—, —S—C(S)—CH₂—, —S—C(S)—CH₂—CH₂—, —S—C(S)—CH₂—CH₂—CH₂—. A linker moiety may also comprise a combination of any two or more of the foregoing atoms or groups of atoms, in any orientation.

In some embodiments, the linker moiety is characterized by a general structure: ˜(CH₂)ₐ(O)[C(O)](NH)ₐ(CH₂)˜, where (a) is an integer having a value of from 0-6; (b) is 0 or 1; (c) is 0 or 1; (d) is 0 or 1; and (e) is an integer having a value from 0-6, wherein at least one of (a), (b), (c), (d), and (e) is a positive integer (i.e., is not zero). For example, in some embodiments, (a) is zero, and (b), (c), (d), and (e) are all non-zero, so that the linker possesses the structure: ˜ OC(O)NH(CH₂)₁₋₆˜, with illustrative linkers having a structure selected from: ˜OC(O)NHCH₂˜˜ OC(O)NH(CH₂)₂˜, ˜OC(O)NH(CH₂)₃˜, ˜OC(O)NH(CH₂)+˜, ˜OC(O)NH(CH₂)₅˜, and ˜OC(O)NH(CH₂)˜. In some other embodiments, both (a) and (e) are zero, so that the linker has a structure: ˜ OC(O)NH ˜. In some other embodiments, (a), (d), and (e) are zero, while (b) and (c) are both one, such that the linker has a structure: ˜ OC(O) ˜. In some other embodiments, (a), (b), (c) and (d) are one, and (e) is zero, such that the linker has a structure: ˜ (CH₂)₁₋₆OC(O)NH˜, and so forth. Thus, in some embodiments, the linker moiety is selected from ˜OC(O)˜, ˜OC(O)NH˜, and ˜O—C(O)—NH—(CH₂)₁₋₆˜(i.e., ˜O—C(O)—NH—(CH₂)˜.˜O—C(O)—NH—(CH₂)₂˜˜ ˜ O—C(O)—NH—(CH₂)₃˜˜ ˜ O—C(O)—NH—(CH₂)+˜˜ ˜ O—C(O)—NH—(CH₂)˜, or ˜O—C(O)—NH—(CH₂)₆˜). See, for example, exemplary Reagents 2′, 7′, 8′ and 9′.

In reference to the polymer reagent of Formula (1),

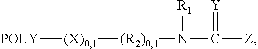

R₁ is an organic radical attached to a nitrogen atom (i.e., the nitrogen atom that is adjacent to ˜C(Y)Z), and may, in some cases, form a nitrogen-containing heterocycle when taken together with R₂

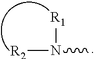

R₂ is either absent, or when present, taken together with R₁ (that is, R₁—N), forms a nitrogen-containing heterocycle. R₁ may be selected from, for example, substituted and unsubstituted alkyl, substituted and unsubstituted cycloalkyl, substituted and unsubstituted alkenyl, substituted and unsubstituted cycloalkenyl, substituted and unsubstituted alkynyl, substituted and unsubstituted heteroalkyl, substituted and unsubstituted cycloheteroalkyl, substituted and unsubstituted aryl, substituted and unsubstituted aralkyl, substituted and unsubstituted heteroaryl, and substituted and unsubstituted heteroaralkyl. Illustrative organic radicals include alkyl, cycloalkyl, alkenyl, cycloalkenyl, alkynyl, heteroalkyl, cycloheteroalkyl, aryl, aralkyl, heteroaryl, and heteroaralkyl, each optionally substituted with one or more substituents independently selected from halo, alkylhalo, hydroxy, alkylhydroxy, alkoxy, amino, alkylamino, sulfhydryl, alkylsulfhydryl, nitro, alkylnitro, cyano, alkylcyano, thiocyano, alkylthiocyano, imino, alkylimino, carbamate, alkylcarbamate, phosphate, alkylphosphate, alkylcarbonyl, carboxamide, alkylcarboxamide, alkoxycarbonyl, thioalkyl, thioester, and alkylthioester.

In some particular embodiments, e.g., when R₂ is absent, R₁ is selected from lower alkyl (C1-C6 alkyl), halo-substituted (e.g., fluoro, chloro, bromo, or iodo-substituted) lower alkyl, benzyl, halo-substituted benzyl, and nitro-substituted benzyl. In preferred embodiments, a halo-substituted group, such as for example, a halo-substituted lower alkyl or a halo-substituted benzyl, is substituted with one or more fluoro or chloro atoms. For example, a halo-substituted benzyl may have one, two, three, four or five halo substituents on the benzyl ring, and/or may possess one or more halo substituents on the benzyl methylene group. Substituents on the benzyl phenyl ring may be in any location, that is, on any of the available ring carbons. In some embodiments, one or more substituents on R₁ are electron withdrawing. In some further embodiments, one or more substituents on R₁ are fluorine. Exemplary substituted R₁ groups include for example, ˜CH₂X, CHX₂, ˜CH₂CH₂X, ˜CH₂CHX₂, ˜CH₂CX₃. CHXCH₃, ˜CX₂CH₃, ˜CHXCH₂X, ˜CHXCHX₂, CHXCX₃. ˜ CH₂CH₂CH₂X, ˜CH₂CH₂CHX₂, ˜CH₂CH₂CX₃, where each X is independently halo (e.g., F, Cl, Br, I). In some embodiments, each X is the same halo. In some preferred embodiments, each X is fluoro. See, for example, illustrative Reagents 3′, 4′, 5′, and 6′, with R₁ groups ˜CH₂CH₂F, ˜CH₂CHF₂, ˜CH₂CHF₃, and ˜CH₂C6F₅, respectively.

In some other embodiments, R₂ is present, and when taken together with R₁—N, forms a nitrogen-containing heterocycle, i.e., a non-aromatic nitrogen-containing heterocycle, wherein the nitrogen-containing heterocycle may be substituted or unsubstituted. The nitrogen-containing heterocycle may be saturated or unsaturated, and one of the heterocyclic ring atoms (other than N-R₁) is attached to X (if present) or to POLY. In one or more preferred embodiments, the nitrogen-containing heterocycle is saturated. Suitable nitrogen-containing heterocycles may contain, for example, 4, 5, 6, or 7 heterocycle ring atoms, and may contain up to three nitrogen (e.g., one, two or three) nitrogen ring atoms. In some embodiments, the nitrogen-containing heterocycle contains one nitrogen atom (i.e., ˜N-R₁). In yet some other embodiments, the nitrogen-containing heterocycle contains two nitrogen atoms. The nitrogen-containing heterocycle may be optionally substituted with one or more substituents selected from the group consisting of alkyl, substituted alkyl, alkenyl, substituted alkenyl, alkynyl, substituted alkynyl, heteroalkyl, substituted heteroalkyl, aralkyl, substituted aralkyl, halo, alkylhalo, hydroxy, alkylhydroxy, alkoxy, amino, alkylamino, sulfhydryl, alkylsulfhydryl, nitro, alkylnitro, cyano, alkylcyano, thiocyano, alkylthiocyano, imino, alkylimino, carbamate, alkylcarbamate, phosphate, alkylphosphate, alkylcarbonyl, carboxamide, alkylcarboxamide, alkoxycarbonyl, thioalkyl, thioester, and alkylthioester. In some embodiments, the nitrogen-containing heterocycle is substituted at one or more ring positions with lower alkyl, substituted lower alkyl, aralkyl, or substituted aralkyl, such as, for example, halo-substituted lower alkyl or halo-substituted aralkyl. In some preferred embodiments, a substituted nitrogen-containing heterocycle is either mono-substituted or is di-substituted. In some particular embodiments, a substituted nitrogen-containing heterocycle is mono-substituted. In some even more particular embodiments, the nitrogen-containing heterocycle is mono-substituted with fluoro-substituted lower alkyl. In one or more additional preferred embodiments, when R₂ is present (and thus when taken together with R₁—N, forms a nitrogen-containing heterocycle), X is also present.

Exemplary nitrogen-containing heterocycles formed by taking R₂ together with R₁ include azetidine, substituted azetidine, diazetidine, substituted diazetidine, pyrrolidine, substituted pyrrolidine, imidazolidine, substituted imidazolidine, piperidine, substituted piperidine, morpholine, substituted morpholine, diazinanes (1,2-, 1,3-, 1,4-diazinane) substituted diazinanes, triazinanes (1,2,3-, 1,2,4-, 1,3,5-), substituted triazinanes, azepane, substituted azepane, diazepanes (1,2-, 1,3-, 1,4-), and substituted diazepanes, and the like, as shown below for the exemplary unsubstituted nitrogen containing heterocycles, where www indicates attachment to —C(Y)Z and any one or more ring positions may contain a substituent as described above. It will be understood that for the illustrative nitrogen-containing heterocycles shown below, at a carbon or nitrogen ring atom attached to POLY-(X)_{0.1}˜, a hydrogen will be displaced by a bond to POLY-(X)_{0.1}˜.

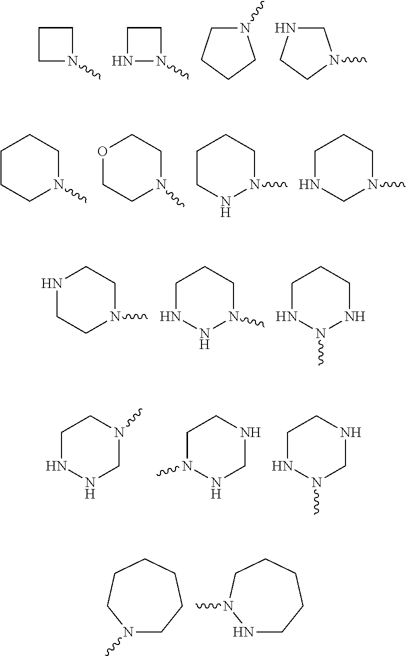

In some preferred embodiments, R₂ together with R₁ forms a piperidine or a substituted piperidine. In some other preferred embodiments, R₂ together with R₁ forms a diazinane or a substituted diazinane, such as, for example, piperazine or substituted piperazine.

Thus, exemplary polymer reagents such as provided in the accompanying examples comprise the following nitrogen-containing heterocycles,

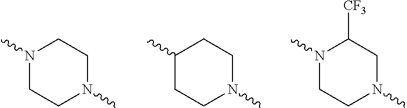

in accordance with Formula (I), to form the corresponding polymer reagents:

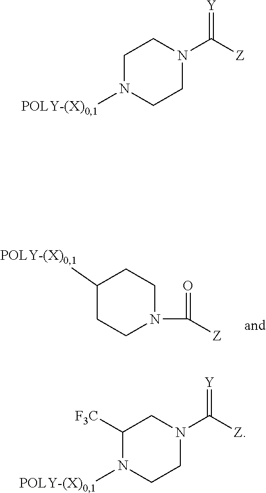

See, for example, Reagents 2, 7, 8 and 9.

In reference to formula (I), Y is oxygen (O) or sulfur (S). In some preferred embodiments, Y is O.

As described above, the polymer reagent includes a leaving group, Z. A leaving group is an atom or collection of atoms (e.g., a functional group) that is displaced from the remainder of the molecule during a reaction such as a substitution reaction. A leaving group, Z, may be comprised within a reactive group of the polymeric reagent formed by ˜C(Y) taken together with Z. For example, upon reaction of the polymer reagent (e.g., in a bioconjugation reaction) with an active agent bearing an amino group such as a histidine amino group, Z acts as a leaving group. Suitable reactive groups include, for example, tetrazoles, isocyanates, isothiocyanates, N-hydroxysuccinimidyl esters, carbodiimide, acyl azide, carbonates, imidoesters, fluorophenyl ester, benzotriazoles, and para-nitrophenyl carbonate, among others. Examples of such reactive groups include N-hydroxysuccinimidyl (NHS) ester, NHS carbonate ester, succinimidyl succinate, succinimidyl glutarate, para-nitrophenyl carbonate, and benzotriazole carbonate. Suitable leaving groups, Z, include, for example, tetrazoles, isocyanates, isothiocyanates, N-hydroxysuccinimide, acyl azide, fluorophenol, benzotriazoles, nitrophenols, and triazoles, among others.

In one or more embodiments, preferred leaving groups include, for example, tetrazoles such as a phenyl tetrazole and benzotriazoles. In some particularly preferred embodiments, the leaving group is a substituted benzotriazole or phenyl tetrazole or a substituted phenyl tetrazole, such as 5-[3,5-bis(trifluoromethyl)phenyl]-2H-tetrazole (BTMP).

A phenyl tetrazole leaving group may have a structure such as follows:

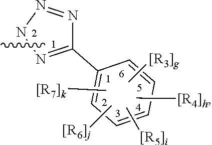

wherein g, h, i, j, and k is each independently 0 or 1 (wherein 0 indicates absence and 1 indicates presence), and R₃, R₄, R₅, R₆ and R₇ is each independently selected from the group consisting of alkyl, substituted alkyl, alkenyl, substituted alkenyl, alkynyl, substituted alkynyl, heteroalkyl, substituted heteroalkyl, aralkyl, substituted aralkyl, halo, alkylhalo, hydroxy, alkylhydroxy, alkoxy, amino, alkylamino, sulfhydryl, alkylsulfhydryl, nitro, alkylnitro, cyano, alkylcyano, thiocyano, alkylthiocyano, imino, alkylimino, carbamate, alkylcarbamate, phosphate, alkylphosphate, alkylcarbonyl, carboxamide, alkylcarboxamide, alkoxycarbonyl, thioalkyl, thioester, and alkylthioester. For example, the phenyl tetrazole leaving group may be unsubstituted (i.e., , h, i, j, and k are each 0). Alternatively, the phenyl tetrazole may have a single substituent on the phenyl ring (e.g., at carbon 2 (C2), at carbon 3 (C3), or at carbon 4 (C4)). In some further embodiments, the phenyl ring had two substituents (e.g., at carbon 2 (C2) and carbon 3 (C3), at carbon 2 (C2) and carbon 4 (C4), at carbon 3 (C3) and carbon 5 (C5), at carbon 3 (C3) and carbon 4 (C4), or at carbon 2(C2) and carbon 6 (C6)). The phenyl tetrazole may also possess three substituents on the phenyl ring (e.g., at C2, C3 and C4; or at C2, C3, and C5; or at C2, C3 and C6; or at C2, C4, and C6; or at C3, C4 and C5; or at C3, C4, and C6). In yet some further embodiments, the phenyl tetrazole leaving group may possess four substituents on the phenyl ring (e.g., at C2, C3, C4 and C5; or at C2, C3, C5 and C6; or at C2, C3, C4 and C6). In yet some additional embodiments, the phenyl tetrazole leaving group may possess five substituents on the phenyl ring. Regarding substituted phenyl tetrazoles as described above having more than one substituent on the phenyl ring, the substituents may be the same or may differ (or may be a combination, where two or more substituents are the same, and further substituents differ from the former). In some preferred embodiments, one or more substituents on the phenyl ring are trifluoromethyl. In more particular preferred embodiments, the leaving group is a di-substituted phenyl tetrazole with trifluoromethyl substituents at C3 and C5 (i.e., is 5-[3,5-bis(trifluoromethyl)phenyl]-2H-tetrazole (BTMP)) as shown below:

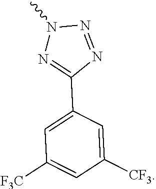

Exemplary histidine-selective polymeric reagents are further described in Table 1 and in the examples. As provided in Table 1, polymer reagents having a variety of structural features have been prepared. Illustrative polymer reagents in accordance with the instant disclosure are shown below, wherein POLY is a water-soluble polymer (preferably a poly(ethylene glycol) polymer) and LG is a leaving group as previously described:

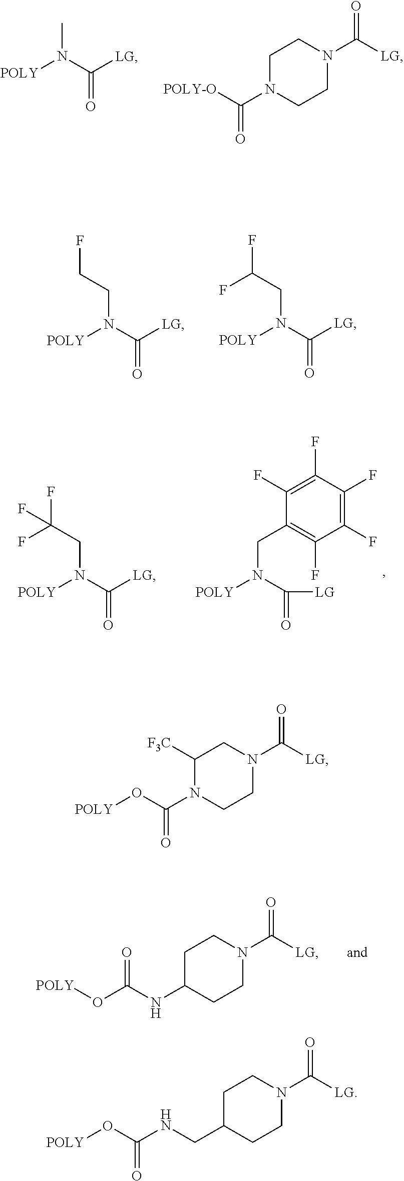

More particular illustrative poly(ethylene glycol) polymer reagents include the following:

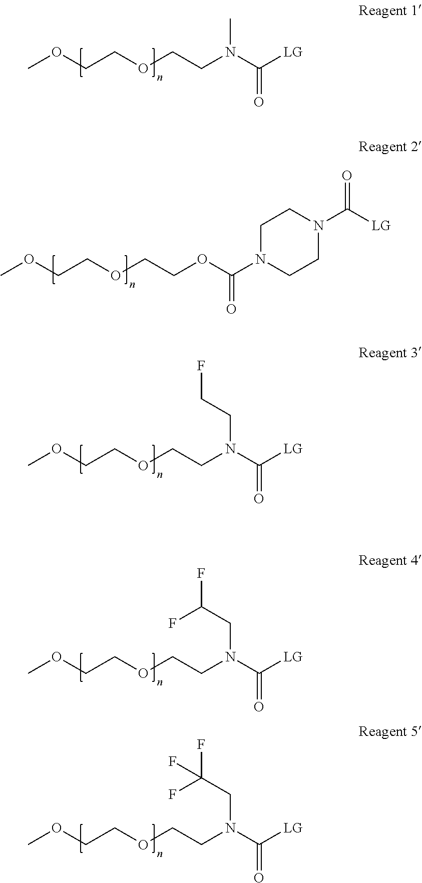

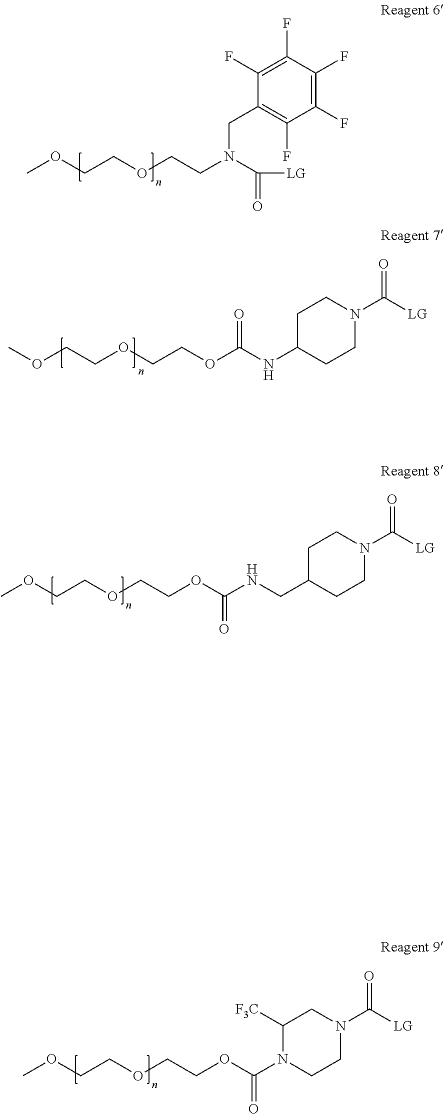

wherein each (n) is independently in a range selected from the group consisting of: from about 2 to about 2,273; from about 4 to about 1800; from about 11-1590; from about 23 to about 1363; from about 113 to about 568; from about 113 to about 682; from about 113 to about 1136; from about 227 to about 1363; from about 227 to about 1136; from about 454 to about 1136; from about 454 to about 909; and from about 454 to about 1818; and LG is a leaving group as previously described. In one or more preferred embodiments, the leaving group is 5-[3,5-bis(trifluoromethyl)phenyl]-2H-tetrazole (BTMP)).

### Method of Making

Histidine-selective polymeric reagents having the general structural and functional features described herein can be synthesized using conventional organic and polymer chemistry techniques in light of the instant disclosure. The syntheses of exemplary polymer Reagents 1-8 (and their respective conjugates) are described in the accompanying examples; additional polymeric reagents according to Formula I may be similarly prepared. It will be understood that synthetic strategies and approaches different from those exemplified herein may also be employed to prepare polymeric reagents in accordance with the invention, and that based upon the structural and supporting information provided herein, such alternative syntheses are well within the level of one skilled in the art of synthetic organic chemistry.

Histidine-selective polymer reagents in accordance with the instant disclosure can be prepared from a suitable water-soluble polymeric starting material. Taking the water-soluble polymer, polyethylene glycol, as a preferred example (with the recognition that any suitable water-soluble polymer can be similarly functionalized), the subject histidine-selective polymer reagents can be prepared from a starting material such as poly(ethylene glycol), or preferably, from an end-capped poly(ethylene glycol) such as, e.g., methoxy(polyethylene glycol), wherein the polymer may be linear, branched, etc., as described previously herein, to thereby introduce to the polymer the features of ˜(X)_{0.1}—(R₂)_{0.1}—N(R₁)—C(Y)—Z.

For example, in synthesizing a polymer reagent in accordance with Formula (I), such as for example, exemplary Reagents 1, 3, 4, 5, and 6 (wherein X and R₂ are absent), a suitable poly(ethylene glycol) starting material such as methoxypolyethylene glycol (“mPEG-OH”) may be reacted with a reagent effective to convert the hydroxy-terminus into a good leaving group. Exemplary leaving groups include tosylate (p-toluene sulfonate), triflate (trifluoromethanesulfonate), mesylate (methanesulfonate), chloride, bromide, and the like, and can be introduced by use of the corresponding reagent. For instance, methoxy(polyethylene glycol) may be reacted with, for example, a mesyl salt, tosyl salt, or triflate salt, such as for example, mesyl chloride, tosyl chloride, or triflate chloride, respectively, or other suitable reagent, under conditions effective to replace the terminal hydroxy with a good leaving group. See, for example, Example 1.1. The resulting polymer intermediate, possessing a good leaving group, may then be reacted with a suitable amine (or corresponding amine salt) to thereby displace the leaving group and introduce the corresponding (˜NR₁) portion of the polymeric reagent, wherein R₁ is an organic radical (as has been described in detail elsewhere herein), and may form a nitrogen-containing heterocycle when taken with R₂. Such conversion may occur by virtue of a single transformation or may require more than one transformation step. In some preferred embodiments, R₁ is lower alkyl, halo-substituted lower alkyl, benzyl, or halo-substituted benzyl, wherein the benzyl ring has from one to five halo-substituents (e.g., fluoro, chloro, or bromo). Exemplary amine reagents include NH₂R₁ (including salt forms thereof). The reaction may be carried out under basic conditions, for example, at pHs ranging from about 11.0 to 14.0, or from about 12 to 14, depending upon the particulars of the chemical transformation(s). The desired polymer-amine intermediate is then typically recovered, and may be further purified, if desired. As an illustration, methylamine hydrochloride was employed to form the polymer amine intermediate of Reagent 1 (mPEG-NHCH₃); 2-fluoroethylamine hydrochloride was used as an amine reactant to form the polymer amine intermediate of Reagent 3 (mPEG-NHCH₂CH₂F); 2,2-difluoroethylamine was used as an amine reactant to form the polymer amine intermediate of Reagent 4 (mPEG-NHCH₂CHF₂), ammonium hydroxide was used as an amine reactant to form a precursor polymer amine intermediate of Reagent 5, mPEG amine, followed by reaction with trifluoroacetic anhydride to thereby facilitate introduction of the trifluoroacetate group onto the amino nitrogen, followed by reduction with a suitable reducing agent (e.g., a borohydride such as sodium cyanoborohydride or a borane such as diborane, or the like) to form the polymer amine intermediate of Reagent 5, mPEG-NHCH₂CF₃. Alternatively, PEG amines having a variety of molecular weights for use as a starting material may also be purchased from vendors such as Creative PEGWorks or NOF America Corporation. A precursor PEG amine, mPEG amine, was also used to prepare the polymer amine intermediate of Reagent 6, mPEG-NHCH₂C6F₅, by reacting mPEG amine with the pentafluorobenzene reactant, pentafluorobenzaldehyde, in the presence of a reducing agent. Other polymer amine intermediates with a variety of R₁ groups may be similarly prepared to prepare polymeric reagents as described herein., wherein suitable R₁ groups include, for example, substituted and unsubstituted alkyl, substituted and unsubstituted cycloalkyl, substituted and unsubstituted alkenyl, substituted and unsubstituted cycloalkenyl, substituted and unsubstituted alkynyl, substituted and unsubstituted heteroalkyl, substituted and unsubstituted cycloheteroalkyl, substituted and unsubstituted aryl, substituted and unsubstituted aralkyl, substituted and unsubstituted heteroaryl, and substituted and unsubstituted heteroaralkyl; more particularly, illustrative R₁ groups include, for example, alkyl, cycloalkyl, alkenyl, cycloalkenyl, alkynyl, heteroalkyl, cycloheteroalkyl, aryl, aralkyl, heteroaryl, and heteroaralkyl, each optionally substituted with one or more substituents independently selected from the group consisting of halo, alkylhalo, hydroxy, alkylhydroxy, alkoxy, amino, alkylamino, sulfhydryl, alkylsulfhydryl, nitro, alkylnitro, cyano, alkylcyano, thiocyano, alkylthiocyano, imino, alkylimino, carbamate, alkylcarbamate, phosphate, alkylphosphate, alkylcarbonyl, carboxamide, alkylcarboxamide, alkoxycarbonyl, thioalkyl, thioester, and alkylthioester.

In synthesizing reagents such as exemplary Reagents 2, 7, 8, and 9 (wherein X and R₂ are both present, and further wherein X comprises an ester function, ˜OC(O)˜, and R₁ and R₂, taken together, form a nitrogen-containing heterocycle), the corresponding histidine-selective reagent may be prepared from a poly(ethylene glycol) activated ester starting material such as, for example, methoxypolyethylene glycol succinimidyl carbonate or methoxypolyethylene glycol chloroformic ester. Such activated water-soluble polymer reagents may then be reacted with an amine-containing compound, such as an amine-comprising heterocycle, to form the corresponding amide, wherein, as in the case of Reagents 2 and 9, the resulting amide amino group forms part of a nitrogen-containing heterocycle (which for exemplary Reagents 2 and 9, is piperazine). Alternatively, the nitrogen atom of the newly-formed amide group may not form part of the nitrogen-containing heterocycle, but may be proximal thereto (see, e.g., Reagent 7) or may possess one or more intervening atoms or series of atoms between it and the nitrogen-containing heterocycle, such as is the case for illustrative Reagent 8. The exemplified reagents comprise X groups such as, for example, —OC(O)—, —OC(O)—NH—, and —OC(O)—NH—(CH₂)₁₋₆, although X is not limited in this regard.

Activated PEG esters can be prepared directly from methoxyPEG-OH (or any other suitably end-capped water-soluble polymer) or can be purchased from a commercial source, such as, for example, Axis Pharm (San Diego, CA) and NOF America Corporation (San Mateo, CA). Preparation of an mPEG activated ester reagent such as mPEG-SC (methoxyPEG succinimidyl carbonate, also referred to as mPEG NHS ester) may be carried out by reacting methoxyPEG-OH with a suitable ester activating agent such as N, N′-disuccinimidyl carbonate in a suitable organic solvent or mixture of solvents. Similarly, mPEG chloroformic ester can be prepared by reacting methoxyPEG-OH with phosgene in a suitable organic solvent or a mixture of solvents under anhydrous conditions. Other activated PEG reagents may be similarly prepared. Following formation of an activated PEG reagent, introduction of a nitrogen containing heterocycle optionally via linker, X, may be carried out, or alternatively, an in-situ ring formation reaction may be conducted. For instance, an mPEG activated ester may be reacted with an amine-containing heterocycle, preferably an amine-containing heterocycle containing four, five, six or seven ring atoms to form, PEG-X—R₂—NR₁H. Illustrative heterocycles include but are not limited to azetidine, substituted azetidine, diazetidine, substituted diazetidine, pyrrolidine, substituted pyrrolidine, imidazolidine, substituted imidazolidine, piperidine, substituted piperidine, diazinanes, substituted diazinanes, triazinanes, substituted triazinanes, azepanes, substituted azepanes, diazepanes and substituted diazepanes; some preferred heterocycles include piperidine and piperazine (including substituted forms thereof). In illustrative Example 2, an mPEG activated ester was reacted with an exemplary nitrogen-containing heterocycle, piperazine, in an organic solvent to form the corresponding nitrogen-containing heterocycle-functionalized PEG intermediate. In Example 7, an mPEG activated ester was reacted with an amino-substituted nitrogen-containing heterocycle, 4-aminopiperidine, in the presence of butylated hydroxytoluene and a base under an inert atmosphere to form the corresponding nitrogen-containing heterocycle-functionalized PEG intermediate, wherein the piperidine amino group was in protected form to promote coupling of the non-ring amino group with the activated PEG reagent. In Example 8, an mPEG activated ester was reacted with an amino-substituted nitrogen-containing heterocycle, more particularly, an alkylamino-substituted nitrogen-containing heterocycle, 4-aminomethyl piperidine, in the presence of butylated hydroxytoluene and a base under an inert atmosphere to form the corresponding nitrogen-containing heterocycle-functionalized PEG intermediate, wherein the piperidine amino group was in protected form to promote coupling of the non-ring amino group with the activated PEG reagent. In Example 9, an activated PEG reagent, methoxypolyethylene glycol chloroformic ester, was reacted with a nitrogen-containing heterocycle comprising a haloalkyl substituent, trifluoromethyl-piperazine in the presence of base in an organic solvent. The reaction was carried out under an inert atmosphere; the 4-amino group of the 2-trifluoromethylpiperazine ring was in protected form. The nitrogen-containing heterocycle reactants, 4-amino-piperidine (e.g., which may be in protected form), 4-aminomethyl-piperidine (e.g., which may be in protected form), and 2-trifluoromethyl-piperazine (e.g., which may be in protected form) are illustrative; other nitrogen-containing heterocycle or substituted nitrogen-containing heterocycle reactants may be similarly employed to form a nitrogen-containing heterocycle-comprising PEG (or other suitable water-soluble polymer) intermediate,

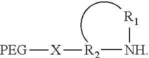

Following removal of a protecting group, if present, the intermediate may then be recovered and optionally further purified. For example, the PEG intermediate may be recovered following precipitation, for example, using a solvent in which the PEG intermediate has a low or substantially no solubility such as an ether (e.g., methyl tert-butyl ether, diethyl ether, etc.), isopropyl alcohol, or a similar organic solvent. Additional purification of the PEG intermediate may also be carried out using conventional purification techniques such as chromatography.

Following formation of the polymer amine intermediate, POLY-(X)_{0.1}—(R₂)_{0.1}—N(R₁)H, functionalization of the amine group to introduce a carbonyl functionality (˜C(Y)˜) may be carried out using a suitable reactant, such as for example, a phosgene such as phosgene, diphosgene, or triphosgene; other suitable reactants include metal carbonates/CO₂, dimethyl carbonate, N,N′-dissuccinimidyl carbonate (DSC), benzotriazolyl carbonate, and the like. Following introduction of the carbonyl group, for instance, to form the corresponding polymer carbamoyl halide (e.g., carbamoyl chloride) or other polymer carbamoyl intermediate, if desired, a reactive leaving group (Z) may be introduced to form a desired histidine-selective polymer reagent. Suitable reactive leaving groups include, for example, tetrazoles, isocyanates, isothiocyanates, N-hydroxysuccinimidyl esters, sulfonyl chloride, carbodiimide, acyl azide, carbonates, imidoesters, epoxides, fluorophenyl ester, anhydrides, benzotriazoles, and para-nitrophenyl carbonate, among others. Preferred leaving groups include, for example, substituted benzotriazole or substituted phenyl tetrazole, such as 5-[3,5-bis(trifluoromethyl)phenyl]-2H-tetrazole (BTMP).

Once prepared, the polymeric reagents can be isolated. Known methods can be used to isolate the polymeric reagent, such as, for example, precipitation. For instance, the solvent(s) can be removed or substantially removed from a crude product mixture containing the polymeric reagent, e.g., by evaporation under reduced pressure, to provide a crude product residue, followed by addition of a solvent suitable to effect precipitation of the polymeric reagent. Solvents that may be suitable for precipitating the polymeric reagent include, for example, ethers such as methyl-tert-butyl ether and diethyl ether, and alcohols such as isopropyl alcohol. Should additional purification be desired, the polymeric reagent (and, if desired, polymeric intermediates leading to the polymeric reagent) may be further purified using standard art-known purification methods, such as, for example, chromatography. Suitable chromatographic methods include, e.g., size exclusion chromatography, ion-exchange chromatography, normal phase, and reverse-phase chromatography.

Illustrative syntheses, including reagents, reaction conditions, solvents, temperature, isolation and purification techniques, and the like are provided in Examples 1-9.

### Conjugates

The present disclosure also includes conjugates obtainable/obtained by reacting a polymer reagent of Formula (I) with an active agent such as a biologically active agent or surface comprising one or more amino groups under conditions effective to promote conjugation between the one or more amino groups of the active agent and the polymer reagent. In preferred embodiments, the conjugate is obtainable/obtained by reacting a polymer reagent of Formula (I) with an active agent comprising one or more histidine residues comprising an amino group (“histidine amino group”), such that the linkage between the water-soluble polymer reagent and the active agent comprises a urea-imidazolyl or a thiourea-imidazolyl moiety, and the imidazolyl group forms part of a histidine residue of the active agent.

Conjugates in accordance with the instant disclosure preferably correspond to Formula (II):

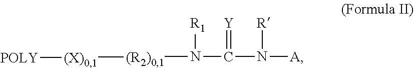

wherein POLY is a water-soluble polymer (e.g., a poly(ethylene glycol) or methoxypoly(ethylene glycol)); X is a linker moiety; R₁ is an organic radical and may form a nitrogen-containing heterocycle when taken together with R₂; R₂, when present, taken together with R₁ forms a nitrogen-containing heterocycle; Y is selected from O and S; R′ is H or an organic radical, and

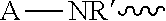

is an active agent comprising an amino group (NR′). Conjugate components POLY, X, R₂, R₁, and Y encompass each and every of the embodiments described above and elsewhere herein. The water-soluble polymer segment, POLY, may for example, have a weight average molecular weight in a range of from about 200 daltons to about 80,000 daltons, or from about 500 daltons to about 70,000 daltons, or from about 1,000 daltons to about 60,000 daltons, or from about 2,000 daltons to about 50,000 daltons, or from about 5,000 daltons to about 25,000 daltons, or from about 5,000 daltons to about 30,000 daltons, or from about 5,000 daltons to about 50,000 daltons, or from about 10,000 daltons to about 60,000 daltons, or from about 10,000 daltons to about 50,000 daltons, or from about 20,000 daltons to about 50,000 daltons, from about 20,000 daltons to about 40,000 daltons, or from about 20,000 daltons to about 80,000 daltons. In some more particular embodiments, POLY has a weight average molecular weight selected from the group consisting of 200 daltons, 300 daltons, 400 daltons, 500 daltons, 750 daltons, 1,000 daltons, 2,500 daltons, 3,000 daltons, 5,000 daltons, 7500 daltons, 10,000 daltons, 15,000 daltons, 20,000 daltons, 25,000 daltons, 30,000 daltons, 40,000 daltons, 50,000 daltons, 55,000 daltons, and 60,000 daltons. In preferred embodiments, ˜N-R′, when taken together with A, is an amine comprised within an imidazolyl ring of a histidine comprised in the active agent. The active agent is preferably a biologically active agent, including, for example, small molecules, peptides and proteins. Preferably, the active agent is a peptide or a protein comprising a histidine residue, which may be naturally-occurring or may be introduced into a target protein or peptide (by addition or substitution) using known protein engineering techniques, such as, for example, site-directed mutagenesis.

Thus, for conjugates formed by reaction with a histidine of an active agent, the conjugate will preferably correspond to the following formula:

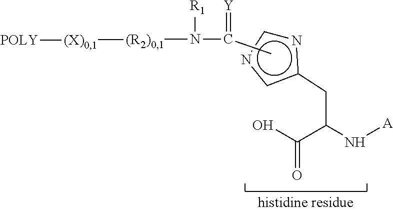

wherein reference to Formula (II), A-NR′ is an active agent comprising a histidine residue (shown in the above formula as a single amino acid, but with the understanding that the histidine may be comprised within a peptide or polypeptide chain), and POLY, X, R₂, R₁, and Y are as previously described. A formula more particularly depicting the histidine as comprised within a peptide or polypeptide (e.g., protein) is shown below:

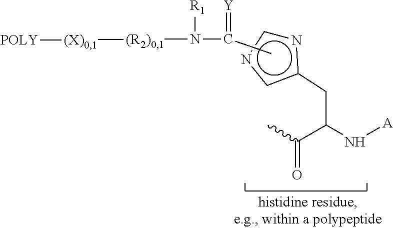

It is to be understood that conjugation can take place at either nitrogen on the histidine imidazole ring, and the foregoing structure is intended to encompass both isomers. Structures of each of the isomeric conjugates (with respect to position of covalent attachment on the histidine) are provided below; it is envisioned that covalent attachment as shown in the immediate structure below may be preferred, as the nitrogen atom (Nε2) in the imidazole ring appears to be less sterically hindered, however substitution at the other ring nitrogen, Nδ1 (see second structure below), may also occur.

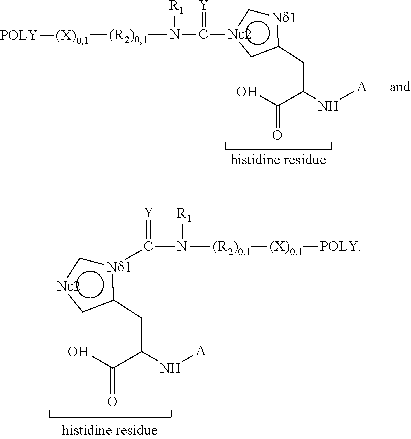

While the two structures above are intended to encompass an active agent comprising a histidine within a peptide or polypeptide chain (e.g., in a sequence of amino acids), for additional clarity, the following two structures more particularly indicate the presence of the histidine comprised within a longer sequence of amino acids such as in a peptide or polypeptide.

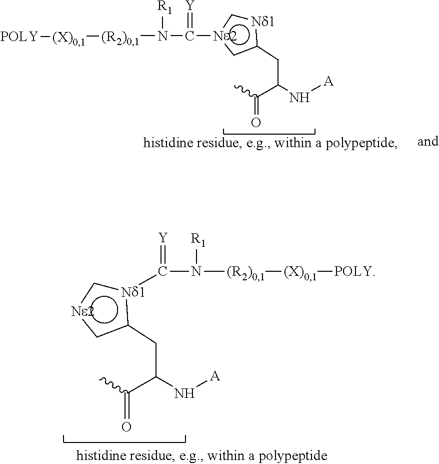

Illustrative conjugates include, for example,

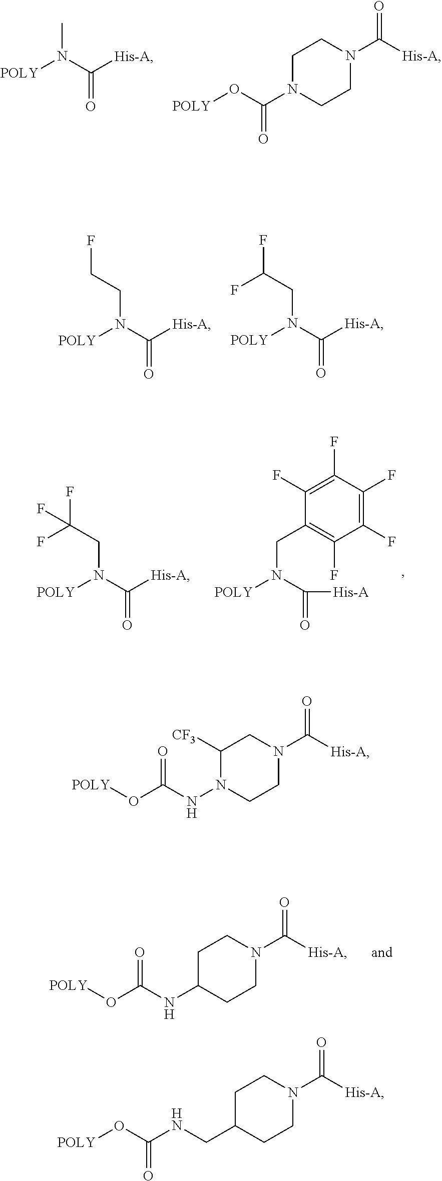

where “His” is a histidine residue of an active agent, A, and POLY is a water-soluble polymer as previously described.

For example, when POLY is a linear poly(ethylene glycol) polymer, a conjugate may have a structure selected from:

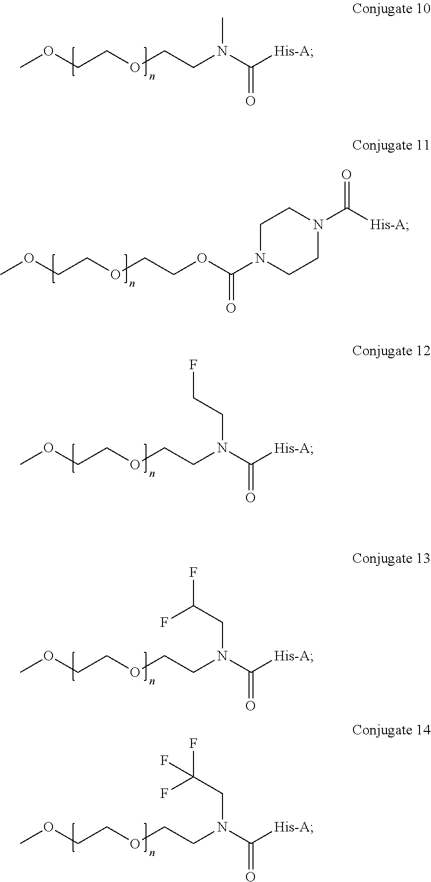

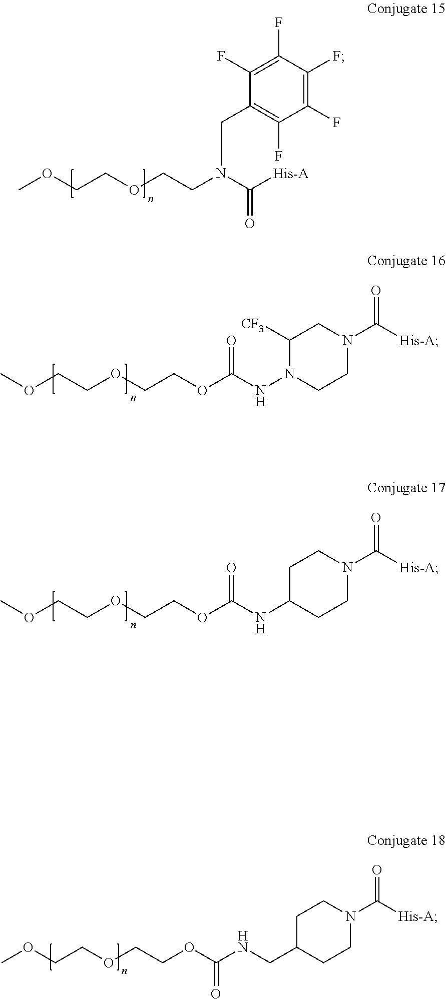

wherein (n) in each of Conjugates 11-18 is independently in a range of from about 2 to about 2,273 (including sub-ranges as previously described and particular values of (n)); His is a histidine residue, wherein attachment is at a histidine nitrogen atom; and A-His is an active agent, such as for example, a peptide or protein, comprising a histidine residue. The value of each (n) may, for example, fall within a range of from about 4 to about 1800; or from about 11 to about 1590; or from about 23 to about 1363; or from about 113 to about 568; of from about 113 to about 682; or from about 113 to about 1136; or from about 227 to about 1363; or from about 227 to about 1136; or from about 454 to about 1136; or from about 454 to about 909; or from about 454 to about 1818.

The polymeric reagents described herein are useful for conjugation to an active agent comprising an amino group. Thus, also provided is a method of preparing a conjugate of an active agent, the method comprising reacting a polymer reagent as provided herein with an active agent comprising one or more amino groups under conditions effective to promote conjugation between the one or more amino groups of the active agent and the polymer reagent. Preferably, the active agent comprises one or more histidine residues comprising an amino group (“histidine amino group”) that reacts with the polymer reagent under the reaction conditions employed to thereby form a polymer conjugate. As demonstrated in the accompanying examples, the novel polymer reagents provided herein are effective to selectively react with a histidine, e.g., such as in a peptide or protein.

Suitable conjugation conditions include those conditions of time, temperature, pH, reagent concentration, reactivity of the polymeric reagent, available functional groups in the active agent, solvent, and the like, conducive to effect conjugation between the polymeric reagent and an active agent while substantially maintaining protein structure (in the instance of the active agent being a protein). Moreover, and as illustrated in the accompanying examples, reaction conditions can be tailored to favor conjugation at histidines over lysines or the N-terminal of an active agent, e.g., a protein or peptide. As is known in the art, the particular conditions will depend upon, among other things, the active agent, the presence of other materials in the reaction mixture, and so forth. Reaction conditions for effecting conjugation in any particular case can be determined by one of ordinary skill in the art upon a reading of the instant disclosure, reference to the relevant literature, and/or through routine experimentation.

For example, to favor selective conjugation at a histidine residue (which are less abundant than lysines), conjugation can be carried out using a molar ratio of polymeric reagent to active agent (e.g., protein) of less than equimolar (e.g., from about 1:20 to about 1:10), or from about 1:5, or from about 1:2 molar ratio of polymer reagent to protein, to about equimolar (about 1:1), to having a small molar excess of polymer reagent relative to protein, e.g., from about 1.1-fold to a 20-fold molar excess of polymeric reagent. Further exemplary ratios of polymeric reagent to active agent (e.g., protein) are from about 1.1-fold to a 10-fold molar excess of polymeric reagent, or from about 1.1-fold to about a 5-fold molar excess of polymer reagent. Conjugation can be carried out over a wide range of temperatures, typically but not necessarily from about 0° C. to about 60° C., or from about 0° C. to about 40° C., or from about 0° C. to about 30° C., or from about 0° ° C. to about 10° C. Reactions can, for example, be carried out at ambient temperature. The conjugation reaction is typically carried out in a suitable solvent, e.g., an aqueous solvent for conjugation of proteins or peptides; for reactions with small molecules, organic solvents may be used. Conjugation may be carried out in a suitable buffer solution, such as for example, an aqueous solution containing a phosphate salt such as sodium phosphate, or sodium acetate, sodium carbonate, sodium bicarbonate, or the like. Concentration of the active agent, e.g., protein, typically ranges from about 0.1 mg/ml to about 5 mg/ml, or from about 0.5 mg/ml to about 2.5 mg/ml. The pH of the reaction is preferably slightly acidic, with pHs ranging from about 4.5 to about 6.8, or from about 4.8 to about 6.8. Depending upon the reactivity of the polymeric reagent and the accessibility of one or more histidine residues of the active agent, conjugation may be preferably carried out at a pH of from about 5.0 to about 6.8. While the reactivity of the polymeric reagent (depending upon X, R₂ and R₁) may vary, the histidine-reactivities of the reagents appear to generally follow a trend of increasing with increasing pH within a range of from about 4.5 to about 6.8, or more particularly, within a range of from about 5.0 to about 6.5 as illustrated in Table 1. Polymer reagents comprising an electron-withdrawing group within the spacer, ˜(X)_{0.1}—(R₂)_{0.1}-NR₁˜, appear to be more reactive than polymer reagents having the same or a similar structure but absent the electron-withdrawing group. Thus, depending upon the reactivity of the polymeric reagent employed in the conjugation reaction and the reaction variables and conditions, reaction times can range from less than an hour to several hours, in some cases up to twenty-four hours or even longer. The conjugation reaction is allowed to proceed until substantially no further conjugation occurs, which can generally be determined by monitoring the progress of the reaction over time. Progress of the reaction can be monitored by withdrawing aliquots from the reaction mixture at various time points and analyzing the reaction mixture by SDS-PAGE or MALDI-TOF mass spectrometry or any other suitable analytical method. Once a plateau is reached with respect to the amount of conjugate formed or the amount of unconjugated polymer remaining, the reaction is assumed to be complete.

Preferably, for active agents such as a protein containing a single histidine, e.g., a surface-accessible histidine, reaction with a polymer reagent of the instant disclosure under suitable reaction conditions will result in a product mixture comprising predominantly histidine-attached polymer-modified protein, that is to say, where a majority of conjugates formed are histidine-attached positional isomers, e.g., wherein at least about 50 mole percent of more of conjugates comprise the polymer (POLY) covalently attached (e.g., indirectly, via ˜(X)_{0.1}—(R₂)_{0.1}—NR₁—C(Y)˜), to a histidine residue of the active agent, or wherein at least about 60 mole percent or more of conjugates comprise the polymer (POLY) covalently attached to a histidine residue of the active agent. In some cases, a majority of conjugates formed are histidine-attached positional isomers, e.g., wherein at least about 75 mole percent or more of conjugates comprise the polymer (POLY) covalently attached (e.g., indirectly, via ˜(X)_{0.1}—(R₂)_{0.1}—NR₁—C(Y)˜), to a histidine residue of the active agent. In some instances, the composition will comprise conjugates wherein the water-soluble polymer POLY, is covalently attached to the active agent at only a histidine residue. For example, illustrative compositions include those wherein a majority of conjugates formed are histidine-attached positional isomers, e.g., wherein at least about 50 mole percent of more of conjugates comprise the polymer (POLY) covalently attached (e.g., indirectly, via ˜(X)_{0.1}—(R₂)_{0.1}—NR₁—C(Y)˜), only at a histidine residue of the active agent, or wherein at least about 60 mole percent or more of conjugates comprise the polymer (POLY) covalently attached only at a histidine residue of the active agent. In some cases, a majority of conjugates formed are histidine-attached positional isomers, e.g., wherein at least about 75 mole percent or more of conjugates comprise the polymer (POLY) covalently attached (e.g., indirectly, via ˜(X)_{0.1}—(R₂)_{0.1}—NR₁—C(Y)˜), only at a histidine residue of the active agent.

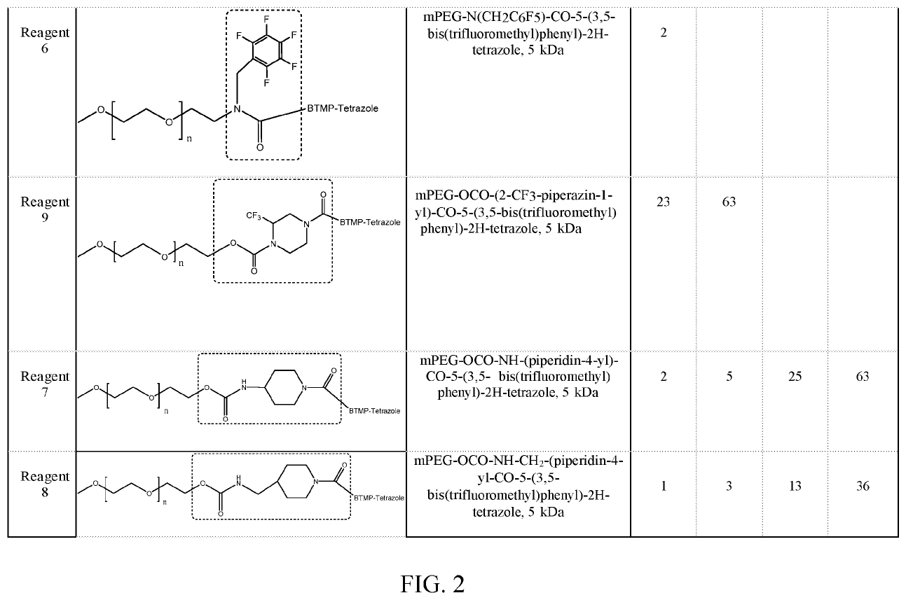

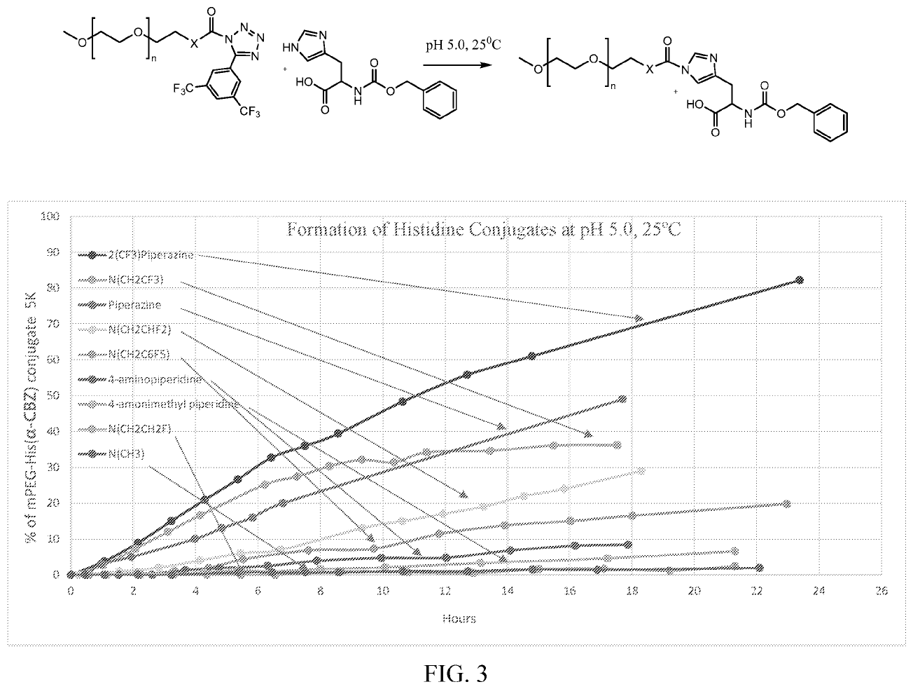

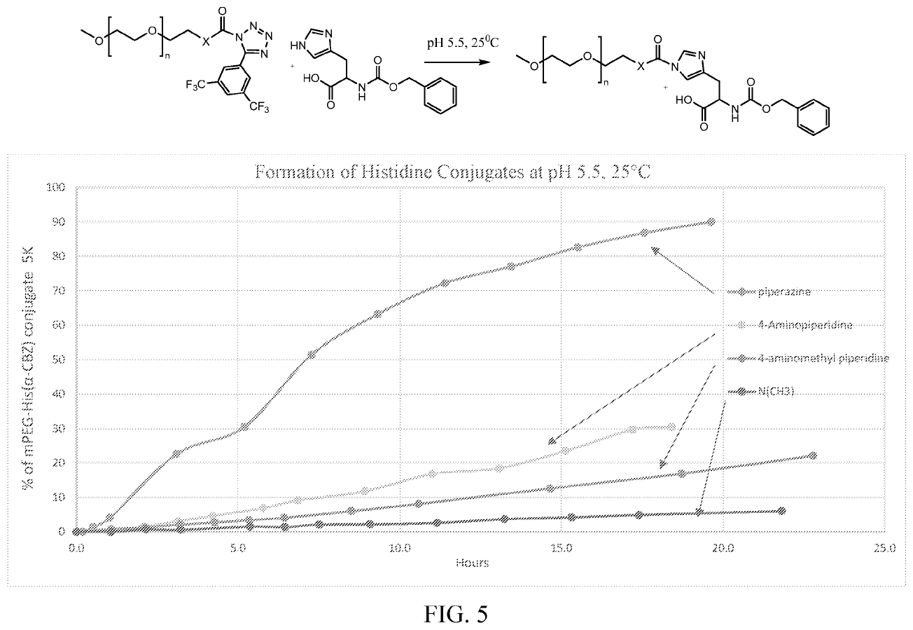

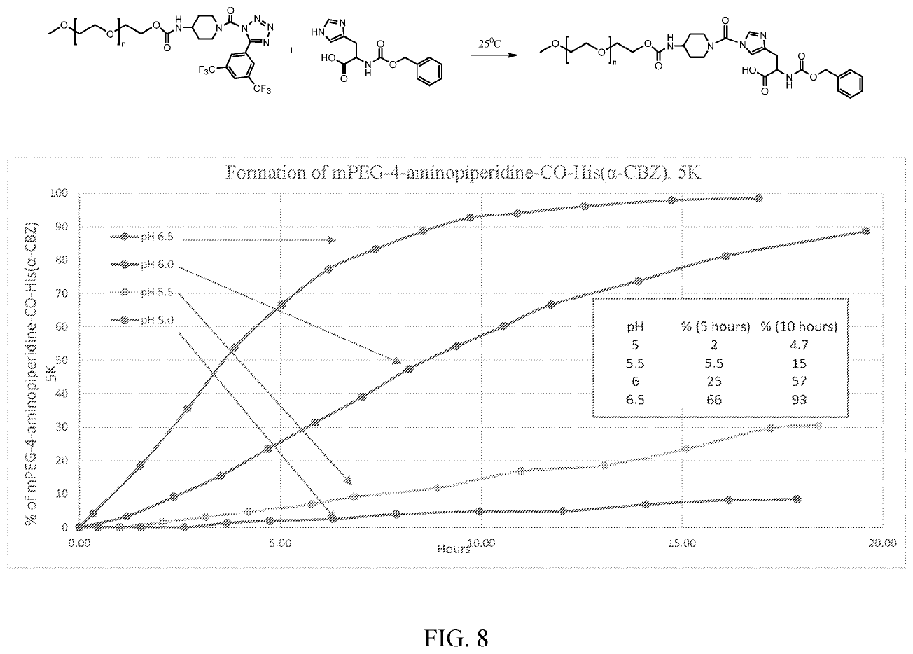

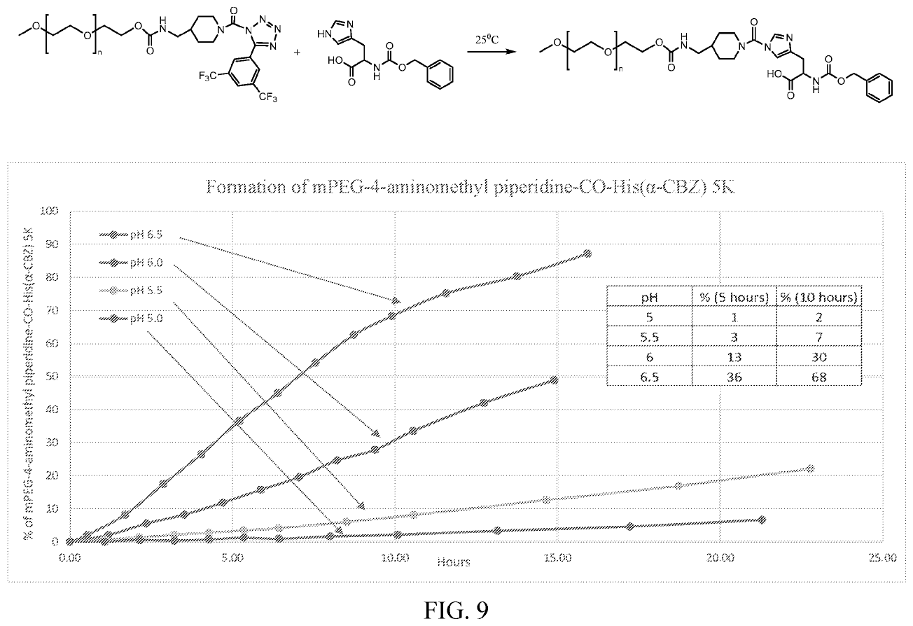

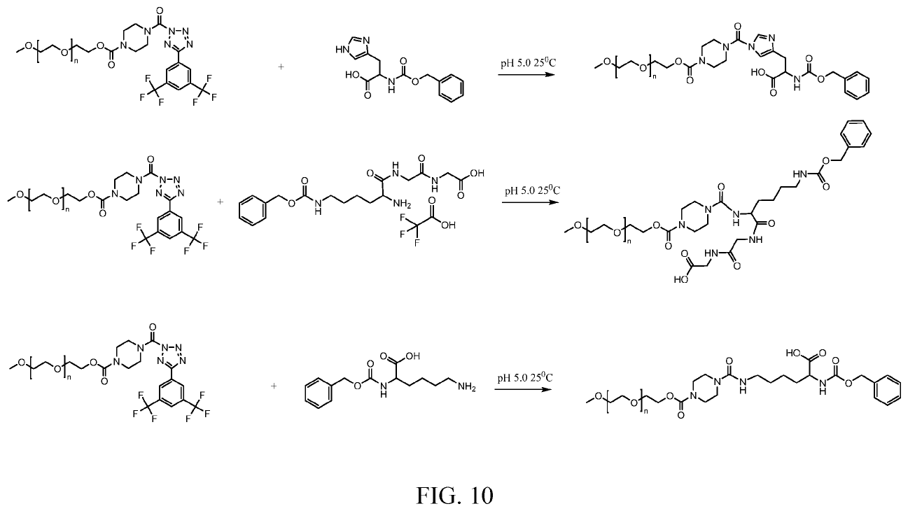

As shown in the accompanying examples and as previously described, the polymeric reagents provided herein are capable of reaction with histidines. See, for example, FIG. 2, which provides a table of illustrative histidine-reactive poly(ethylene glycol) reagents containing a variety of exemplary spacer groups intervening between the PEG moiety and the BTMP-tetrazole (or other suitable) leaving group as indicated by the dashed boxes (i.e., ˜(X)_{0.1}—(R₂)_{0.1}—NR₁—C(O)˜), and their reactivities based upon percent conjugate formed under different pH conditions (5.0, 5.5, and 6.5) at a reaction time of five hours, as described in Example 10. These data support the feature of the subject polymer reagents as being histidine-reactive, and further illustrate that the reactivity of the reagents can be effectively tailored by the selection of the components of the spacer moiety to achieve optimal reactivity suited for a given application and reaction conditions. The reactivities of the reagents appear to generally follow a trend of increasing with increasing pH under the given conditions. As an example, FIG. 3 is a plot showing percent of histidine-linked conjugate formed over time at pH 5.0 at 25° C. for illustrative histidine-selective poly(ethylene glycol) reagents containing a variety of amino groups (X) intervening between the PEG moiety and the illustrative ˜C(O)BTMP-tetrazole (or other suitable) leaving group as described in Example 10. Similarly, FIGS. 5, 6, and 7 are plots illustrating the percent of histidine-linked conjugate formed over time at 25° C. and at pH 5.5, 6.0, and 6.5, respectively, for illustrative histidine-selective poly(ethylene glycol) reagents containing a variety of amino groups (X) intervening between the PEG moiety and the ˜C(O)BTMP-tetrazole (or other suitable) leaving group as described in Example 10. See also the results shown in FIGS. 8 and 9. FIG. 8 illustrates the reactivity of mPEG-4-aminopiperidine-C(O)-5-(3,5-bis(trifluoromethyl)phenyl-2H-tetrazole, 5 kD, with the model compound, carboxybenzyl (CBZ)-histidine, at four different pHs (5.0, 5.5, 6.0, and 6.5). As described in Example 10, reactivity can be altered by, for example, changing the pH. As shown in the figure, after 10 hours, at pHs 5.0, 5.5, 6.0, and 6.5, the percent (%) conjugate formed was 4.7, 15, 57 and 93, respectively. Similarly, FIG. 9 illustrates the reactivity of mPEG-4-aminomethylpiperidine-C(O)-5-(3,5-bis(trifluoromethyl)phenyl-2H-tetrazole, 5 kD, with the model compound, carboxybenzyl (CBZ)-histidine, at four different pHs (5.0, 5.5, 6.0, and 6.5) as described in Example 10. As shown in the figure, after 10 hours, at pHs 5.0, 5.5, 6.0, and 6.5, the percent conjugate formed was 2.0, 7, 30 and 68, respectively. These data also illustrate the relatively lower reactivity of the mPEG-4-aminomethylpiperidine-C(O)-5-(3,5-bis(trifluoromethyl)phenyl-2H-tetrazole reagent in comparison to the mPEG-4-aminopiperidine-C(O)-5-(3,5-bis(trifluoromethyl)phenyl-2H-tetrazole reagent, wherein the two reagents differ in the absence or presence of a ˜CH₂˜ group interposed between the carbamate nitrogen and the 4-carbon of the piperidine ring, thereby illustrating the ability to tailor the selection of the polymeric reagent to the target active agent and desired application(s).

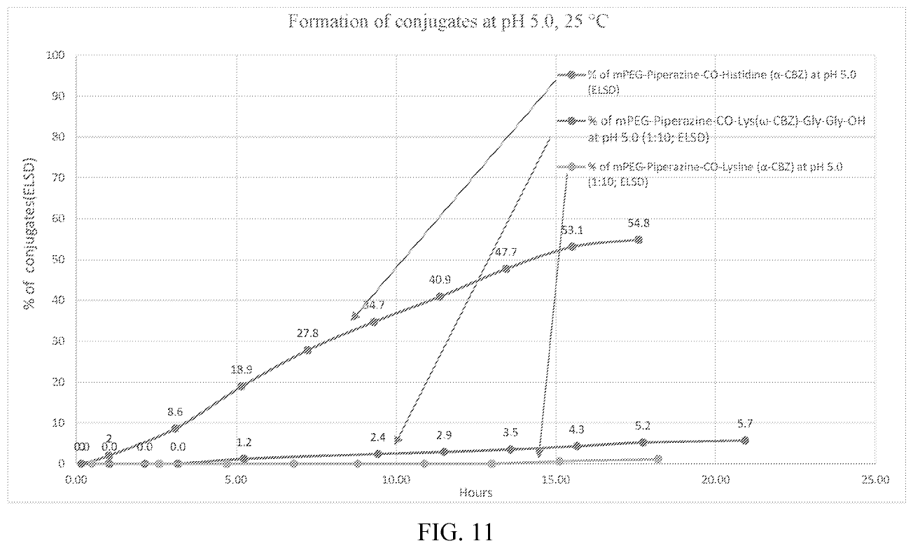

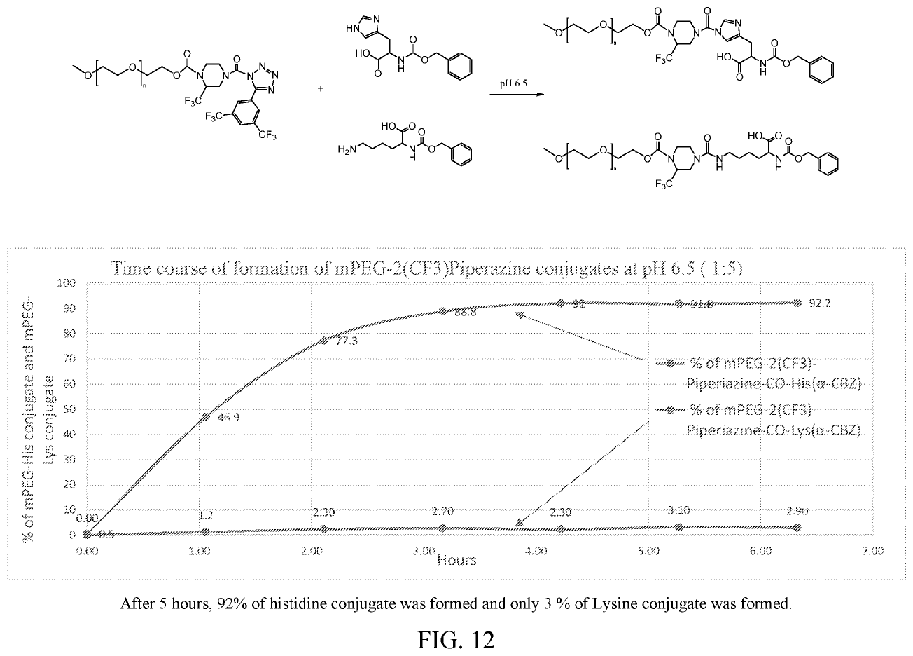

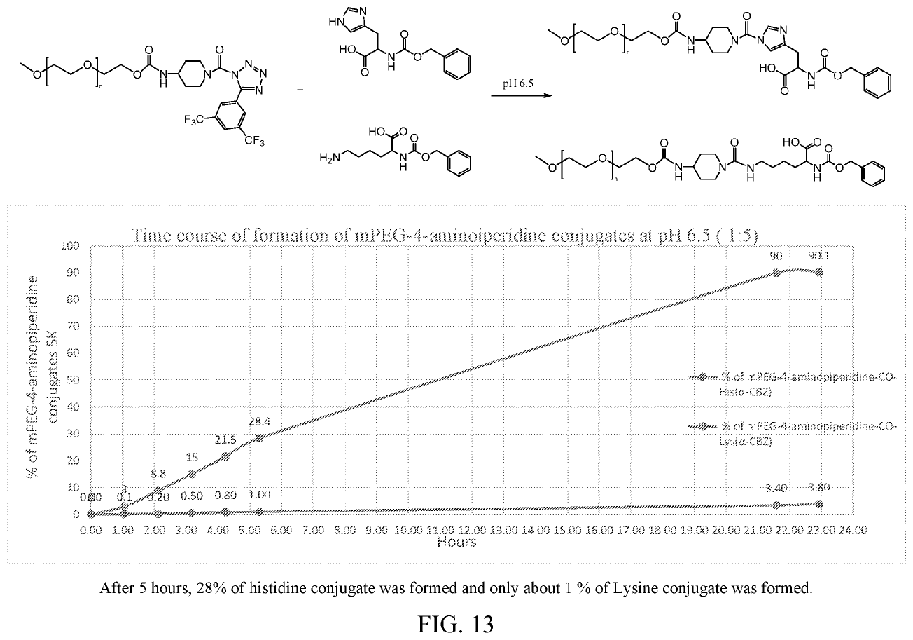

Reaction conditions for selective conjugation can be further optimized by one skilled in the art. Further supporting the histidine-selective feature of the polymeric reagents described herein, Examples 11 and 12 demonstrative the proclivity of illustrative Polymer Reagent 2 to selectively react with histidine over lysine in the model compounds, α-CBZ-histidine, α-CBZ-lysine, and the tri-peptide, w-CBZ-lysine-glycine-glycine-OH, under the reaction conditions employed. See, for example, the results summarized in Table 2. FIG. 11 demonstrates the selectivity of an illustrative PEG reagent as provided herein, Reagent 2, where R₂ when taken with —NR₁ forms piperazine, and where ˜(X)_{0.1}—(R₂)_{0.1}—NR₁—C(O)˜) is ˜O—C(O)-piperazine-C(O)˜, when reacted with different amino acid or oligopeptide targets: α-CBZ-His, α-CBZ-Lys, and ω-CBZ-Lys-Gly-Gly-OH at a molar ratio of 1:10, in phosphate buffer at 25° C. The results illustrate the notable selectivity of the reagents provided herein for histidine over lysine as described in Example 11. As additional evidence, FIG. 12 illustrates the selectivity of an illustrative PEG reagent, Reagent 9, where R₂ when taken with —NR₁ forms piperazine, and where ˜(X)_{0.1}—(R₂)_{0.1}—NR₁—C(O)˜) is ˜O—C(O)-piperazine-C(O)˜, when reacted with different amino acid or oligopeptide targets: α-CBZ-His, α-CBZ-Lys, and ω-CBZ-Lys-Gly-Gly-OH at a molar ratio of 1:10, in phosphate buffer at 25° C. The results further illustrate the striking selectivity of the reagents provided herein for histidine over lysine as described in Example 13. See also, FIG. 13, demonstrating the histidine selectivity of Reagent 7, where R₂ when taken with —NR₁ forms piperidine, and where ˜(X)_{0.1}—(R₂)_{0.1}—NR₁—C(O)˜) is ˜O—C(O)-NH-piperidine-C(O)˜, as described in detail in Example 13, providing further evidence of the histidine-selectivity of the polymer reagents provided herein.

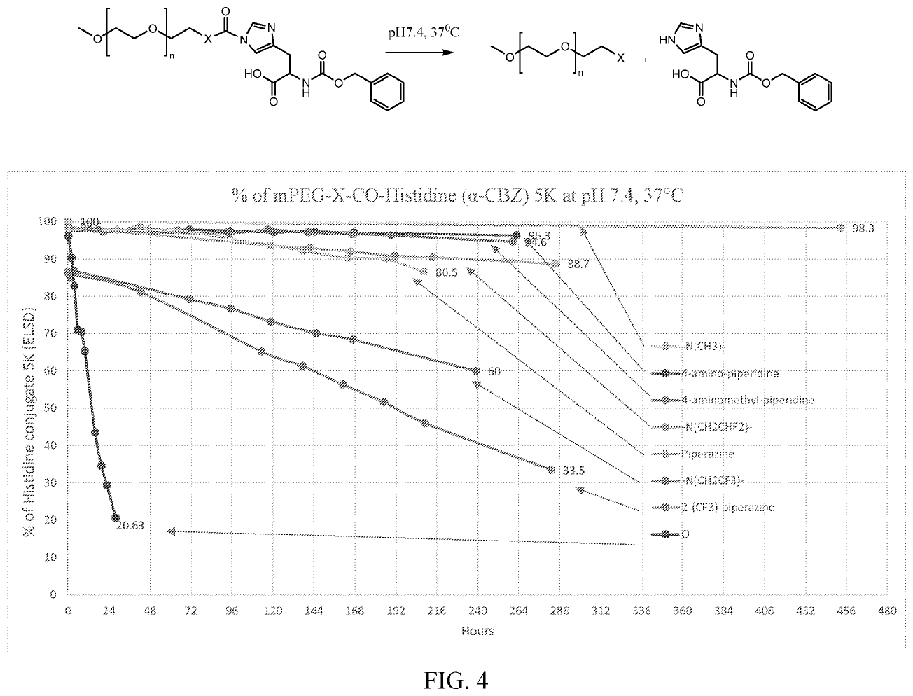

Another notable feature and advantage of histidine-conjugates formed by reaction with the instant polymer reagents is their hydrolytic stability, as demonstrated, for instance, in FIG. 4. As shown therein, by virtue of a plot showing the results of a hydrolytic stability study for exemplary histidine-linked poly(ethylene glycol) conjugates containing a variety of spacer moieties intervening between the PEG moiety and the covalent attachment to histidine (˜(X)_{0.1}—(R₂)_{0.1}—NR₁—C(O)-His), and as further described in Example 14, conjugates formed by reaction of a model histidine-comprising compound, α-CBZ-histidine, with exemplary histidine-selective polymer reagents as provided herein, are relatively stable in aqueous buffer at pHs up to about 8.0. The urea-linked conjugates that were evaluated and that demonstrated the highest degree of hydrolytic stability relative to the other conjugates, Conjugates 10, 17 and 18, are those wherein R₁ optionally in combination with R₂, when present, in reference to the general formula,

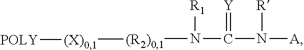

are methyl and piperidine. Conjugate 11, having a moderate degree of hydrolytic stability relative to the other conjugates tested, comprises a piperazine moiety (formed by R₁ and R₂, taken together in combination with N), in addition to linker X, ˜OC(O) ˜. Conjugate 14, similarly having a moderate degree of hydrolytic stability relative to the other conjugates tested, possesses as R₁, ˜CH₂CH₂CF₃. Finally, the conjugate showing the highest degree of hydrolysis under the test conditions, Conjugate 16, comprises a trifluoromethyl-substituted piperazine (formed by R₁ and R₂, taken together in combination with N) in addition to linker X, ˜OC(O) ˜. The hydrolytic stability of the resulting conjugates (at a given pH and temperature, such as, for example, under physiological conditions or conditions mimicking physiological conditions), is influenced by the selection of R₁, R₂ and X, where the presence of electron withdrawing groups appears to contribute to a greater degree of hydrolysis. Thus, conjugates as generally described herein, and comprising one or more electron-withdrawing groups or atoms within the overall spacer, ˜(X)_{0.1}—(R₂)_{0.1}-NR₁˜ are expected to exhibit a diminished hydrolytic stability when compared to conjugates absent such electron withdrawing moieties. Thus, polymer reagents (and thus the conjugates formed by reaction therewith) can be designed to possess optimal reactivity, selectivity and conjugate stability for reaction with a desired target molecule or surface by appropriate selection of polymer reagent components such as X, R₂ and R₁.

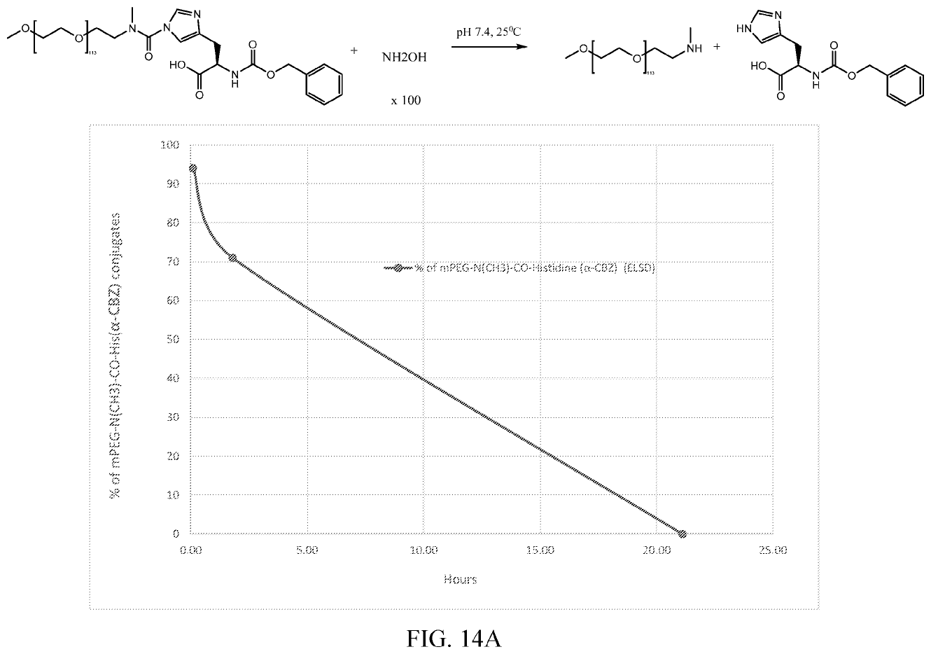

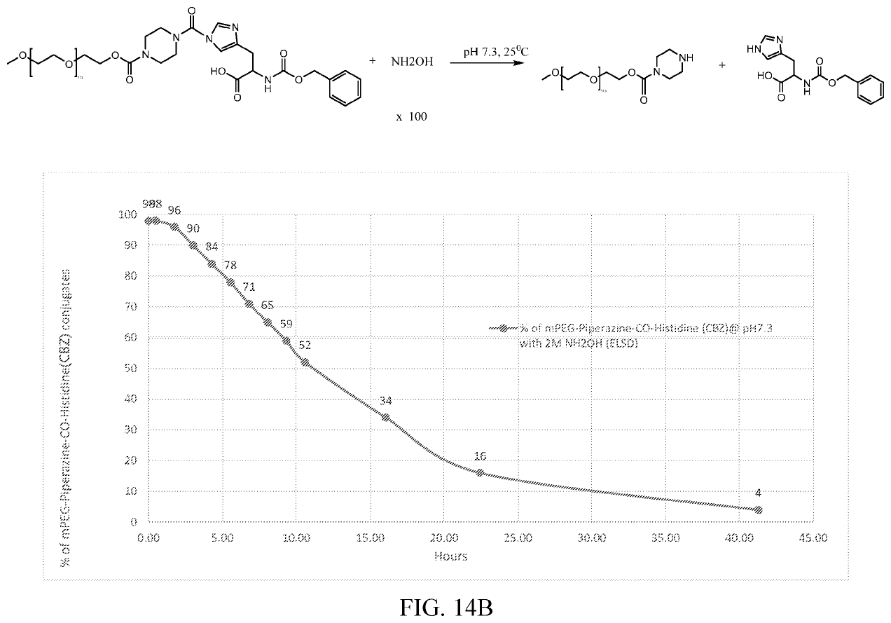

Histidine conjugates such as the illustrative conjugates tested, when treated with hydroxylamine, undergo a reverse reaction such that the unconjugated histidine compound is released. In contrast, hydroxylamine does not typically react with a lysine-linked conjugate to cleave the lysine-polymer linkage to thereby release the parent lysine compound. FIG. 14A is a plot illustrating reaction of a histidine conjugate (prepared by reaction of Reagent 1 with model compound, α-CBZ-His) with hydroxylamine (pH 7.4, 25° C.). The plot shows percent of mPEG-N(CH₃)—CO-His(α-CBZ) conjugate remaining over time. In this model reaction, by approximately 22 hours, 100% of the conjugate has disappeared with release of a-CBZ-His. Similarly, FIG. 14B illustrates the reaction of a different histidine conjugate (prepared by reaction of Reagent 2 with model compound, α-CBZ-His), with hydroxylamine (pH 7.3, 25° C.). The plot shows percent of mPEG-piperazine —CO-His(α-CBZ) conjugate remaining over time, where at approximately 42 hours, only 4% of the intact conjugate remained. Thus, these experiments further support the histidine-selectivity of the polymeric reagents provided herein, as evidenced by destruction of the conjugates formed in the presence of hydroxyl amine (which would not typically occur for a lysine-linked conjugate).

In instances in which it is desired to favor conjugation to epsilon amino groups (e.g., lysines) of a peptide or protein, the conjugation reaction may be carried out under basic conditions, for example, at pHs ranging from about 7.5 to about 10, or from about 8.0 to about 10.

Reaction times are typically determined by monitoring the progress of the reaction over time. Progress of a conjugation reaction can be monitored by withdrawing aliquots from the reaction mixture at various time points and analyzing the reaction mixture by RP-HPLC, SDS-PAGE or MALDI-TOF mass spectrometry or any other suitable analytical method. The product mixture may be further characterized using analytical methods such as chromatography, MALDI, capillary electrophoresis, and/or gel electrophoresis.

The resulting product mixture is then preferably purified to reduce the quantities or remove one or more of excess PEG or other reagents, unreacted active agent (e.g., protein or peptide), other possible side-products, or to separate polymer conjugates having differing numbers of water-soluble polymers covalently attached thereto (as applicable).

For example, the conjugate-containing reaction mixture can be purified to obtain/isolate different conjugated species or provide a composition further enriched in a desired conjugate species, e.g., histidine-linked conjugates.

If desired, conjugates having different molecular weights can be isolated using gel filtration chromatography and/or ion exchange chromatography. For example, gel filtration chromatography may be used to separate conjugated species having different numbers of water-soluble polymer to active agents ratios (e.g., 1-mer, 2-mer, 3-mer, and so forth, wherein “1-mer” indicates 1 water-soluble polymer covalently attached to an active agent (e.g., at a histidine residue), and so forth, on the basis of their differing molecular weights.

While gel filtration chromatography can be used to separate unreacted polymeric reagent and conjugates having different molecular weights, this approach is generally ineffective for separating positional isoforms having different attachment sites to a protein or other active molecule. Gel filtration columns suitable for carrying out this type of separation include Superdex™ and Sephadex™ columns available from GE Healthcare (Buckinghamshire, UK). Selection of a particular column will depend upon the desired fractionation range desired. Elution is generally carried out using a suitable buffer, such as phosphate, acetate, or the like. The collected fractions may be analyzed by a number of different methods, for example, (i) absorbance at 280 nm for protein content, (ii) dye-based protein analysis using bovine serum albumin (BSA) as a standard, (iii) iodine testing for PEG content (Sims et al. (1980) Anal. Biochem, 107:60-63), (iv) sodium dodecyl sulfate polyacrylamide gel electrophoresis (SDS PAGE), followed by staining with barium iodide, and (v) high performance liquid chromatography (HPLC).

Alternatively, ion exchange chromatography can be used to carry out a separation/purification as described above. For example, cation exchange chromatography using a suitable buffer for elution may be employed to provide a purified conjugate composition. Cation exchange columns and suitable supports are available from various suppliers including Bio-Rad, Thermo Fisher, and GE Healthcare.

If desired, separation of positional isoforms may be carried out by reverse phase chromatography using a reverse phase-high performance liquid chromatography (RP-HPLC) using a suitable column (e.g., a C18 column or C3 column, available commercially from companies such as Agilent or Vydac) or by ion exchange chromatography using an ion exchange column, e.g., a Sepharose™ ion exchange column available from GE Healthcare. Such approaches can be used to separate positional isomers having the same molecular weight (i.e., positional isoforms differing in attachment site to a protein, e.g., histidine versus lysines).

The resulting conjugates, and in particular, histidine-linked conjugates, are stable over a broad range of pHs, including physiological pH (see, e.g., FIG. 4), thereby allowing facile chromatographic purification. See, for example, the results described in Example 14, wherein the hydrolytic stability of histidine-linked poly(ethylene glycol) conjugates containing a variety of spacer moieties intervening between the PEG moiety and a covalently attached histidine (of model compound, α-CBZ-histidine), was evaluated.

The present disclosure also includes pharmaceutical preparations comprising a conjugate as provided herein in combination with a pharmaceutical excipient. Generally, the conjugate itself will be in a solid form (e.g., a precipitate), which can be combined with one or more suitable pharmaceutical excipients that can be in either solid or liquid form.

Exemplary excipients include, without limitation, those selected from the group consisting of carbohydrates, inorganic salts, antimicrobial agents, antioxidants, surfactants, buffers, acids, bases, and combinations thereof.

The pharmaceutical preparations encompass all types of formulations and in particular, those that are suited for injection, e.g., powders that can be reconstituted as well as suspensions and solutions. The amount of the conjugate (i.e., the conjugate formed between the active agent and the polymer described herein) in the composition will vary depending on a number of factors, but will optimally be a therapeutically effective dose when the composition is stored in a unit dose container (e.g., a vial). In addition, the pharmaceutical preparation can be housed in a syringe. A therapeutically effective dose can be determined experimentally by repeated administration of increasing amounts of the conjugate in order to determine which amount produces a clinically desired endpoint.

The amount of any individual excipient in the composition will vary depending on the activity of the excipient and particular needs of the composition. Typically, the optimal amount of any individual excipient is determined through routine experimentation, i.e., by preparing compositions containing varying amounts of the excipient (ranging from low to high), examining the stability and other parameters, and then determining the range at which optimal performance is attained with no significant adverse effects.

Generally, however, the excipient will be present in the composition in an amount of about 1% to about 99% by weight, preferably from about 5%-98% by weight, more preferably from about 15-95% by weight of the excipient.

These foregoing pharmaceutical excipients along with other excipients are described in “Remington: The Science & Practice of Pharmacy”, 23^{rd} ed., A. Adejare, Academic Press, (2020), the “Physician's Desk Reference”, PDR, LLC., online version (2021), and Kibbe, A. H., Handbook of Pharmaceutical Excipients, 9th Edition, Eds. Sheskey, P. J., Hancock, B. C., Moss, G. P., Goldfarb, D. J., The Pharmaceutical Press (2020).

The pharmaceutical preparations of the present disclosure are typically, although not necessarily, administered via injection and are therefore generally liquid solutions or suspensions immediately prior to administration. The pharmaceutical preparation can also take other forms such as syrups, creams, ointments, tablets, powders, and the like. Other modes of administration are also included, such as pulmonary, rectal, transdermal, transmucosal, oral, intrathecal, subcutaneous, intra-arterial, and so forth.

As previously described, the conjugates can be administered parenterally by intravenous injection, or less preferably by intramuscular or by subcutaneous injection. Suitable formulation types for parenteral administration include ready-for-injection solutions, dry powders for combination with a solvent prior to use, suspensions ready for injection, dry insoluble compositions for combination with a vehicle prior to use, and emulsions and liquid concentrates for dilution prior to administration, among others.

The examples that follow illustrate (i) new histidine-selective water-soluble polymer reagents, (ii) methods for synthesizing such reagents; (iii) preferred reactivity/selectivity of the polymer reagents with histidine over lysine, (iv) conjugates formed by reaction with the subject reagents, and (v) the hydrolytic stability of the corresponding conjugates under physiological conditions, among other things.

All articles, books, patents, patent publications and other publications referenced herein are incorporated by reference in their entireties. In the event of an inconsistency between the teachings of this specification and the art incorporated by reference, the meaning of the teachings and definitions in this specification shall prevail (particularly with respect to terms used in the claims appended herein). For example, where the present application and a publication incorporated by reference defines the same term differently, the definition of the term shall be preserved within the teachings of the document from which the definition is located.

### EXAMPLES

It is to be understood that the foregoing description as well as the examples that follow are intended to illustrate and not limit the scope of the invention(s) provided herein. Other aspects, advantages and modifications within the scope of the invention will be apparent to those skilled in the art to which the invention pertains.

### Materials and Methods

This disclosure will, unless otherwise indicated, utilize conventional techniques of organic synthesis and the like, which are understood by one of ordinary skill in the art and are explained in the literature. In the following examples, efforts have been made to ensure accuracy with respect to numbers used (e.g. amounts, temperatures, and so forth), but some experimental error and deviation should be accounted for. Unless otherwise indicated, temperature is in degrees celsius and pressure is at or near atmospheric pressure at sea level.

Illustrative syntheses, including reagents, reaction conditions, solvents, temperatures, isolation and purification techniques and the like are provided in the accompanying examples; these examples are meant to be illustrative rather than limiting and are provided as guidance for preparing a variety of polymeric reagents in accordance with Formula (I) in light of the instant disclosure.

It is to be understood that for histidine-linked conjugates, conjugation can take place at either nitrogen on the histidine imidazole ring (N&2 or N81), and that the structures depicted are intended to encompass both isomers, even if both isomers are not explicitly shown.

Except for PEG reagents, all reagents were obtained commercially unless otherwise indicated. All PEG raw materials were supplied by Nektar Therapeutics in Huntsville, AL. All NMR data generated at Nektar Therapeutics were obtained using either 300 or 400 MHz NMR systems manufactured by Bruker (Billerica, MA).

### Abbreviations:

ACN acetonitrile
anh anhydrous
BHT butylated hydroxytoluene
di-BTC dibenzotriazoyl carbonate
DCM dichloromethane
DCC N,N′-dicyclohexyl carbodiimide
DMAP 4-dimethylaminopyridine
DMF dimethylformamide
EDAC HCl 1-ethyl-3-(3′-dimethylaminopropyl)carbodiimide, HCl
HOBT hydroxybenzotriazole
IPA isopropyl alcohol
MTBE methyl-tert-butyl ether
NHS N-hydroxysuccinimide
Pyr pyridine
RB round-bottomed
RT room temperature, 20 to 25° C.
SC succinimidyl carbonate
THF tetrahydrofuran

### Example 1

SYNTHESIS OF mPEG-N(CH₃)—CO-5-(3,5-BIS(TRIFLUOROMETHYL)PHENYL)-2H-TETRAZOLE, 5 kDa (REAGENT 1) 1.1. Preparation of mPEG-mesylate, 5 kDa

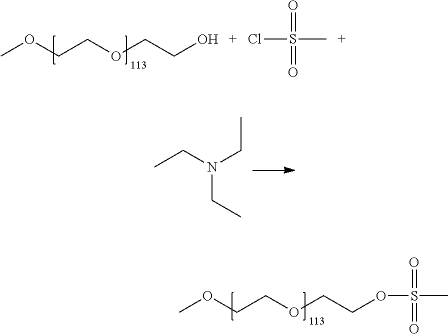

mPEG-OH 5 kDa (30 g, 6 mmol) was dissolved in 300 mL of toluene and then the solvent was distilled off to dryness. The residue was dissolved in 300 mL of anhydrous dichloromethane (DCM), and triethylamine (1.25 mL, 9 mmol) was added. The mixture was stirred for 5 minutes under nitrogen flow and methanesulfonyl chloride (0.51 mL, 6.6 mmol) was added dropwise with vigorous stirring. The reaction mixture was stirred at ambient temperature overnight under nitrogen atmosphere.

Next, the solvent was evaporated, and the product was precipitated by addition of 600 mL of isopropyl alcohol (IPA). The precipitate was collected by vacuum filtration and dried under reduced pressure. Yield: 29.2 g; ¹H NMR in CDCL₃ showed full substitution. 1H NMR (CDCl₃): δ, ppm 4.38 (m, —CH₂-OMs, 2H), 3.65 (bs, PEG backbone), 3.38 (s, —OCH₃, 3H), 3.09 ppm (s, —CH₃ mesylate, 3H).

1.2. Preparation of mPEG-NH(CH₃), 5 kDa

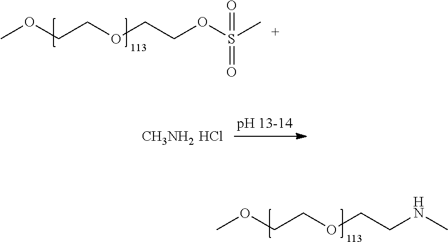

Methylamine hydrochloride (19.58 g, 290 mmol) was dissolved in 180 mL of DI water and the solution was cooled to 0-5° C. Sodium hydroxide (11.6 g, 290 mmol) dissolved in 120 mL of DI water was added into the methylamine hydrochloride solution. The pH was then adjusted to 13-14 by addition 1.0 M NaOH. After the temperature was elevated to ambient temperature, mPEG-Mesylate 5 kDa (29 g, 5.8 mmol) was added. The reaction mixture was stirred at ambient temperature for two days.

Next, NaCl (30 g) was added and the product was extracted with DCM (100 mL, 50 mL X 2). The extract was dried with anhydrous MgSO₄, filtered and the solvent was distilled off. The residue was precipitated with IPA/diethyl ether (1:1; 250 mL). The precipitate was collected and dried under vacuum for overnight. Yield: 27 g; Purity ˜ 90% (1H NMR in D₂O).

The crude product was purified by cation exchange chromatography using a POROS 50HS column (Thermo Fisher). Yield 23.5 g. Purity 100% by 1H NMR in D₂O. ¹H NMR (D₂O): δ (ppm) 3.73 (bs, PEG backbone), 3.41 (s, —OCH₃, 3H), 2.84 (t, —CH₂—N—, 2H), 2.43 (s, CH₃N—, 3H).

1.3. Preparation of mPEG-N(CH₃)—CO—Cl, 5 kDa

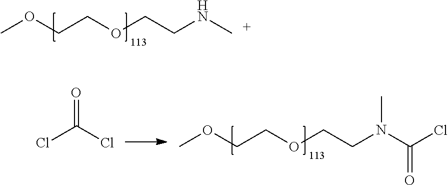

mPEG-methylamine 5 kDa (3.0 g, 0.5900 mmol) was dissolved in 50 mL of toluene in a 250 mL round- bottom flask. The solvent was distilled off to dryness under reduced pressure (rotary evaporator). The dried residue was dissolved in 10 mL of anhydrous DCM. 40 mL of anhydrous toluene was added followed by addition of 9.03 mL (1.78 mmol) of phosgene solution in toluene. Next the reaction mixture was stirred at ambient temperature overnight.

The solvent was distilled off to dryness under reduced pressure and the residue (crude mPEG-N(CH₃)—CO—C1 5 kDa) was precipitated by addition of 50 mL of methyl tert-butyl ether (MTBE). The precipitate was collected by vacuum filtration and dried overnight under reduced pressure to afford 2.85 g of product. Purity by 1H NMR in CDCl₃ 95.7%. 1H NMR (CDCl₃): δ (ppm) 3.64 (bs, PEG backbone), 3.38 (s, —OCH₃, 3H), 3.21 and 3.12 (s, CH₃N—, 3H).

1.4. Preparation of mPEG-N(CH₃)—CO-5-(3,5-bis(trifluoromethyl)phenyl)-2H-tetrazole, 5 kDa

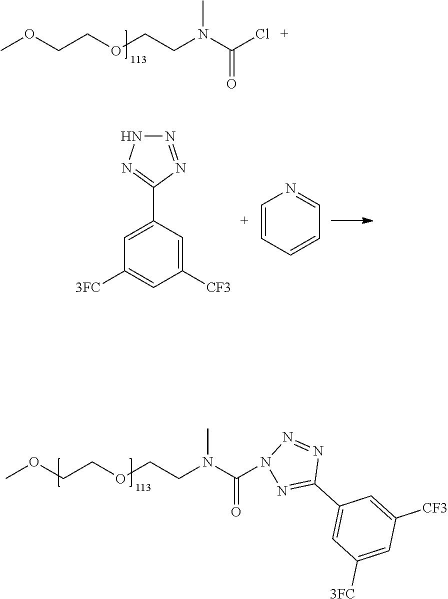

mPEG-N(CH₃)—CO—Cl (200 mg, 0.0400 mmol) was dissolved in the acetonitrile solution (1.2 mL) containing 5-(3,5-bis(trifluoromethyl)phenyl)-2H-tetrazole (0.1200 mmol). Pyridine (0.48 mL, 6 mmol) was added. The mixture was stirred at ambient temperature overnight.

Next, the solvent was distilled off under reduced pressure and the product was precipitated by addition of 20 mL MTBE. The precipitate was collected and dried under vacuum to afford 172 mg of the desired product. 1H NMR (CDCl₃): δ (ppm) 8.72 (s, Ar, 2H), 8.03 (s, Ar, 1H), 3.64 (bs, PEG backbone), 3.38 (s, —OCH₃, 3H), 3.36 and 3.28 (s, CH₃N—, 3H).

The above exemplary syntheses may be suitably carried out using PEG starting materials of a variety of molecular weights as described herein.

### Example 2

### SYNTHESIS OF mPEG-PIPERAZINE-CO-5-(3,5-BIS(TRIFLUOROMETHYL)PHENYL)-2H-TETRAZOLE, 5 kDa (REAGENT 2)

2.1. Preparation of mPEG-Succinimidyl Carbonate (mPEG-SC) 5 kDa

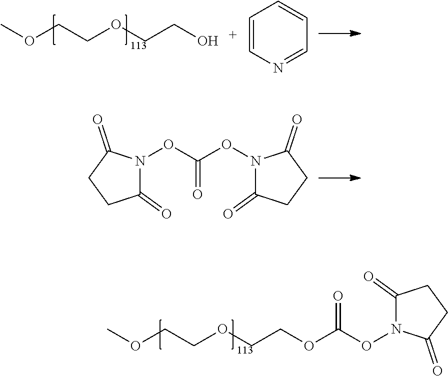

mPEG-OH, 5 kDa (20 g, 4 mmol) was dissolved in 100 mL of chloroform and the solvent was evaporated to dryness. The residue was dissolved in 150 mL of anhydrous acetonitrile. N, N′-disuccinimidyl carbonate (DSC) (2.05 g, 8 mmol) was then added. The mixture was stirred for 15 minutes, and pyridine (1.61 mL, 20 mmol) was added. The reaction mixture was stirred at ambient temperature for overnight.

A sample of the reaction mixture was analyzed by NMR showing 90% substitution, after which an additional 30% more of reagents was added (DSC: 0.62 g; pyridine: 0.48 mL). The reaction mixture was stirred at ambient temperature for 6 hours. The solvent was then evaporated to dryness, and the residue precipitated by addition of 300 mL of a mixture of IPA/ethyl ether (7:3). The precipitate was collected and dried under vacuum overnight.

The crude product was dissolved in 40 mL of DMF (with warming) and precipitated by addition of 160 mL of MTBE. The precipitate was collected and washed with 100 mL of diethyl ether and dried under vacuum for 4 hours to afford 17.3 g of product.

17.3 g of product was dissolved in 20 mL of DCM, and precipitated with IPA (200 mL) at 0-5° C. The precipitate was stirred at ambient temperature for 15 minutes. The precipitate was collected and dried under vacuum overnight. Yield: 16.2 g, Purity: ˜ 100%; no DCS and free NHS (NMR). 1H NMR (CDCl₃): δ (ppm) 4.46 (m, —CH₂—O—NHS, 2H), 3.64 (bs, PEG backbone), 3.38 (s, —OCH₃, 3H), 2.84 (s, —CH₂CH₂—, NHS, 4H).

2.2 Preparation of mPEG-Piperazine, 5 kDa

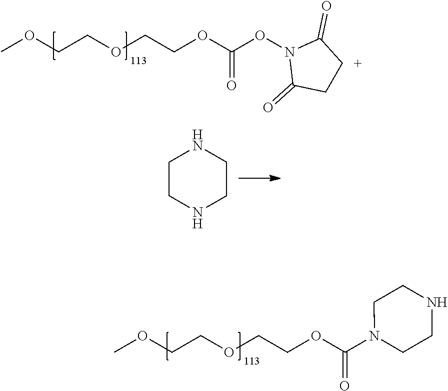

Piperazine (689 mg, 8.0 mmol) was dissolved in 50 mL of DCM. Next, mPEG-SC, 5 kDa (2.0 g, 0.40 mmol) was added to the solution. The mixture was stirred at ambient temperature overnight. The reaction mixture was filtered, the DCM was evaporated, and the residue was precipitated by addition of 40 mL of IPA. The precipitate was collected, washed with 10 mL of MTBE and dried under vacuum overnight. Yield: 1.89 g; End group substitution 93%; (HPLC).

The crude product was purified by cation exchange chromatography using a POROS 50 HS column (Thermo Fisher). Yield: 1.28 g; HPLC showed 100% of substitution.

1H NMR (D₂₀): δ (ppm) 4.23 (m, —CH₂—O(C═O)—N—, 2H), 3.68 (bs, PEG backbone), 3.36 (s, —OCH₃, 3H), 2.80 (m, —CH₂—NH—CH₂—, piperazine, 4H).

2.3 Preparation of mPEG-Piperazine-CO—Cl, 5 kDa

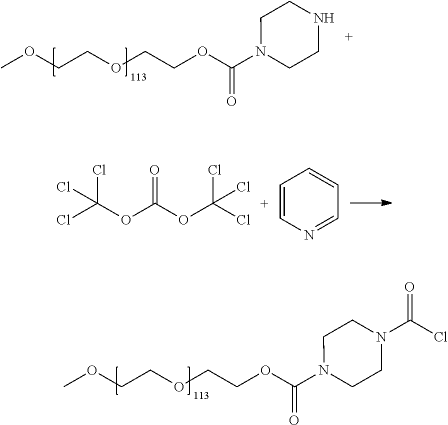

mPEG-Piperazine 5 kDa (1.28 g, 0.2600 mmol) was dissolved in 20 mL of toluene and the solvent was evaporated to dryness. The residue was dissolved in 10 mL of anhydrous DCM. Pyridine (0.03 mL, 0.3800 mmol) was added to PEG solution, and the mixture was cooled to 0-5° C. under a nitrogen atmosphere and treated with a solution of triphosgene (37.98 mg, 0.1300 mmol) in anhydrous DCM (10 mL). After addition of the triphosgene solution, the solution was stirred at 0° C. for one hour, then at ambient temperature overnight.

The solvent was distilled off under reduced pressure and 50 mL of MTBE was added. The resulting precipitate was filtered off and dried under vacuum to afford 1.25 g of the desired product. 1H NMR (CDCl₃): δ (ppm) 4.26 (m, —CH₂—O(C═O)—N, 2H), 3.63 (bs, PEG backbone), 3.37 (s, —OCH₃, 3H).

2.4 Preparation of mPEG-Piperazine-CO-5-(3,5-bis(trifluoromethyl)phenyl)-2H-tetrazole, 5 kDa

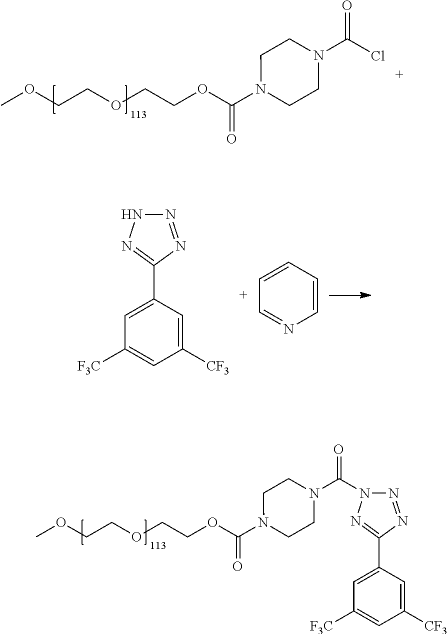

mPEG-Piperazine-CO—Cl, 5 kDa (500 mg, 0.10 mmol) was dissolved in the acetonitrile solution of 5-(3,5-bis(trifluoromethyl)phenyl)-2H-tetrazole (3 mL, 0.30 mmol). Pyridine (1.2 mL, 15.0 mmol) was added. The mixture was stirred at ambient temperature for overnight.

An aliquot of the mixture was checked by HPLC and showed no remaining starting material. Solvent was distilled off under reduced pressure and the residue was dissolved in 50 mL of DCM. The solution was washed with a solution of 10% H₂NaPO₄+5% of NaCl (10 mL×2), dried with MgSO₄, filtered, and the solvent was evaporated to dryness. The crude product was precipitated by addition of 40 mL of IPA. The precipitate was collected, washed with 10 mL of MTBE and dried under vacuum for three hours to afford 430 mg of the desired product. 1H NMR (CDCl₃): δ (ppm) 8.71 (s, Ar, 2H), 8.04 (s, Ar, 1H), 4.30 (m, —CH₂—(C═O)—N—), 3.65 (bs, PEG backbone), 3.38 (s, —OCH₃, 3H).

The above exemplary syntheses may be suitably carried out using PEG starting materials of a variety of molecular weights as described herein.

### Example 3

### SYNTHESIS OF MPEG-N(CH₂CH₂F)—CO-5-(3,5-BIS(TRIFLUOROMETHYL)PHENYL)-2H-TETRAZOLE, 5 KDA (REAGENT 3)

3.1 Preparation of mPEG-NH(CH₂CH₂F), 5 kDa

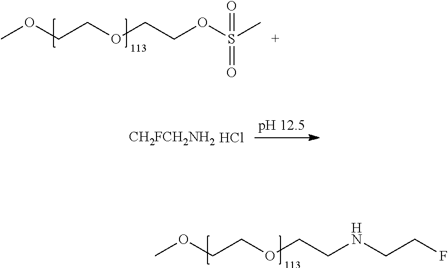

2-Fluoroethylamine hydrochloride (5 g, 50 mmol) was dissolved in DI water (100 mL), followed by pH adjustment with 1N NaOH to pH 12.5, and addition of mPEG-mesylate 5 kDa (10 g, 2 mmol, prepared by the procedure described in Example 1.1). The mixture was stirred at 20° ° C. for 63 h. NaCl was then added in an amount sufficient to form 10% NaCl in the solution, followed by extraction with DCM (50 mL×3). The extracts were combined and dried over MgSO₄. The solid was filtered off and the filtrate was concentrated to dryness. The residue was precipitated from MTBE (50 mL). The powder was collected by filtration, washed with IPA (30 mL) once, followed by washing with MTBE containing 300 ppm of BHT twice (20 mL×2). The solid was dried in vacuo for 12 hours. Yield: 9.7 g.

The crude product was purified by cation exchange chromatography using a POROS 50HS column. Yield: 2.45 g. Purity by HPLC: 96.3%. 1H-NMR (DMSO-d₆): δ (ppm) 4.50(t, —CH₂—F, 1H), 4.38(t, —CH₂—F, 1H), 3.50 (bs, PEG backbone), 3.24 (s, —OCH₃, 3H), 2.83 (t, —N—CH₂—CH₂F, 1H), 2.76 (t, —N—CH₂—CH₂F, 1H), 2.68 (t, —CH₂—NH—, 2H).

3.2 Preparation of mPEG-N(CH₂CH₂F)—CO—Cl, 5 kDa

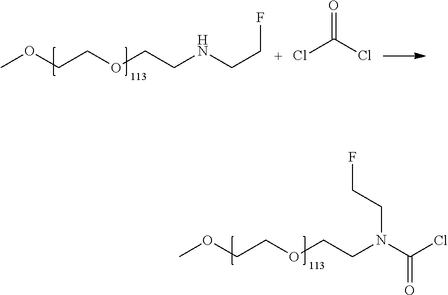

mPEG-NH(CH₂CH₂F), 5 kDa (1.0 g, 0.2 mmol) was dissolved in 30 mL of toluene in a round-bottom flask (100 mL). The solvent was distilled off to dryness under reduced pressure. The residue was dissolved in a mixture of anhydrous DCM (15 mL) and anhydrous toluene (20 mL), and added at 0° C. to a 20% phosgene solution in toluene (5.0 mL) diluted with anhydrous toluene (30 mL). The mixture was stirred at ambient temperature overnight.

The reaction solution was cautiously evaporated to dryness. The residue was precipitated from MTBE (15 mL). The obtained product was collected by filtration under an argon atmosphere, washed with MTBE (10 mL×2) and dried in vacuo for 6 hours. Yield: 0.9 g. 1H NMR (CDCl₃): δ (ppm) 4.66 (dq, —CH₂F, 2H), 3.93 (dt, —N(CO)—CH₂—CH₂F, 2H), 3.63 (bs, PEG backbone), 3.37 (s, —OCH₃, 3H).

3.3 Preparation of mPEG-N(CH₂CH₂F)— CO-5-(3,5-bis(trifluoromethyl)phenyl-2H-tetrazole, 5 kDa

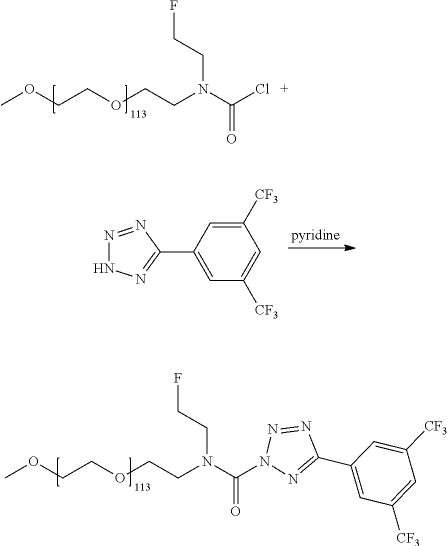

mPEG-N(CH₂CH₂F)—CO—Cl, 5 kDa (0.9 g) was added to a round bottom flask (100 mL), the flask was then capped with a septum and charged with argon, followed by addition of a solution of 5-[3,5-bis(trifluoromethyl)phenyl]-2H-tetrazole (Activator 42® Solution; Sigma-Aldrich) in acetonitrile (0.25M, 5 mL). While stirring, anhydrous pyridine (1 mL) was added via syringe. The solution was stirred at ambient temperature for 48 hours under an argon atmosphere. The solvent was evaporated to dryness under reduced pressure, and the residue was precipitated following addition of MTBE (15 mL). The powder was collected by filtration, washed with IPA (10 mL) and MTBE (10 mL×2), and dried in vacuo over a weekend. Yield: 920 mg. 1H NMR (CDCl₃): δ (ppm) 8.67 (s, Ar, 2H), 8.02 (s, Ar, 1H), 4.76 (t, —CH₂—F, 2H), 4.05 (t, —N—CH₂—CH₂F, 2H), 3.63 (bs, PEG backbone), 3.36 (s, 3H).

The above exemplary syntheses may be suitably carried out using PEG starting materials of a variety of molecular weights as described herein.

### Example 4

### SYNTHESIS OF MPEG-N(CH₂CHF₂)—CO-5-(3,5-BIS(TRIFLUOROMETHYL)PHENYL)-2H-TETRAZOLE, 5 KDA (REAGENT 4)

4.1 Preparation of mPEG-NH(CH₂CHF₂), 5 kDa

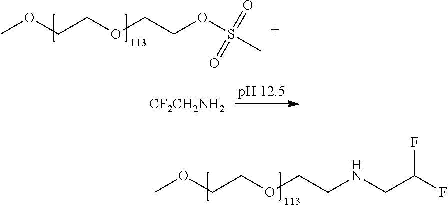

2,2-Difluoroethylamine (5.6 mL, 79 mmol) was dissolved in DI water (100 mL). The pH of the solution was adjusted to 12.5 by addition of 1N NaOH, followed by addition of mPEG-mesylate, 5 kDa (10 g, 2 mmol, prepared by the procedure described in Example 1.1) at room temperature. The mixture was stirred at 55-60° C. for 63 hours. NaCl was added in an amount sufficient to form 10% NaCl in the solution, followed by extraction with DCM (50 mL×3). The combined organic layers were dried over MgSO₄. After filtering off the solid, the filtrate was concentrated to dryness. The crude product was precipitated from MTBE (30 mL), filtered and washed with IPA (15 mL) followed by washing with MTBE containing 200 ppm BHT (10 mL×2). The obtained solid was dried in vacuo for 12 hours. Yield: 9.5 g.

The product was purified by cation exchange chromatography using a POROS 50HS column (Thermo Fisher). Yield: 5.1 g. Purity by HPLC: 100%. 1H-NMR (DMSO-d₆): δ (ppm) 5.97 (t, —CHF_{2, 1}H), 3.51 (bs, PEG backbone), 3.24 (s, —OCH₃, 3H), 2.89 (t, —N—CH₂—CHF_{2, 2}H), 2.70 (bs, —CH₂—NH—, 2H).

4.2 Preparation of mPEG-N(CH₂CHF₂)—CO—Cl, 5 kDa

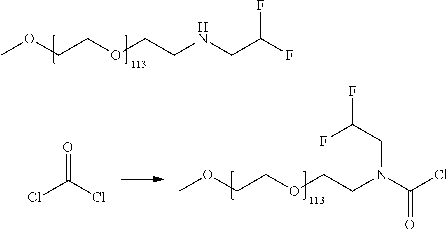

mPEG-NH(CH₂CHF₂), 5 kDa (3 g, 0.6 mmol) was dissolved in 30 mL of toluene in a round-bottom flask (100 mL). The solvent was then distilled off to dryness under reduced pressure, and the remaining residue was dissolved in a mixture of anhydrous DCM (15 mL) and anhydrous toluene (30 mL). The resulting solution was added at 0° C. to a 20% phosgene solution in toluene (9.0 mL) diluted with anhydrous toluene (30 mL). The mixture was stirred at ambient temperature overnight.

The solvents were cautiously evaporated to dryness. The residue was precipitated from MTBE (15 mL). The precipitated product was collected by filtration under argon, washed with MTBE (10 mL×2) and dried in vacuo overnight. Yield: 2.8 g. 1H NMR (CDCl₃): δ (ppm) 6.03 (tt, —CHF₂, ¹H), 3.95 (dt, —N—CH₂—CHF₂, 2H), 3.63 (bs, PEG backbone), 3.37 (s, —OCH₃, 3H).

4.3 Preparation of mPEG-N(CH₂CHF₂)— CO-5-(3,5-bis(trifluoromethyl)phenyl)-2H-tetrazole, 5 kDa

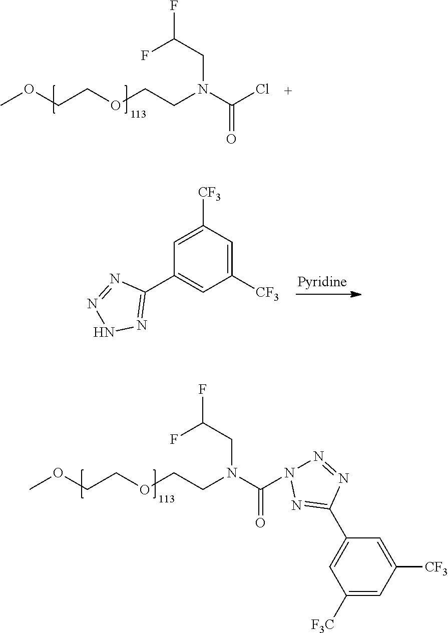

mPEG-N(CH₂CHF₂)—CO—C1 5 kDa (2.6 g) was added to a round bottom flask (100 mL); the flask was then capped with a septum and charged with argon. Next, a solution of 5-[3,5-bis(trifluoromethyl)phenyl]-2H-tetrazole in acetonitrile (0.25M, 10 mL; Activator 42® Solution; Sigma-Aldrich) was added. While stirring, anhydrous pyridine (1 mL) was added through a syringe. The solution was stirred for 48 hours at ambient temperature under an argon atmosphere, followed by removal of solvent by evaporation to dryness under reduced pressure. The residue was precipitated with MTBE (25 mL). The powder was collected by filtration, washed with IPA (15 mL) and MTBE (10 mL×2) and dried in vacuo overnight. Yield: 2.4 g 1H NMR (CDCl₃): δ (ppm) 8.69 (s, Ar, 2H), 8.03 (s, Ar, 1H), 6.25 (t, —CHF₂, 1H), 4.13 (t, —N—CH₂—CHF₂, 2H), 3.90 (t, —CH₂—N(CH₂CHF₂)—CO—, 2H), 3.63 (bs, PEG backbone), 3.36 (s, —OCH₃, 3H).

The above exemplary syntheses may be suitably carried out using PEG starting materials of a variety of molecular weights as described herein.

### Example 5

### SYNTHESIS OF MPEG-N(CH₂CF₃)—CO-5-(3,5-BIS(TRIFLUOROMETHYL)PHENYL)-2H-TETRAZOLE 5 KDA (REAGENT 5)

5.1 Preparation of mPEG-NH₂, 5 kDa

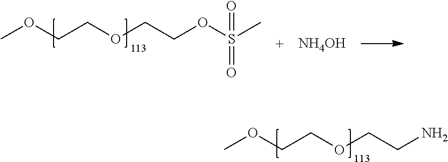

mPEG-mesylate 5 kDa (20 g, 4 mmol, prepared by the procedure described in Example 1.1) was stirred with ammonium hydroxide (500 mL) containing ammonium chloride (30 g) at 25° C. for 48 hours. NaCl (50 g) and DI water (200 mL) were added to the reaction mixture, followed by extraction with DCM (100 mL×4). The extracts were combined and treated with 1N HCl (0.5 mL), dried over sodium sulfate (15 g), and filtered. The filtrate was concentrated to dryness. The residue was precipitated from MTBE (100 mL). The crude product was collected by filtration, washed with IPA (25 mL×2), then with MTBE containing 200 ppm of BHT (25 mL×2), and dried in vacuo overnight. Yield: 19.0 g.

A portion of the crude product (15 g) was purified by cation exchange chromatography using POROS 50HS column. Yield 12.5 g. Purity by HPLC: 98.5%. Substitution by 1H-NMR: 93.0%. 1H NMR (DMSO-d₆): δ (ppm) 3.51 (bs, PEG backbone), 3.24 (s, —OCH₃, 3H), 2.65 (t, —CH₂—NH₂, 2H).

5.2 Preparation of mPEG-NH(COCF₃), 5 kDa

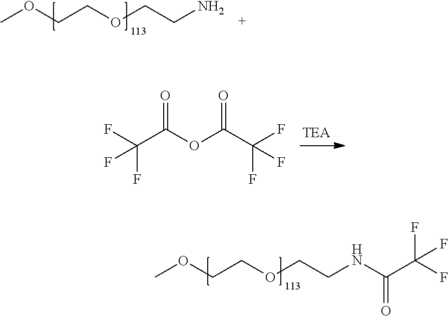

mPEG-amine, 5 kDa (6.0 g, 1.2 mmol) and triethylamine (1.8 mL, 10 eq) were mixed in anhydrous DCM (45 mL), followed by addition of trifluoroacetic anhydride (0.72 mL, 4.5 eq) while stirring. After stirring for 1 hour at room temperature, the solution was concentrated to dryness. The resulting residue was precipitated by addition of MTBE (30 mL). The solid was collected by filtration, washed with IPA (20 mL) and MTBE (20 mL×2). The obtained product was dried in vacuo for overnight. Yield: 6.0 g. Purity by HPLC: 100%. 1H-NMR (DMSO-d₆): δ (ppm) 3.51(bs, PEG backbone), 3.24 (s, —OCH₃, 3H).

5.3 Preparation of mPEG-NH(CH₂CF₃), 5 kDa

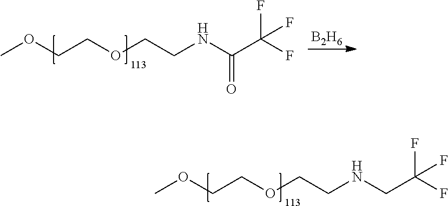

A solution of mPEG-NH(COCF₃), 5 kDa (6.0 g, 1.2 mmol) and BHT (10 mg) in anhydrous toluene (200 mL) was azeotropically distilled to dryness. Anhydrous THF (100 mL) was added to the flask and chilled in an ice bath, followed by dropwise addition of borane solution in THF (0.9 M, 30 mL, 27 mmol). After stirring for 2 hours at 0° C., the mixture was stirred under reflux under an argon atmosphere for 72 hours.

Next the reaction mixture was cooled to room temperature, methanol (100 mL) was added and the mixture was stirred under reflux for another 5 hours. Following evaporation of the solvent, the residue was dissolved in a 10% NaCl aqueous solution. The pH of the solution was adjusted with 1N NaOH to 11. The crude product was extracted with DCM three times (50 mL ×3). The organic layers were combined and dried over MgSO₄. After the solid impurities were filtered off, the filtrate was evaporated to dryness and the crude product was precipitated with MTBE (50 mL). The solid precipitate was collected by filtration, washed with MTBE (100 mL) containing BHT (30 mg), and dried in vacuo overnight. Yield: 5.7 g.

Next the product was dissolved in 1N NaOH (30 mL) and the solution was stirred at room temperature overnight, and was isolated by the above described workup procedure. Finally, the product was purified by cation exchange chromatography using POROS 50HS column. Yield: 1.5 g. Purity by HPLC: 100%. 1H-NMR (DMSO-d₆): δ (ppm) 3.51(bs, PEG backbone), 3.24 (m, —OCH₃ & —N—CH₂—CF₃, 5H), 2.75 (q, —CH₂—NH—, 2H), 2.25 (m, —NH—).

5.4. Preparation of mPEG-N(CH₂CF₃)—CO—C₁, 5 kDa

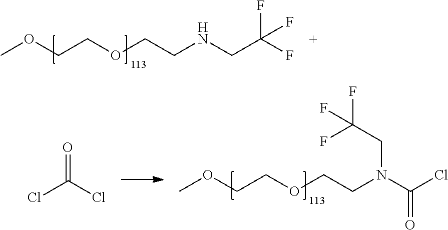

mPEG-NH(CH₂CF₃) 5 kDa (0.9 g, 0.18 mmol) was dissolved in 30 mL of toluene in a round-bottom flask (100 mL). Next, the solvent was distilled off to dryness under reduced pressure. The residue was dissolved in a mixture of anhydrous DCM (15 mL) and anhydrous toluene (20 mL). The obtained solution was added at 0° C. to a 20% phosgene solution in toluene (5.0 mL) diluted with anhydrous toluene (30 mL). The mixture was stirred at ambient temperature overnight.

The reaction solution was cautiously concentrated to dryness. The residue was precipitated from MTBE (50 mL). The product was collected by filtration under argon, washed twice with MTBE (5 mL×2) and dried in vacuo for 3 hours. Yield: 0.84 g. 1H NMR (CDCl₃): δ (ppm) 4.34 (q, —N—CH₂—CF₃, 1H), 4.21 (q, —N—CH₂—CF₃, 1H), 3.63 (bs, PEG backbone), 3.37 (s, —OCH₃, 3H).

5.5 Preparation of mPEG-N(CH₂CF₃)—CO-5-(3,5-bis(trifluoromethyl)phenyl)-2H-tetrazole, 5 kDa

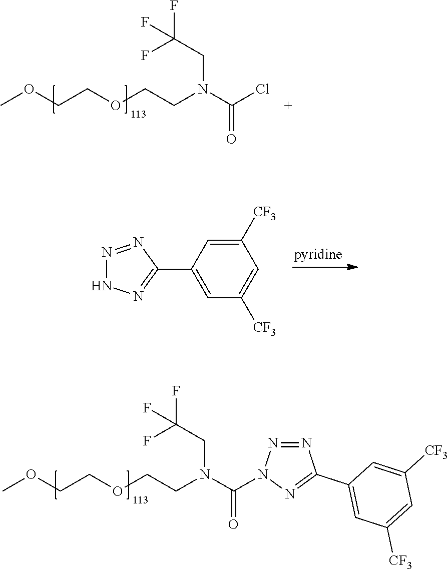

mPEG-N(CH₂CF₃)—CO—Cl, 5 kDa (0.84 g) was added in a round bottom flask (25 mL). The flask was then capped with a septum and charged with argon, followed by addition of 5-[3,5-bis(trifluoromethyl)phenyl]-2H-tetrazole solution in acetonitrile (0.25M, 5 mL; Activator 42® Solution; Sigma-Aldrich). While stirring, anhydrous pyridine (1 mL) was added through a syringe. The solution was stirred at ambient temperature for 48 h under an argon atmosphere. The solvent was then evaporated to dryness under reduced pressure, and the residue was precipitated by addition of MTBE (15 mL). The product was collected by filtration, washed with IPA (10 mL) followed by washing with MTBE (10 mL×2) and dried in vacuo for 5 hours. Yield: 0.80 mg. 1H NMR (CDCl₃): δ (ppm) 8.70 (s, Ar, 2H), 8.03 (s, Ar, 1H), 4.68 (br, —N—CH₂—CF₃, 2H), 3.96 (s, —CH₂—N—CH₂CF₃, 2H), 3.63 (bs, PEG backbone), 3.37 (s, —OCH₃, 3H).

The above exemplary syntheses may be suitably carried out using PEG starting materials of a variety of molecular weights as described herein.

### Example 6

### SYNTHESIS OF MPEG-N(CH₂C6F₅)—CO-5-(3,5-BIS(TRIFLUOROMETHYL)PHENYL)-2H-TETRAZOLE 5 KDA (REAGENT 6)

6.1. Preparation of mPEG-NH(CH₂C6F₅) 5 kDa

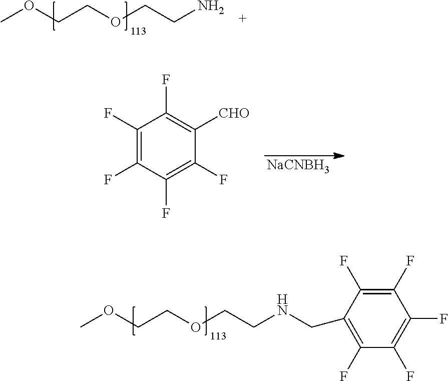

mPEG-amine, 5 kDa (3 g, 0.6 mmol, prepared by the procedure described in Example 5.1), pentafluorobenzaldehyde (700 μL, 10 eq) and sodium cyanoborohydride (150 mg, 4.2 eq) were mixed in DMF (20 mL). The mixture was stirred at ambient temperature overnight. After evaporation of solvent at reduced pressure at 30° C., the residue was precipitated with MTBE (20 mL). The solid was collected by filtration and sequentially washed with IPA (15 mL) and then with MTBE (10 mL×2). The filter cake was dried in vacuo for 2 hours. Yield: 3.1 g. Purity by HPLC: 90%.

The crude product was purified by cation exchange chromatography using POROS 50HS column. Yield: 1.4 g. Purity by HPLC: 96.5%; Substitution by 1H-NMR: ˜100%. 1H-NMR (DMSO-d₆): δ (ppm) 3.82 (s, —N—CH₂—C6F₅, 2H), 3.51(bs, PEG backbone), 3.24 (s, —OCH₃, 3H), 2.62 (t, —CH₂—N—, 2H).

6.2. Preparation of mPEG-N(CH₂C6F₅)—CO—C₁, 5 kDa

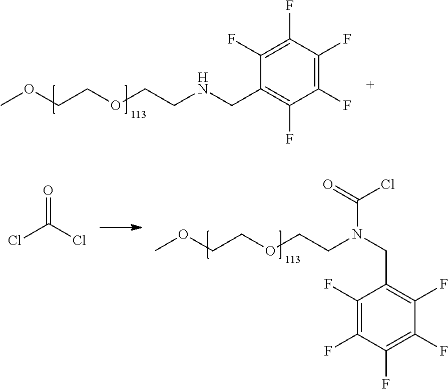

mPEG-NH(CH₂C6F₅), 5 kDa (1.0 g, 0.2 mmol) was dissolved in 30 mL of toluene in a round-bottom flask (100 mL), followed by removal of solvent by distillation to dryness under reduced pressure. The residue was dissolved in a mixture of anhydrous DCM (15 mL) and anhydrous toluene (30 mL). The obtained solution was added at 0° C. to a 20% phosgene solution in toluene (9.0 mL) diluted with anhydrous toluene (30 mL), and the mixture stirred at ambient temperature overnight.

The solvents were cautiously evaporated to dryness. The residue was then precipitated by addition of MTBE (15 mL). The powder was collected by filtration under argon, washed twice with MTBE twice (10 mL×2) and dried in vacuo for 6 hours. Yield: 1.0 g. 1H NMR (CDCl₃): δ (ppm) 4.93 (s, —N—CH₂—C6F₅, 1H), 4.76 (s, —N—CH₂—C6F₅, 1H), 3.63 (bs, PEG backbone & —CH₂—N—COCl), 3.37 (s, —OCH₃, 3H).

6.3. Preparation of mPEG-N(CH₂C6F₅)—CO-5-(3,5-bis(trifluoromethyl)phenyl)-2H-tetrazole, 5 kDa

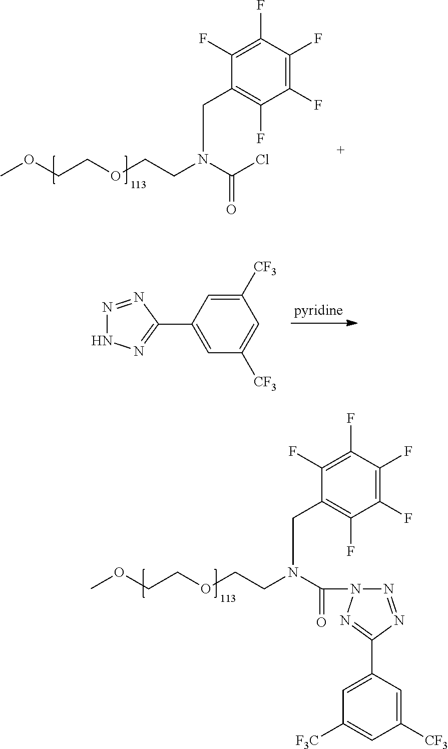

mPEG-N(CH₂C6F₅)—CO—C₁, 5 kDa (1.0 g) was added to a round bottom flask (100 mL), the flask was then capped with a septum and charged with argon. Next a solution of 5-[3,5-bis(trifluoromethyl)phenyl]-2H-tetrazole (Activator 42® Solution; Sigma-Aldrich) in acetonitrile (0.25M, 5 mL) was added. Then, while stirring, anhydrous pyridine (1 mL) was added through a syringe. The solution was stirred under argon at ambient temperature for 48 h. Next the solvent was evaporated to dryness under reduced pressure, and the residue was precipitated by addition of MTBE (15 mL). The product was collected by filtration, washed with IPA three times (10 mL×3) and MTBE twice (10 mL×2), and dried in vacuo for overnight. Yield: 0.85 mg. 1H NMR (CDCl₃): δ (ppm) 8.78 (s, Ar, 2H), 8.02 (s, Ar, 1H), 5.04 (bs, —N(CO)—CH₂—C6F₅, 2H), 3.63 (bs, PEG backbone), 3.37 (s, —OCH₃, 3H).

The above exemplary syntheses may be suitably carried out using PEG starting materials of a variety of molecular weights as described herein.

### Example 7

### SYNTHESIS OF MPEG-OCO—NH—(PIPERIDIN-4-YL)-CO-5-(3,5-BIS(TRIFLUOROMETHYL)PHENYL)-2H-TETRAZOLE 5 KDA (REAGENT 7)

7.1. Preparation of mPEG-OCO—NH-(piperidin-4-yl) 5 kDa

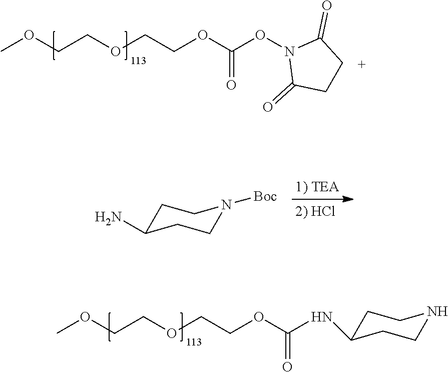

Step 1. 4-amino-1-Boc-piperidine (2.2 g, 11 mmol) and BHT (20 mg) were dissolved in DCM (50 mL), followed by addition of mPEG-SC 5 kDa (5.5 g, 1.1 mmol, prepared by the procedure described in Example 2.1) and TEA (4 mL, 29 mmol). The mixture was stirred at ambient temperature overnight under an argon atmosphere. The solution was concentrated to dryness and the product was precipitated with MTBE (50 mL). The solid was collected by filtration, washed with IPA once (25 mL), then with MTBE twice (25 mL×2). The solid was dried in vacuo for 1 hour. Yield: 5.4 g.

Step 2. The resulting solid and BHT (50 mg) were dissolved in dioxane (10 mL) and chilled in an ice bath. HCl in dioxane (4M, 15 mL) was slowly added to the suspension while stirring, and the mixture was stirred at 0° C. to ambient temperature overnight. The solution was concentrated to dryness. The residue was dissolved in DI water (200 mL), and the pH of the solution was adjusted to pH 10.5-11 with Na₂CO₃ (20 g). After saturation to 10% NaCl by addition of sodium chloride, the solution was extracted with DCM (40 mL×3). The combined extracts were dried over MgSO₄. After filtering off the solid, the filtrate was evaporated to dryness. The crude product was purified by cation exchange chromatography using POROS 50HS column. Yield: 3.5 g; Purity by HPLC: 98.3%. 1H-NMR (DMSO-d₆): δ (ppm) 4.03 (t, —CH₂—OCO—, 2H), 3.51 (bs, PEG backbone & —N—CH<), 3.24 (s, —OCH₃, 3H), 2.88 (dd, NH(CH₂—)—CH₂—, 2H), 2.44 (dd, NH (CH₂—)—CH₂—, 2H), 1.64 (dd, —NHCH(CH₂—)—CH₂—, 2H), 1.29 (m, —NHCH(CH₂—)—CH₂—, 2H).

7.2. Preparation of mPEG-OCO—NH-(1-COCl-piperidin-4-yl), 5 kDa

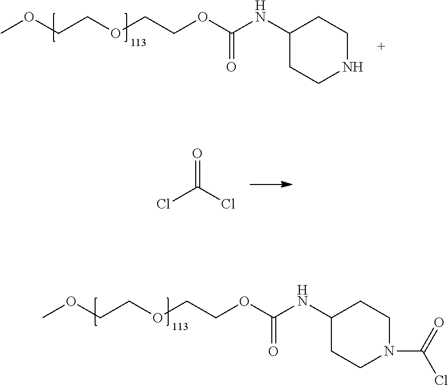

mPEG-OCO—NH-(piperidin-4-yl), 5 kDa (610 mg, 0.12 mmol) was dissolved in 50 mL of toluene in a round-bottom flask (100 mL). Next, the solvent was distilled off to dryness under reduced pressure. The residue was dissolved in a mixture of anhydrous DCM (15 mL) and anhydrous toluene (30 mL). The obtained solution was added at 0° C. to a 20% phosgene solution in toluene (5.0 mL) diluted with anhydrous toluene (30 mL). The mixture was stirred at ambient temperature overnight.

The solvent was cautiously evaporated to dryness. The residue was precipitated with MTBE (30 mL). The product was collected by filtration under argon, washed with MTBE twice (15 mL×2) and dried in vacuo for 3 hours. Yield: 540 mg. 1H NMR (CDCl₃): δ (ppm) 4.21-4.24 (br, —CH₂—OCO— & —CH₂(—CH₂)N—COCl, 4H), 3.63 (bs, PEG backbone & —OCONH—CH<), 3.36 (s, —OCH₃, 3H), 3.2-3.0 (dt, —CH₂(—CH₂)N—COCl, 2H), 2.00 (br, —CH(CH₂—)CH₂—, 2H), 1.44 (br, —CH(CH₂—)CH₂—, 2H).

7.3. Preparation of mPEG-OCO—NH-(piperidin-4-yl)-CO-5-(3,5-bis(trifluoromethyl)phenyl)-2H-tetrazole, 5 kDa

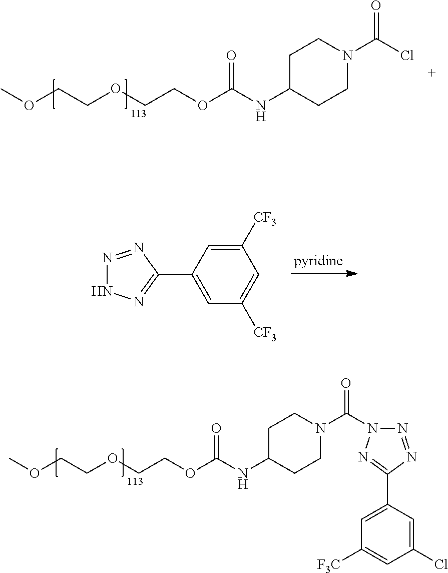

mPEG-OCO—NH-(1-COCl-piperidin-4-yl), 5 kDa (0.54 g) was added to a round bottom flask (25 mL), and the flask was capped with a septum and charged with argon. Next 5-[3,5-bis(trifluoromethyl)phenyl]-2H-tetrazole solution in acetonitrile (0.25M, 5 mL; Activator 42® Solution; Sigma-Aldrich) was added. While stirring, anhydrous pyridine (1 mL) was added via syringe. The solution was stirred under argon at ambient temperature for 24 h. The solvent was evaporated to dryness under reduced pressure, and the residue was precipitated with MTBE (40 mL). The product was collected by filtration, washed with IPA once (30 mL), and then washed twice with MTBE (10 mL×2) and dried in vacuo overnight. Yield: 0.49 g. 1H NMR (DMSO-d₆): δ (ppm) 8.65 (s, Ar, 2H), 8.43 (s, Ar, 1H), 7.44 (s, —NH—, 1H), 4.21 (d, —NH—CH<, 1H), 4.06 (s, —CH₂—OCO—,2H), 3.51 (bs, PEG backbone & —(CH₂)₂—N—CO—), 3.24 (s, —OCH₃, 3H), 1.99-1.80 (dd, CH(CH₂—)CH₂—,2H), 1.53 (m, CH(CH₂—)CH₂—,2H).

The above exemplary syntheses may be suitably carried out using PEG starting materials of a variety of molecular weights as described herein.

### Example 8

### SYNTHESIS OF MPEG-OCO—NH—CH₂—(PIPERIDIN-4-YL)-CO-5-(3,5-BIS(TRIFLUOROMETHYL)PHENYL)-2H-TETRAZOLE 5 KDA (REAGENT 8)

8.1. Preparation of mPEG-OCO—NH—CH₂-(piperidin-4-yl), 5 kDa

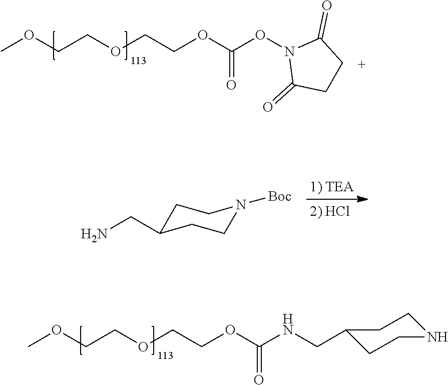

4-aminomethyl-1-Boc-piperidine (2.2 g, 11 mmol) and BHT (20 mg) were dissolved in DCM (50 mL), followed by addition of mPEG-SC 5 kDa (5.0 g, 1 mmol, prepared by the procedure described in Example 2.1) and TEA (4 mL, 29 mmol). The mixture was stirred at ambient temperature overnight under an argon atmosphere. Next the solvent was removed by distillation to dryness. The crude product was precipitated with MTBE (50 mL), filtered off and washed with IPA (25 mL) and MTBE (25 mL×2), then dried in vacuo for 2 hours. Yield: 4.77 g.

The crude product and BHT (20 mg) were dissolved in dioxane (5 mL), followed by addition of 4M HCl in dioxane (20 mL) while chilled in an ice bath. The solution was allowed warm with stirring from 0° ° C. to ambient temperature overnight. After evaporation of solvent, the residue was precipitated from MTBE (50 mL). Following filtration, the filter cake was dissolved in 100 mL of DI water. The pH of the solution was adjusted to pH 11 using Na₂CO₃. After saturating with NaCl to 10% NaCl, the solution was extracted with DCM three times (40 mL×3). The combined extracts were dried over MgSO₄, filtered, and the filtrate was evaporated to dryness. The residue was precipitated with MTBE (50 mL). The crude product was collected by filtration and dried in vacuo for 2 hours. Weight: 4.6 g.

The crude product was purified by cation exchange chromatography using s POROS 50HS column. Weight: 3.3 g. Purity by HPLC: 98.8%. 1H-NMR (DMSO-d₆): δ (ppm) 4.03 (t, —CH₂—OCO—, 2H), 3.51 (bs, PEG backbone), 3.24 (s, —OCH₃, 3H), 2.90 (dt, —CONH—CH₂—, 2H), 2.82 (t, —CH₂—NH(CH₂—), 2H), 2.37 (dt, —CH₂—NH(CH₂—)—, 2H), 1.55 (bd, —CH(CH₂—)CH₂—, 2H), 1.45 (br, —CH₂—CH<, 1H), 0.97 (dq, —CH(CH₂—)—CH₂—, 2H).

8.2. Preparation of mPEG-OCO—NH—CH₂-(1-COCl-piperidin-4-yl), 5 kDa

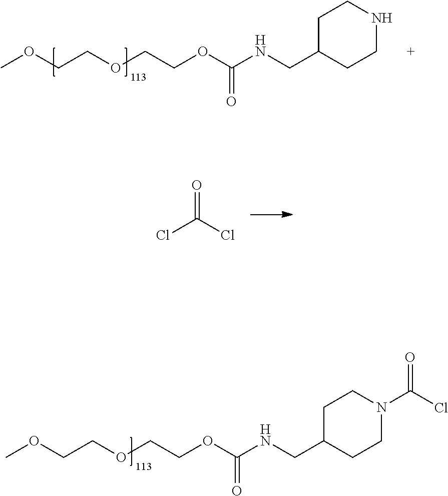

mPEG-OCO—NH—CH₂-(piperidin-4-yl), 5 kDa (500 mg, 0.12 mmol) was dissolved in 50 mL of toluene in a round-bottom flask (100 mL). Next the solvent was removed by distillation to dryness under reduced pressure. The residue was dissolved in a mixture of anhydrous DCM (15 mL) and anhydrous toluene (30 mL), and the resulting solution was added at 0° C. to a solution of 20% phosgene in toluene (5.0 mL) diluted with anhydrous toluene (30 mL). The mixture was stirred at ambient temperature for overnight.

Next the solvent was cautiously evaporated to dryness. The residue was precipitated from MTBE (50 mL). The product was collected by filtration under argon, washed twice with MTBE (15 mL×2), and dried in vacuo for 3 hours. Yield: 400 mg. 1H NMR (CDCl₃): δ (ppm) 5.00 (t, —OCONH—, 1H), 4.34 (d, —OCONH—CH₂—, 2H), 4.20 (t, —CH₂—OCO—, 2H), 3.63 (bs, PEG backbone), 3.37 (s, —OCH₃, 3H), 3.08-2.86 (m, —CH₂—N(COCl)—CH₂-4H), 1.77 (br, —CH(CH₂—)CH₂—, 3H), 1.24 (br, —CH(CH₂—)—CH₂—, 2H).

8.3. Preparation of mPEG-OCO—NH—CH₂-(piperidin-4-yl)-CO-5-(3,5-bis(trifluoromethyl) phenyl)-2H-tetrazole, 5 kDa

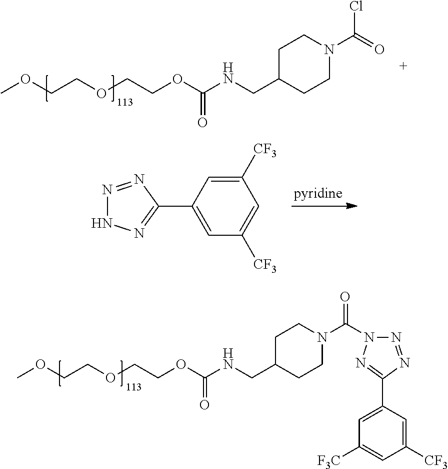

mPEG-OCO—NH—CH₂-(1-COCl-piperidin-4-yl), 5 kDa (0.40 g) was added to a round bottom flask (25 mL), the flask was then capped with a septum and charged with argon, followed by addition of 5-[3,5-bis(trifluoromethyl)phenyl]-2H-tetrazole solution in acetonitrile (0.25M, 5 mL; Activator 42® Solution; Sigma-Aldrich). While stirring, anhydrous pyridine (1 mL) was added through a syringe. The solution was stirred for 72 hours at ambient temperature under an argon atmosphere. Next the solvent was removed by distillation to dryness under reduced pressure. The residue was precipitated from MTBE (40 mL), collected by filtration, washed with IPA once (30 mL) for 60 minutes and MTBE twice (15 mL×2), and dried in vacuo for 3 hours. Yield: 0.38 g. 1H NMR (CDCl₃): δ (ppm) 8.69 (s, Ar, 2H), 8.02 (s, Ar, 1H), 5.06 (t, —CONH—, 1H), 4.55 (d, —CONH—CH₂—, 1H), 4.21 (t, —CH₂—OCO—, 2H), 3.63 (bs, PEG backbone), 3.37 (s, —OCH₃, 3H), 3.20 (br, —CH₂—N(CH₂—)—CO—, 4H), 2.01 (br, —CH₂—CH(CH₂—)CH₂—, 1H), 1.84 (br, —CH(CH₂—)CH₂—, 2H), 1.42 (t, —CH(CH₂—)CH₂—, 2H).

The above exemplary syntheses may be suitably carried out using PEG starting materials of a variety of molecular weights as described herein.

### Example 9

### SYNTHESIS OF MPEG-OCO-(2-CF₃-PIPERAZIN-1-YL)-CO-5-(3,5-BIS(TRIFLUOROMETHYL)PHENYL)-2H-TETRAZOLE, 5 KDA (REAGENT 9)

9.1. Preparation of mPEG-OCO—Cl, 5 kDa

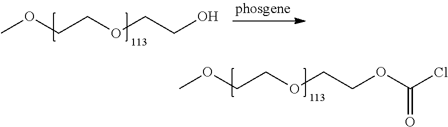

mPEG-OH 5 kDa (4.0 g, 0.8 mmol) was dissolved in anhydrous toluene (100 mL). Next the solvent was distilled off to dryness. The residue was dissolved in a mixture of anhydrous toluene (20 mL) and anhydrous DCM (10 mL). While the solution was stirred at argon atmosphere, 20%-phosgene solution in toluene (2 mL, 5 eq) was added and the mixture was stirred overnight under argon at the ambient temperature for. The solvents were cautiously evaporated to dryness, then the crude product was precipitated with MTBE (100 mL). The precipitate was collected by filtration and washed with MTBE (50 mL×2). The product was dried in vacuo for 3 h. Yield: 4.0 g. NMR showed 92.3% substitution. 1H-NMR (CDCl₃): δ (ppm) 4.44 (t, —CH₂—OCO—C1, 2H), 3.63 (bs, PEG backbone), 3.37 (s, —OCH₃, 3H).

9.2. Preparation of mPEG-OCO-(2-CF₃-piperazin-1-yl) 5 kDa

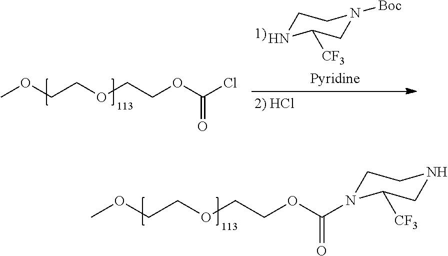

mPEG-OCO—C1 5 kDa was dissolved in anhydrous DCM (25 mL). While stirring under argon, 4-Boc-2-trifluoromethyl-piperazine (250 mg, 1.3 eq) and anhydrous pyridine (3 mL) were sequentially added. The reaction mixture was stirred overnight at ambient temperature under an argon atmosphere. Following concentration, the residue was precipitated from MTBE (50 mL). The solid was collected by filtration, washed with IPA twice (25 mL×2) for 2 hours. The powder was collected again by filtration. The filter cake was washed with MTBE (25 mL×2) and dried in vacuo overnight. Yield: 3.8 g.

The resulting powder and BHT (20 mg) were dissolved in dioxane (10 mL), followed by addition of 4M HCl in dioxane (15 mL) while chilled in an ice bath. The solution was warmed with stirring from 0° ° C. to ambient temperature overnight. After evaporation of the solvent, the residue was precipitated with MTBE (50 mL). The precipitate was collected by filtration and then dissolved in 100 mL of DI water. The pH of the solution was adjusted to 11 using Na₂CO₃. After saturating the solution to 10% NaCl, the product was recovered by extraction with DCM (40 mL×3). The combined extracts were dried over MgSO₄, filtered, and the filtrate was evaporated to dryness. The residue was treated with MTBE (50 mL). The solid was collected by filtration, dried in vacuo for 2 hours. Yield: 3.7 g. Purity by HPLC: 25.8%.

The residue was then purified by cation exchange chromatography using POROS 50HS column. Yield: 720 mg. Purity by HPLC: 100%. 1H-NMR (DMSO-d₆): δ (ppm) 4.54 (br, CF₃—CH—, 1H), 4.16 (t, —CH₂—OCO—, 2H), 3.82 (d, —CO—N(CH—)—CH₂—, 1H), 3.51 (bs, PEG backbone), 3.24 (s, —OCH₃, 3H), 3.19 (d, —CO—N(CH—)—CH₂—, 1H), 2.90-2.80 (br, —CH₂—NH—CH₂—, 3H), 2.54 (t, —CH₂—NH—CH₂—, 1H).

9.3. Preparation of mPEG-OCO-(4-COC1-2-CF₃-piperazin-1-yl), 5 kDa

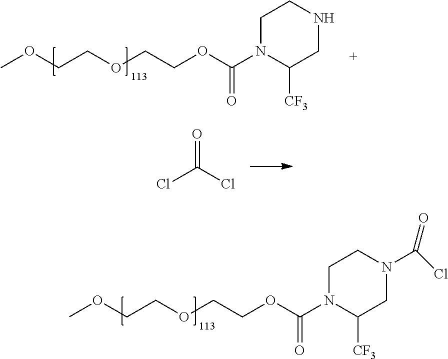

mPEG-OCO-(2-CF₃-piperazin-1-yl), 5 kDa (310 mg, 0.06 mmol) was dissolved in 15 mL of toluene in a round-bottom flask (100 mL). Then the solvent was distilled off to dryness under reduced pressure. Next the residue was dissolved in a mixture of anhydrous DCM (10 mL) and anhydrous toluene (30 mL). The obtained solution was added at 0° C. to 20% phosgene solution in toluene (5.0 mL) diluted with anhydrous toluene (5 mL). The mixture was stirred at ambient temperature for overnight.

The reaction solution was cautiously evaporated to dryness. The residue was precipitated from MTBE (20 mL). The product was collected by filtration under argon, washed with MTBE (10 mL×2) and dried in vacuo for 4 hours. Yield: 230 mg. 1H NMR (CDCl₃): δ (ppm) 4.81 (br, CF₃—CH—, 1H), 4.64 (br, —CH₂—N(COCl)—, 1H), 4.32 (br, —CH₂—OCO—N<& —CH₂—N(COCl)—, 4H), 4.15 (br, >N—CH₂—, 1H), 3.63 (bs, PEG backbone), 3.37 (s, —OCH₃, 3H), 3.26-3.02 (m, >N—CH₂—CH₂—N(COCl)—, 3H).

9.4. Preparation of mPEG-OCO-(1-(2-CF₃-piperazinyl)-4-CO-5-(3,5-bis(trifluoromethyl) phenyl)-2H-tetrazole), 5 kDa

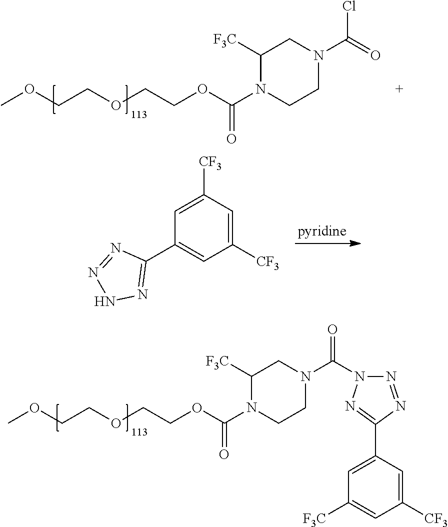

mPEG-(4-chlorocarbonyl-2-trifluoromethyl-piperazine-1-yl)-carboxylate 5 kDa (0.23 g) was added to a round bottom flask (25 mL). The flask was then capped with a septum and charged with argon, followed by addition of the 5-[3,5-bis(trifluoromethyl)phenyl]-2H-tetrazole solution in acetonitrile (0.25M, 5 mL; Activator 42® Solution; Sigma-Aldrich). While stirring, anhydrous pyridine (0.5 mL) was added through a syringe. The solution was stirred under argon atmosphere at ambient temperature for overnight. The solvent was evaporated to dryness under the reduced pressure. The product was precipitated with MTBE (40 mL). The precipitate was collected by filtration, washed with IPA once (30 mL) for 30 min and MTBE twice (10 mL×2), and dried in vacuo for overnight. Yield: 0.17 g. 1H NMR (DMSO-d₆): δ(ppm) 8.67 (s, Ar, 2H), 8.45 (s, Ar, 1H), 4.95 (br, CF₃—CH—, 1H), 4.22-3.96 (br, —CH₂—OCO— & CF₃CH—CH₂—N(CO—)—, 4H), 3.51 (bs, PEG backbone), 3.24 (s, —OCH₃, 3H).

The above exemplary syntheses may be suitably carried out using PEG starting materials of a variety of molecular weights as described herein.

### Example 10

### REACTIVITY OF REAGENTS 1-9 WITH α-CBZ-HISTIDINE AT PH 5.0, 5.5, 6.0, AND 6.5 AT 25° C. (1:10)—AN INVESTIGATION OF HISTIDINE-REACTIVITY

Illustrative Reaction with Reagent 1:

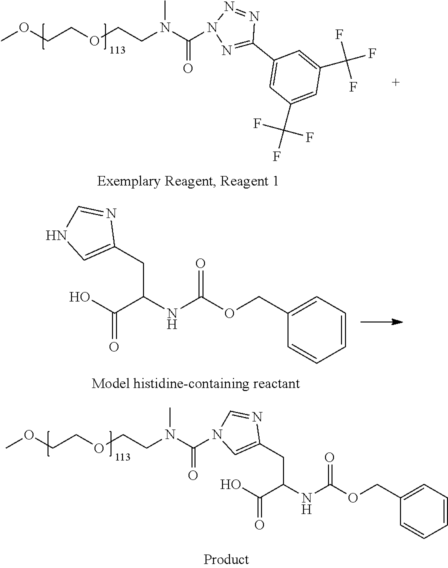

α-CBZ-histidine (5.79 mg, 0.020 mmol), as an illustrative histidine-containing model compound, was dissolved in 1.0 mL of 100 mM phosphate buffer at five different pHs (5.0, 5.5, 6.0, and 6.5). Reagent 1 (mPEG-N(CH₃)—CO-5-(3,5-bis(trifluoromethyl)phenyl)-2H-tetrazole) (10 mg, 0.002 mmol) was added to each solution. The resulting mixtures were stirred at 25° C. and analyzed by RP-HPLC at different time intervals.

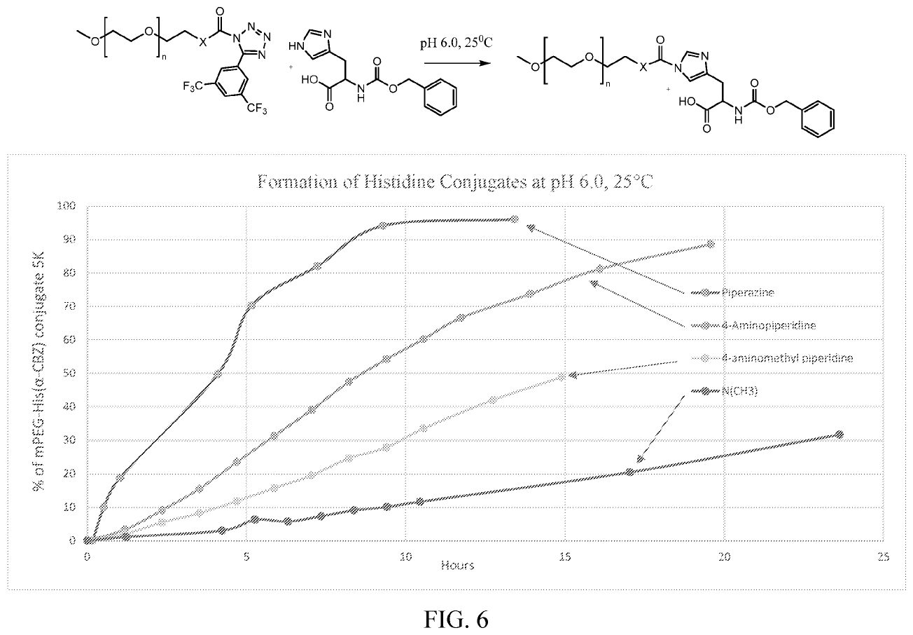

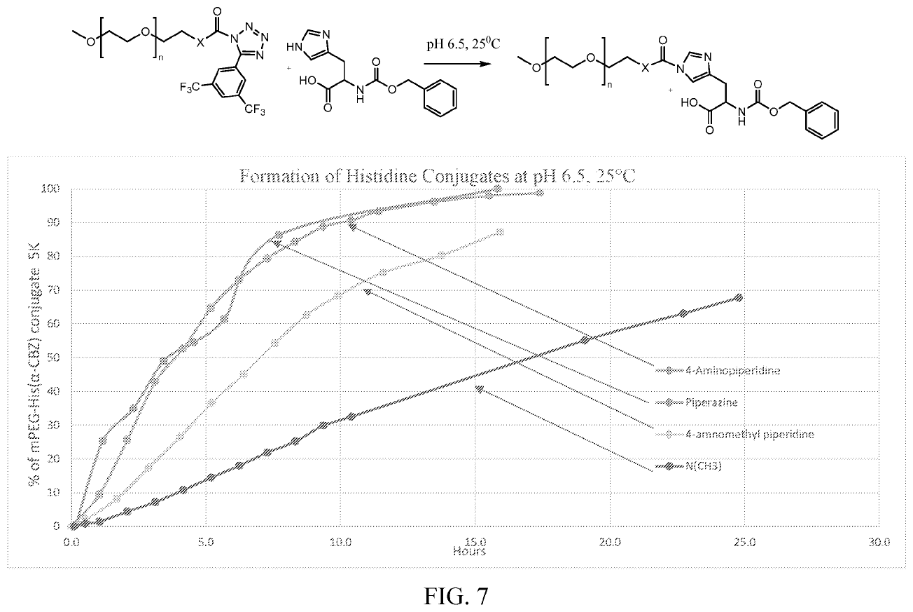

Reactivity evaluations of Reagents 2 to 9 were carried out with α-CBZ-histidine at the pH ranges in the preceding paragraph at 25° ° C. The results are summarized in Table 1 (FIG. 2) and illustrated in FIG. 3 (pH 5.0), FIG. 5 (pH 5.5, 4 conjugates), FIG. 6 (pH 6.0, 4 conjugates), FIG. 7 (pH 6.5, 4 conjugates), FIG. 8 (reactivity of Reagent 7), and FIG. 9 (reactivity of Reagent 8).

TABLE 1
Reactivity of PEG Reagents 1-9 with α-CBZ-Histidine, Rgt:His Molar Ratio 1:10,
phosphate buffer, 25° C.

| | | % of conjugate with CBZ-His after 5h | | | |
|---|---|---|---|---|---|
| Rgt | | at pH | | | |
| Name | Structure of PEG Reagent | 5.0 | 5.5 | 6.0 | 6.5 |
| Reagent 1 | 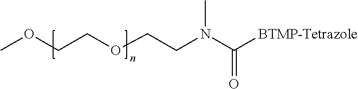 | <1 | < 1.5 | 6.0 | 14 |
| Reagent 2 | 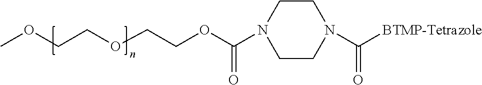 | 14 | 30 | 70 | 57 |
| Reagent 3 | 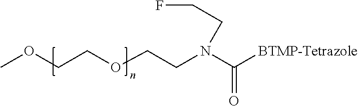 | <1 | | | |
| Reagent 4 | 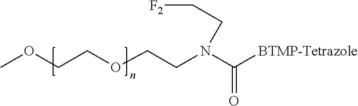 | 5 | | | |
| Reagent 5 | 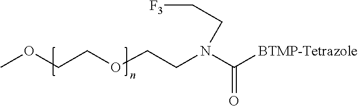 | 20 | | | |
| Reagent 6 | 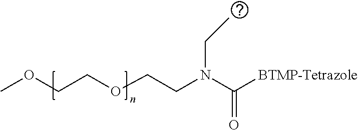 | 2 | | | |
| Reagent 9 | 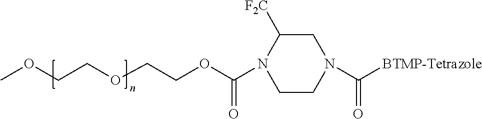 | 23 | 63 | | |
| Reagent 7 | 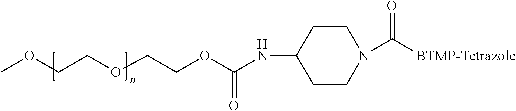 | 2 | 5 | 25 | 63 |
| Reagent 8 | 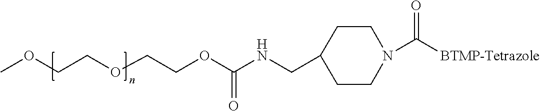 | 1 | 3 | 13 | 36 |

As shown in the table above, exemplary reagents in accordance with the disclosure having different overall spacer moieties intervening between the polymer moiety and the illustrative BTMP-tetrazole (or other suitable) leaving group (i.e., ˜(X)_{0.1}—(R₂)_{0.1}—NR₁—C(O)-BTMP-tetrazole) possess different reactivities with a histidine-containing reactant when evaluated under a series of pH conditions. These data support the feature of the polymer reagents described herein as being histidine-reactive, and further illustrate that the reactivity of the reagents can be effectively tailored by the selection of the components of the spacer moiety to achieve optimal reactivity suited for a given application and reaction condition. As can be seen, the reactivities of the reagents appear to generally follow a trend of increasing with increasing pH under the given conditions.

### Example 11

### CONJUGATION OF REAGENT 2 WITH α-CBZ-LYSINE AT PH 5.0 AND 5.5 AT 25° C. (1:10)—AN INVESTIGATION OF LYSINE-REACTIVITY

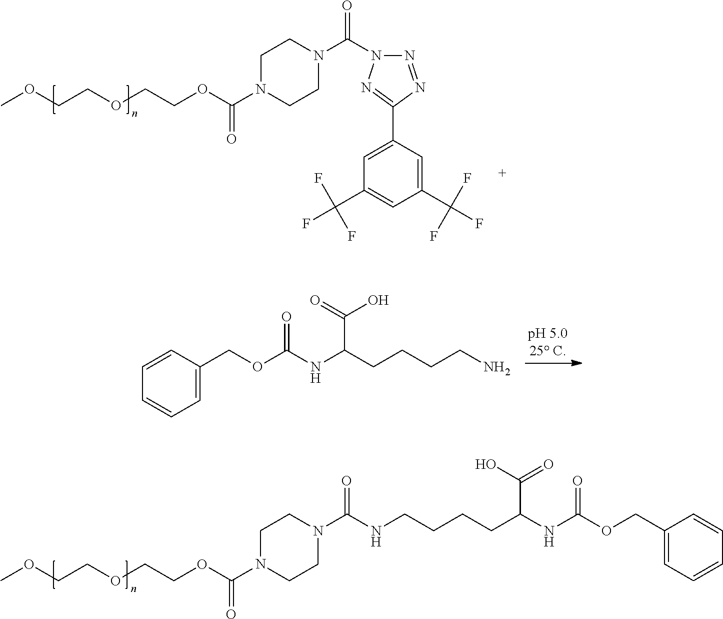

α-CBZ-Lysine (5.61 mg, 0.020 mmol), as an illustrative lysine-containing reactant, was dissolved in 1.0 mL of each of 100 mM phosphate buffer at pH 5.0 and 5.5, respectively. Reagent 2 (mPEG-Piperazine-CO-5-(3,5-bis(trifluoromethyl)phenyl)-2H-tetrazole)) (10 mg, 0.002 mmol) was added to each solution. The obtained solutions were stirred at 25° C. and analyzed by RP-HPLC at different time intervals. Results are provided in the table below.

Table 2. Reactivity of PEG Reagent 2 with Different Amino Acid or Oligopeptide Targets: α-CBZ-His, α-CBZ-Lys, and ω-CBZ-Lys-Gly-Gly-OH; PEG Reagent: Amino Acid or Tripeptide Molar Ratio 1:10, Phosphate Buffer, 25° C.

TABLE 2
Reactivity of PEG Reagent 2 with α-CBZ-His, α-CBZ-Lys, and ω-CBZ-Lys-
Gly-Gly-OH.
PEG-Reagent: aminoacid or tripeptide mol ratio 1:10, phosphate buffer, 25° C.

| | | | % of conjugate after 5 h | |
|---|---|---|---|---|
| Amino- | | Chemical Name | reaction | |
| acid | | of Exemplary | at pH | |
| Name | Structure of PEG Reagent | PEG Reagent | 5.0 | 5.5 |
| α-CBZ- His | 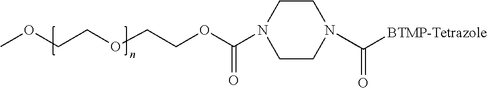 | mPEG- Piperazine-CO-5- (3,5- bis(trifluoromethyl) phenyl)- 2H-tetrazole, 5 kDa | 14 | 30 |
| α-CBZ- Lys | 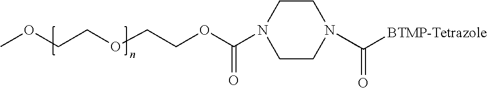 | mPEG- Piperazine-CO-5- (3,5- bis(trifluoromethyl) phenyl)- 2H-tetrazole, 5kDa | 0 | 0.5 |
| α-CBZ- Lys- Gly- Gly-OH | 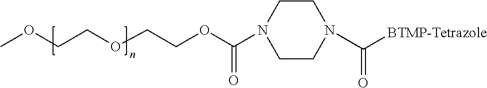 | mPEG- Piperazine-CO-5- (3,5- bis(trifluoromethyl) phenyl)- 2H-tetrazole, 5 kDa | 1.2 | 3.1 |

As can be seen from the results in Table 2, under the reaction conditions shown, exemplary Reagent 2 exhibits a significantly higher selectivity for reaction with the amino acid, histidine, when compared to reaction with the amino acid, lysine, as evidenced by reactions carried out with the model compounds, α-CBZ-histidine, α-CBZ-lysine and the tri-peptide, @-CBZ-lysine-glycine-glycine-OH (described in Example 12 below), demonstrating the histidine-selectivity of the exemplary polymer reagent. At pH 5.0, at a reaction time of 5 hours and under the reaction conditions shown, 14% of the histidine-conjugate was formed by reaction with Reagent 2, while no detectable conjugate was formed by reaction of Reagent 2 with the single amino acid, α-CBZ-lysine, and only 1.2% conjugate was formed as a result of reaction with the tripeptide, w-CBZ-lysine-glycine-glycine-OH. At pH 5.5, at a reaction time of 5 hours and under the reaction conditions shown, 30% of the histidine-conjugate was formed by reaction with Reagent 2, while a mere 0.5% conjugate was formed by reaction of Reagent 2 with the single amino acid, α-CBZ-lysine, and only 3.1% % conjugate was formed as a result of reaction with the tripeptide, ω-CBZ-lysine-glycine-glycine-OH.

### Example 12

### CONJUGATION OF REAGENT 2 WITH TRIPEPTIDE, H₂N-LYS(α-CBZ)-GLY-GLY-OH AT PH 5.0 AND PH 5.5 AT 25ºC (1:10)—AN INVESTIGATION OF LYSINE-REACTIVITY

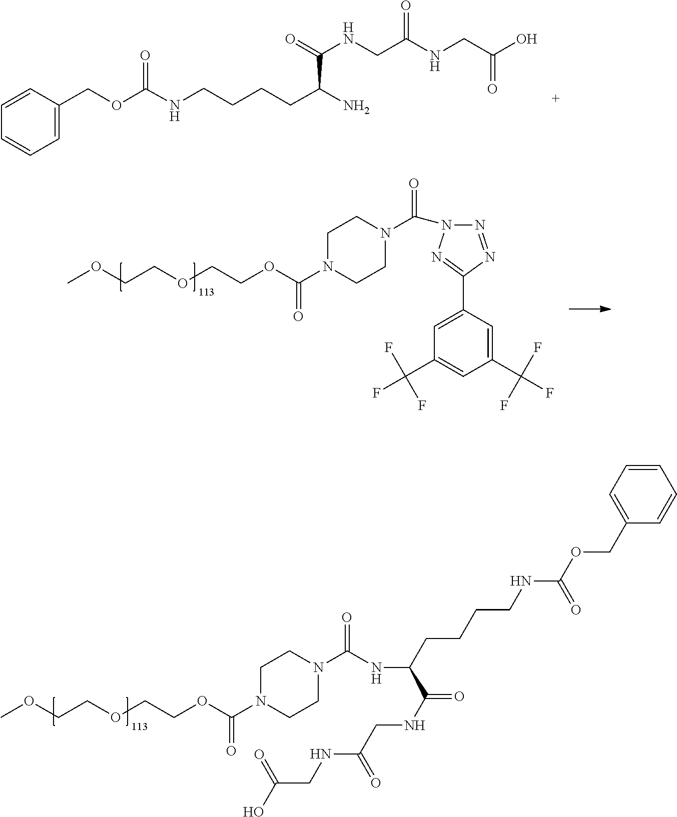

Tripeptide H₂N-Lys(a-CBZ)-Gly-Gly-OH (5.61 mg, 0.020 mmol) was dissolved in 1.0 mL of each of 100 mM phosphate buffers at pH 5.0 and 5.5, respectively. Reagent 2 (mPEG-piperazine-CO-5-(3,5-bis(trifluoromethyl)phenyl)-2H-tetrazole)) (10 mg, 0.002 mmol) was added to each solution. The resulting solutions were stirred at 25° C. and analyzed by RP-HPLC at different time intervals.

The results are summarized in Table 2 and further support the histidine-selective feature of the polymer reagents provided herein.

### Example 13

### SYNTHESIS OF ILLUSTRATIVE CONJUGATES WITH α-CBZ-HISTIDINE

13.1. Preparation of mPEG-N(CH₃)—CO-α-CBZ-Histidine, 5 kDa (Conjugate 10)

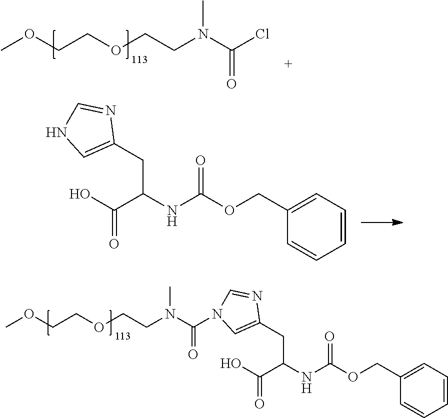

α-CBZ-Histidine (83.88 mg, 0.2900 mmol) was dissolved in 6 ml of DMF. Pyridine (0.05 mL, 0.5800 mmol) was added followed by addition of mPEG-N(CH₃)—CO—Cl (500 mg, 0.10 mmol). The reaction mixture was stirred at ambient temperature overnight.

After 20 hours reaction, the reaction product was precipitated by addition of 100 ml of MTBE. The precipitate was collected, washed with 20 ml of MTBE and dried under vacuum for 3 hours to afford 515 mg of crude product. GFC: ˜ 34% of the desired product.

The crude product was purified by ion exchange chromatography using a DEAE Sepharose FF column. Yield 115 mg; purity 98% by GFC. 1H NMR (DMSO-d₆): 8, ppm 7.97 (s,-^{ε}H Histidine, 1H), 7.53 (d, -^{δ}Histidine, 1H), 7.33 (br, Ar—H, 5H), 4.99 (s, —CH₂ benzyl, 2H), 4.27 (bs, -^{α}H Histidine, 1H), 3.51 (bs, PEG backbone), 3.24 (s, —OCH₃, 3H), 3.00 (s,—NCH₃, 3H), 2.94 (bd, -^{β}H Histidine, 1H), 2.83 (m, -^{β}Histidine, 1H).

13.2. Preparation of mPEG-Piperazine-CO— α-CBZ-Histidine, 5 kDa (Conjugate 11)

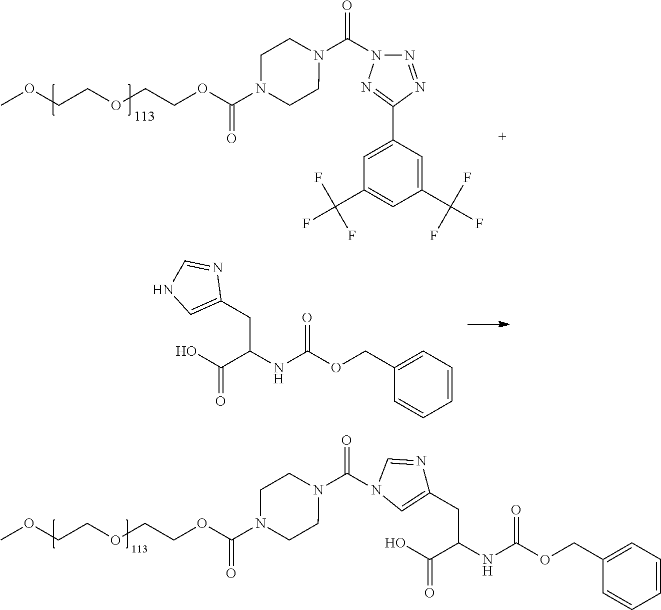

α-CBZ-Histidine (11.57 mg, 0.0400 mmol) was dissolved 2 mL of 100 mM phosphate buffer (pH 6.5), mPEG-Piperazine-CO-5-(3,5-bis(trifluoromethyl)phenyl)-2H-tetrazole (100 mg, 0.0200 mmol) was added to the α-CBZ-histidine solution. The mixture was stirred at ambient temperature overnight.

HPLC showed complete conversion of the PEG reagent to the corresponding histidine conjugate. The reaction mixture was diluted with 10% NaCl to 50 mL. The product was extracted with DCM (10 mL×3). The extract was dried with a Na₂SO4/MgSO₄ mixture, filtered, and the solvent was evaporated to dryness. The residue was precipitated with MTBE (50 ml), recovered by filtration and dried under vacuum.

The crude product (90 mg) was redissolved in 30 mL of warm IPA. After stirring for 30 minutes, the precipitate was collected and dried under vacuum to afford 52 mg of purified product.

HPLC analysis showed one peak of pure product that was not contaminated with the starting reagent, 5-(3,5-bis(trifluoromethyl)phenyl)-2H-tetrazole. The chemical structure of the product was confirmed by NMR analysis. 1H NMR (CD₂Cl₂): 8, ppm 8.51 (s, -^{ε}H Histidine, 1H), 7.34 (bs, Ar—H, 5H), 7.24 (d, -^{δ}H Histidine, 1H), 6.28 (d, —NHCO—, 1H), 5.05 (s, —CH₂ benzyl, 2H), 4.65 (t, -^{α}H Histidine, 1H), 4.24 (t, —CH₂—OCO—, 2H), 3.60 (bs, PEG backbone), 3.33 (s, —OCH₃ & -^{β}H Histidine, 4H), 3.17 (br, -^{β}H Histidine, 1H).

13.3. Preparation of mPEG-N(CH₂CF₃)—CO-α-CBZ-Histidine, 5 kDa (Conjugate 14)

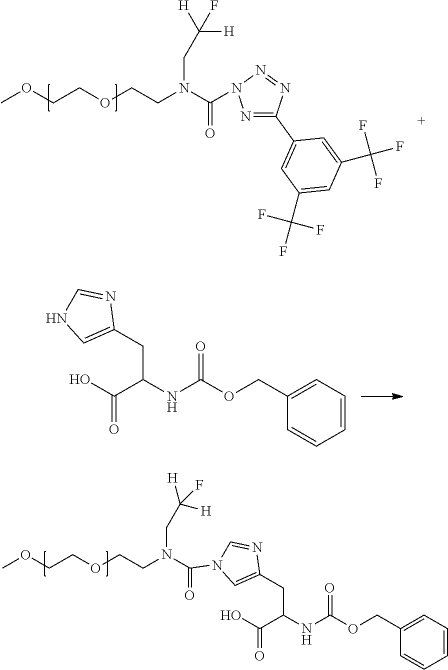

mPEG-N(CH₂CF₃)—CO-5-[3,5-bis(trifluoromethyl)phenyl-2H-tetrazole, 5K (300 mg, 0.0600 mmol) was dissolved 10 mL of 100 mM phosphate buffer (pH 5.0) containing α-CBZ-Histidine (86.79 mg, 0.3000 mmol). The reaction mixture was stirred at ambient temperature for three days.

The reaction mixture was filtered and diluted to 20 mL with D.I, water. 2 g of NaCl was added and the pH of the solution was adjusted to 3.0 by addition of 1.0 N HCl. The resulting conjugate product was extracted with DCM (10 mL×2), dried with Na₂SO4, and the extract was filtered. The solvent was evaporated to dryness and the residue was precipitated by addition of IPA (50 mL). The precipitated product was recovered by filtration, washed with 10 mL of diethyl ether and dried in vacuum for 3 hours to afford 237 mg of the crude product.

HPLC analysis showed 92% of desired product and 8% of mPEG-NH(CH₂CF₃).

13.4. Preparation of mPEG-OCO-(2-CF₃-piperazin-1-yl)-CO-α-CBZ-His 5 kDa (Conjugate 16)

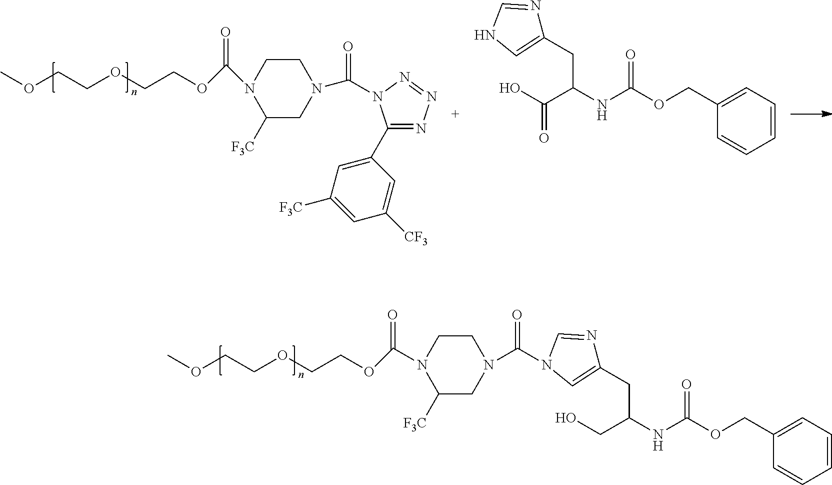

4-(CO-5-(3,5-bis(trifluoromethyl)phenyl)-2H-tetrazole)-2-trifluoromethyl-piperazin-1-yl)-mPEG-carbamate, 5 kDa (50 mg, 0.0100 mmol) was dissolved 5 mL of 100 mM phosphate buffer (pH 5.5) containing α-CBZ-Histidine (28.93 mg, 0.1000 mmol). The reaction mixture was stirred at ambient temperature over the weekend.

HPLC showed 91% of the desired conjugate, 9% of mPEG-CF₃-piperidine, 5 kDa, and no starting PEG reagent. The reaction mixture was filtered, and the filtrate was concentrated using Vivaspin centrifugal concentrators (MWCO 3000). Following concentration, the solution was lyophilized providing 35 mg of the solid product having 88% purity by HPLC.

13.5. Preparation of mPEG-OCO—NH-(piperidin-4-yl)-CO-α-CBZ-His 5 kDa (Conjugate 17)

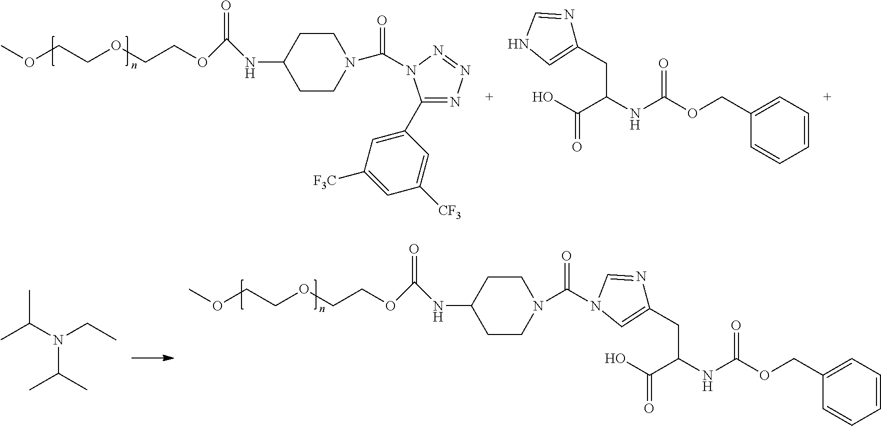

α-CBZ-Histidine (11.57 mg, 0.0400 mmol) was dissolved in 5 mL of DMF. (1-(CO-5-(3,5-bis(trifluoromethyl)phenyl)-2H-tetrazole)-piperidin-4-yl)-mPEG-carbamate 5 kDa (100 mg, 0.0200 mmol) and N,N-diisopropylethylamine (0.02 mL, 0.1200 mmol) were added. The reaction mixture was stirred at ambient temperature overnight.

A sample, checked by HPLC indicated the presence of ˜ 3% unreacted starting material. N,N-diisopropylethylamine (0.02 mL, 0.1200 mmol) was added and reaction mixture was stirred at ambient temperature overnight. DCM was evaporated and the residue was precipitated with MTBE (70 mL, cold). The precipitate was collected and dried under vacuum overnight to afford 92 mg of the desired conjugate product having a purity of 98.3% by HPLC.

13.6. Preparation of mPEG-OCO—NH—CH₂-(piperidin-4-yl)-α-CBZ-His 5 kDa (Conjugate 18)

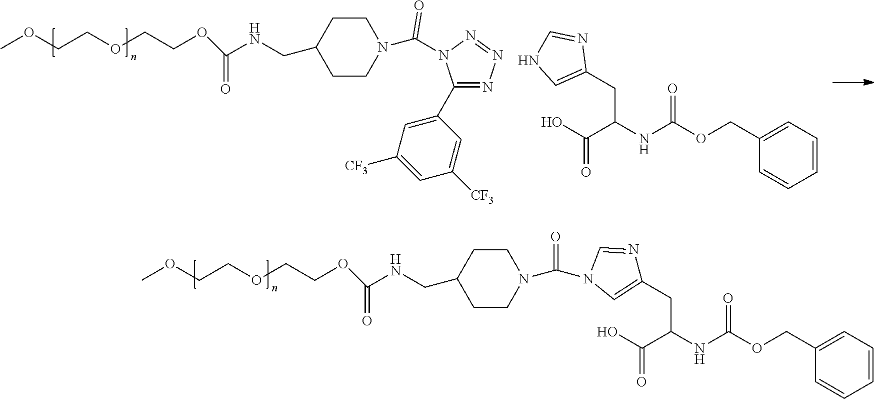

(1-(CO-5-(3,5-bis(trifluoromethyl)phenyl)-2H-tetrazole)-piperidin-4-yl)-methyl-mPEG-carbamate 5 k (10 mg, 0.002 mmol) was dissolved 1 mL of 100 mM phosphate buffer (pH 6.5) containing α-CBZ-Histidine (5.79 mg; 0.0200 mmol). The solution was filtered and stirred at room temperature. The reaction progress was monitored by RP-HPLC at different time intervals. After 5 hours, ˜ 36% of conjugate had formed. After 10 hours, ˜68% of conjugate had formed. After 15 hours, 87% of the conjugate had formed.

The above reaction was repeated three times more at different pHs (5.0, 5.5 and 6.0) and the reaction mixtures were combined. HPLC analysis showed 88% of the desired product and 12% of mPEG mPEG-OCO—NH—CH₂-(piperidin-4-yl).

The combined solution was dialyzed against water (MECO 3K) (3L×3). Following dialysis, the solution was passed through a POROS 50HS column (5 mL) to remove of the mPEG-OCO—NH—CH₂-(piperidin-4-yl). The eluent was concentrated via centrifugation filtration. The pH of concentrated solution was adjusted to 7.0 by addition of 0.1N NaOH and the solution was lyophilized overnight to afford 35 mg of the desired product having 97% purity by HPLC.

Additional conjugates were similarly prepared.

### Example 14

### HYDROLYTIC STABILITY OF CONJUGATES 10-18 IN PBS BUFFER AT PH 7.4, 37° C.

14.1. Determination of hydrolytic stability of Conjugate 10 (mPEG-N(CH₃)—CO-Histidine (α-CBZ)-5 kDa in PBS buffer at pH 7.4, 37° C.

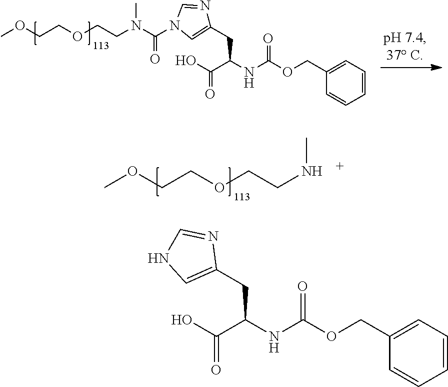

mPEG-N(CH₃)—CO-Histidine (α-CBZ)-5 kDa (10.0 mg) was dissolved in 1.0 mL of PBS buffer pH 7.4. The reaction mixture was incubated at 37° C. and periodically analyzed by HPLC. After 10 days, only 1.8% of the conjugate had undergone hydrolysis to produce mPEG-NH(CH₃).

14.2. Determination of hydrolytic stability of Conjugate 11, 14, 16, 17, and 18 in PBS buffer at pH 7.4, 37° C.

The hydrolytic stability of Conjugates 11, 14, 16, 17, and 18 was determined following the method described above for Conjugate 10. The stability results are summarized in Table 3 and shown in FIG. 4.

TABLE 3
Stability of PEG conjugates with α-CBZ-Histidine in PBS buffer, pH 7.4,
37° C. (Percent Hydrolysis at Day 10)

| | | | % |
|---|---|---|---|
| Conjugate | | | Hydrolysis |
| Identifier | Conjugate Name | Structure | at day 10 |
| Conjugate 10 | mPEG-N(CH₃)-C-α-CBZ- His | 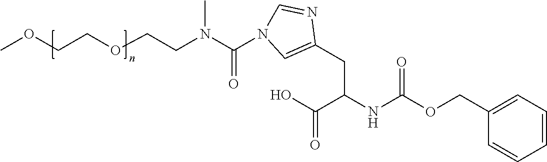 | 1.9 |
| Conjugate 11 | mPEG-Piperazine-CO-α- CBZ-His | 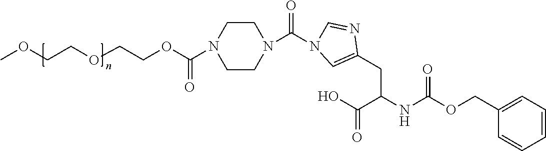 | 15.6 |
| Conjugate 12 | mPEG-N(CH₂CH₂F)-CO- α-CBZ-His | 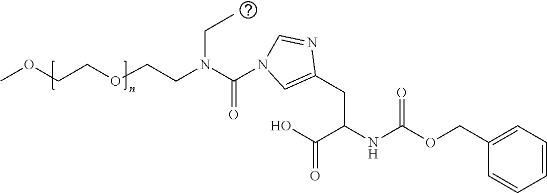 | |
| Conjugate 13 | mPEG-N(CH₂CHF₂)-CO- α-CBZ-His | 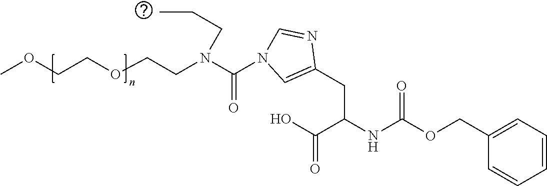 | |
| Conjugate 14 | mPEG-N(CH₂CF₃)-CO- α-CBZ-His | 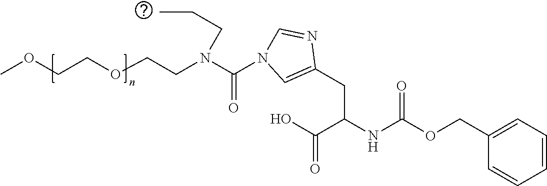 | 26.5 |
| Conjugate 15 | mPEG-N(C₆H₅)-CO-α- CBZ-His | 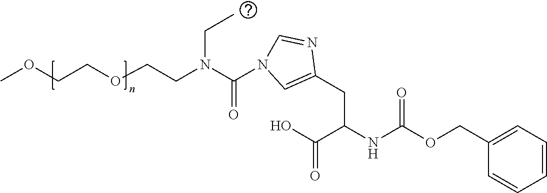 | |
| Conjugate 16 | mPEG-OCO-(2-CF₃-piperazin- 1-yl)-CO-α-CBZ-His | 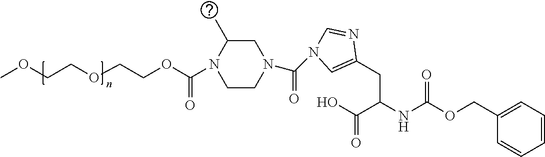 | 46.0 |
| Conjugate 17 | mPEG-OCO-NH-(piperidin-4- yl)-CO-α-CBZ-His | 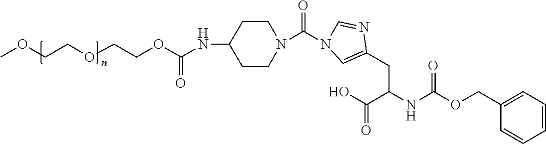 | 1.8 |
| Conjugate 18 | mPEG-OCO-NH-CH2- (piperidin-4-yl)-α-CBZ-His | 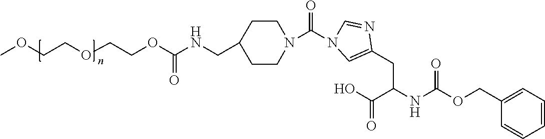 | 2.7 |

Conjugates formed by reaction of a model histidine-comprising compound, α-CBZ-histidine, with exemplary histidine-selective polymer reagents as provided herein, are relatively stable in aqueous buffer at pHs up to about 8.0. The urea-linked conjugates that were evaluated and that demonstrated the highest degree of hydrolytic stability relative to the other conjugates, Conjugates 10, 17 and 18, are those wherein R₁ optionally in combination with R₂, when present, in reference to the general formula,

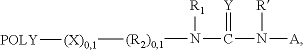

are methyl and piperidine. Conjugate 11, having a moderate degree of hydrolytic stability relative to the other conjugates tested, comprises a piperazine moiety (formed by R₁ and R₂, taken together in combination with N), in addition to linker X, ˜OC(O)˜. Conjugate 14, similarly having a moderate degree of hydrolytic stability relative to the other conjugates tested, possesses as R₁, ˜CH₂CH₂CF₃. Finally, the conjugate showing the highest degree of hydrolysis under the test conditions, Conjugate 16, comprises a trifluoromethyl-substituted piperazine (formed by R₁ and R₂, taken together in combination with N) in addition to linker X, ˜OC(O)˜. The hydrolytic stability of the resulting conjugates (at a given pH and temperature, such as, for example, under physiological conditions or conditions mimicking physiological conditions), is influenced by the selection of R₁, R₂ and X, where the presence of electron withdrawing groups appears to contribute to a greater degree of hydrolysis. Thus, conjugates as generally described herein, and comprising one or more electron-withdrawing groups or atoms within the overall spacer, ˜(X)_{0.1}—(R₂)_{0,1}-NR₁˜ are expected to exhibit a diminished hydrolytic stability when compared to conjugates absent such electron withdrawing moieties. Thus, as described previously, polymer reagents (and thus the conjugates formed by reaction therewith) can be designed to possess optimal reactivity, selectivity and conjugate stability for reaction with a desired target molecule or surface by appropriate selection of polymer reagent components such as X, R₂ and R_{1.}

### Example 15

### HISTIDINE SELECTIVITY OF EXEMPLARY CONJUGATES—REACTION WITH HYDROXYLAMINE AT PH 7.4, 25° C.

FIGS. 14A and 14B provide further evidence of the histidine-selectivity of exemplary polymer reagents provided herein, as illustrated by reaction of their corresponding histidine-linked conjugates with hydroxylamine. The data described below is representative of an indirect method for evaluating covalent attachment of a polymer reagent as provided herein with a histidine residue of an amino acid, peptide, polypeptide or other active histidine containing molecule, by displacement of the histidine-attached molecule resulting from reaction of the histidine-linked polymer conjugate with hydroxylamine.

More particularly, FIG. 14A is a plot illustrating reaction of a histidine conjugate prepared by reaction of Reagent 1 with model compound, α-CBZ-His, with hydroxylamine at pH 7.4, 25° C. The plot shows percent of mPEG-N(CH₃)—CO-His(α-CBZ) conjugate remaining over time. Histidine conjugates such as the illustrative conjugates tested, when treated with hydroxylamine, undergo a reverse reaction such that the unconjugated histidine compound is released. In contrast, hydroxylamine does not typically react with a lysine-linked conjugate to cleave the lysine-polymer linkage to thereby release the parent lysine compound. By approximately 22 hours, 100% of the conjugate has disappeared with release of α-CBZ-His.

Similarly, FIG. 14B is a plot illustrating reaction of a histidine conjugate prepared by reaction of Reagent 2 with model compound, α-CBZ-His, with hydroxylamine at pH 7.3, 25° C. The plot shows percent of mPEG-piperazine —CO-His(α-CBZ) conjugate remaining over time, wherein at approximately 42 hours, only 4% of the intact conjugate remained.

It is claimed:

1. A polymer reagent of Formula I: 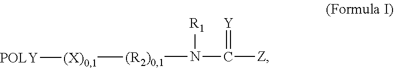
wherein:
POLY is a water-soluble polymer;
X is a linker moiety;
R₁ is an organic radical and may form a nitrogen-containing heterocycle when taken together with R₂;
R₂, when present, taken together with R₁ forms a nitrogen-containing heterocycle;
Y is selected from O and S;
Z is a leaving group.

2. A polymer reagent of claim 1, wherein R₁ is an organic radical selected from substituted and unsubstituted alkyl, substituted and unsubstituted cycloalkyl, substituted and unsubstituted alkenyl, substituted and unsubstituted cycloalkenyl, substituted and unsubstituted alkynyl, substituted and unsubstituted heteroalkyl, substituted and unsubstituted cycloheteroalkyl, substituted and unsubstituted aryl, substituted and unsubstituted aralkyl, substituted and unsubstituted heteroaryl, and substituted and unsubstituted heteroaralkyl.

3. The polymer reagent of claim 1, wherein R₁ is an organic radical selected from alkyl, cycloalkyl, alkenyl, cycloalkenyl, alkynyl, heteroalkyl, cycloheteroalkyl, aryl, aralkyl, heteroaryl, and heteroaralkyl, each optionally substituted with one or more substituents independently selected from the group consisting of halo, alkylhalo, hydroxy, alkylhydroxy, alkoxy, amino, alkylamino, sulfhydryl, alkylsulfhydryl, nitro, alkylnitro, cyano, alkylcyano, thiocyano, alkylthiocyano, imino, alkylimino, carbamate, alkylcarbamate, phosphate, alkylphosphate, alkylcarbonyl, carboxamide, alkylcarboxamide, alkoxycarbonyl, thioalkyl, thioester, and alkylthioester.

4. The polymer reagent of claim 3, wherein R₁ is selected from the group consisting of lower alkyl, halo-substituted lower alkyl, benzyl, halo-substituted benzyl and nitro-substituted benzyl, wherein the benzyl ring has from one to five halo-substituents.

5. The polymer reagent of claim 3 or 4, wherein the halo substituent is fluoro.

6. The polymer reagent of claim 1, wherein R₂ is absent.

7. The polymer reagent of claim 1, wherein R₂ is present.

8. The polymer reagent of claim 1 or claim 7, wherein R₂, taken together with R₁ forms a nitrogen-containing heterocycle containing 4, 5, 6, or 7 heterocycle ring atoms.

9. The polymer reagent of claim 8, wherein the nitrogen-containing heterocycle contains from one to three nitrogen atoms.

10. The polymer reagent of claim 1 or claim 7, wherein R₂ together with R₁ forms a nitrogen-containing heterocycle selected from group consisting of azetidine, substituted azetidine, diazetidine, substituted diazetidine, pyrrolidine, substituted pyrrolidine, imidazolidine, substituted imidazolidine, piperidine, substituted piperidine, morpholine, substituted morpholine, diazinanes, substituted diazinanes, triazinanes, substituted triazinanes, azepanes, substituted azepanes, diazepanes and substituted diazepanes.

11. The polymer reagent of any one of claims 7, 8, 9, and 10, wherein R₂ together with R₁ forms a piperidine or a substituted piperidine.

12. The polymer reagent of any one of claims 7, 8, 9, and 10, wherein R₂ taken together with R₁ forms a diazinane or a substituted diazinane.

13. The polymer reagent of claim 12, wherein the diazinane or substituted diazinane is piperazine or substituted piperazine, respectively.

14. The polymer reagent of any one of claims 7-13, wherein the nitrogen-containing heterocycle is optionally substituted with one or more substituents selected from the group consisting of alkyl, substituted alkyl, alkenyl, substituted alkenyl, alkynyl, substituted alkynyl, heteroalkyl, substituted heteroalkyl, aralkyl, substituted aralkyl, halo, alkylhalo, hydroxy, alkylhydroxy, alkoxy, amino, alkylamino, sulfhydryl, alkylsulfhydryl, nitro, alkylnitro, cyano, alkylcyano, thiocyano, alkylthiocyano, imino, alkylimino, carbamate, alkylcarbamate, phosphate, alkylphosphate, alkylcarbonyl, carboxamide, alkylcarboxamide, alkoxycarbonyl, thioalkyl, thioester, and alkylthioester.

15. The polymer reagent of any one of claims 7-14, wherein R₂ taken together with R₁ forms a nitrogen-containing heterocycle that is (i) unsubstituted or (ii) is substituted at one or more ring positions with lower alkyl, substituted lower alkyl, aralkyl, or substituted aralkyl.

16. The polymer reagent of claim 15, wherein R₂ taken together with R₁ forms a nitrogen-containing heterocycle that is substituted at one or more ring positions with lower alkyl, halo-substituted lower alkyl, aralkyl, or halo-substituted aralkyl.

17. The polymer reagent of any one of claims 7-16, wherein the nitrogen-containing heterocycle is mono- or di-substituted.

18. The polymer reagent of any one of claims 1-16, wherein X is absent.

19. The polymer reagent of any one of claims 1-14, wherein X is present.

20. The polymer reagent of claim 19, wherein X is selected from —O—, —S—, —NH—, —C(O)—, —O—C(O)—, —C(O)—O—, —C(O)—NH—, —NH—C(O)—NH—, —O—C(O)—NH—, —C(S)—, —CH₂—, —CH₂—CH₂—, —CH₂—CH₂—CH₂—, —CH₂—CH₂—CH₂—CH₂, —O—CH₂—, —CH₂—O—, —O—CH₂—CH₂—, —CH₂—O—CH₂—, —CH₂—CH₂—O—, —O—CH₂—CH₂—CH₂—, —CH₂—O—CH₂—CH₂—, —CH₂—CH₂—O—CH₂—, —CH₂—CH₂—CH₂—O—, —O—CH₂—CH₂—CH₂—CH₂—, —CH₂—O—CH₂—CH₂—CH₂—, —CH₂—CH₂—O—CH₂—CH₂—, —CH₂—CH₂—CH₂—O—CH₂—, —CH₂—CH₂—CH₂—CH₂—O—, —C(O)—NH—CH₂—, —C(O)—NH—CH₂—CH₂—, —CH₂—C(O)—NH—CH₂—, —CH₂—CH₂—C(O)—NH—, —C(O)—NH—CH₂—CH₂—CH₂—, —CH₂—C(O)—NH—CH₂—CH₂—, —CH₂—CH₂—C(O)—NH—CH₂ —, —CH₂—CH₂—CH₂—C(O)—NH—, —C(O)—NH—CH₂—CH₂—CH₂—CH₂—, —CH₂—C(O)—NH—CH₂—CH₂—CH₂—, —CH₂—CH₂—C(O)—NH—CH₂—CH₂—, —CH₂—CH₂—CH₂—C(O)—NH—CH₂—, —CH₂—CH₂—CH₂—C(O)—NH—CH₂—CH₂—, —CH₂—CH₂—CH₂—CH₂—C(O)—NH—, —C(O)—O—CH₂—, —CH₂—C(O)—O—CH₂—, —CH₂—CH₂C(O)—O—CH₂—, —C(O)—O—CH₂—CH₂—, —NH—C(O)—CH₂—, —CH₂—NH—C(O)—CH₂—, —CH₂—CH₂—NH—C(O)—CH₂—, —NH—C(O)—CH₂—CH₂—, —CH₂—NH—C(O)—CH₂—CH₂—, —CH₂—CH₂—NH—C(O)—CH₂—CH₂—, —C(O)—NH—CH₂—, —C(O)—NH—CH₂—CH₂—, —O—C(O)—NH—CH₂—, —O—C(O)—NH—CH₂CH₂, —O—C(O)—NH—CH₂—CH₂—CH₂—, —NH—CH₂—, —NH—CH₂—CH₂—, —CH₂—NH—CH₂—, —CH₂—CH₂—NH—CH₂—, C(O)CH₂—, —C(O)—CH₂—CH₂—, —CH₂—C(O)—CH₂—, —CH₂—CH₂C(O)—CH₂—, —CH₂—CH₂—C(O)—CH₂—CH₂—, —CH₂—CH₂—C(O)—, —CH₂—CH₂—CH₂—C(O)—NH—CH₂—CH₂—NH—, —CH₂—CH₂—CH₂—C(O)—NH—CH₂—CH₂—NH—C(O)—, —CH₂—CH₂—CH₂—C(O)—NH—CH₂—CH₂—NH—C(O)—CH₂—, —CH₂—CH₂—CH₂—C(O)—NH—CH₂—CH₂—NH—C(O)—CH₂—CH₂—, —O—C(O)—NH—(CH₂)₀₋₆—(OCH₂CH₂)₀₋₂—, C(O)—NH—(CH₂)₁₋₆—NH—C(O)—, —NH—C(O)—NH—(CH₂)₁₋₆—NH—C(O)—, —O—C(O)—CH₂—, —O—C(O)—CH₂—CH₂—, —O—C(O)—CH₂—CH₂—CH₂ —. , and combinations of any one or more of the foregoing.

21. The polymer reagent of claim 19, wherein X is ˜(CH₂)ₐ(O)_{b}[C(O)](NH)_{d}(CH₂)˜, wherein: a is 0-6; b is 0,1; c is 0,1; d is 0,1; and e is 0-6, wherein at least one of a, b, c, d, and e is a positive integer.

22. The polymer reagent of claim 20, wherein X is selected from —O—C(O)—, —O—C(O)—NH—, and —O—C(O)—NH—CH₂ —.

23. The polymer reagent of any one of claims 1-22, wherein Y is O.

24. The polymer reagent of any one of claims 1-22, wherein Y is S.

25. The polymer reagent of any one of claims 1-24, wherein POLY is a water-soluble polymer selected from the group consisting of poly(alkylene oxide), poly(vinyl pyrrolidone), poly(vinyl alcohol), polyoxazoline, poly(acryloylmorpholine), polyacrylic acid, polyacrylamides, N-(2-hydroxypropyl) methylacrylamide, divyinyl ether-maleic anhydride, polyphosphates, polyphosphazenes, and co-polymers and ter-polymers thereof.

26. The polymer reagent of claim 25, wherein POLY is a poly(alkylene oxide).

27. The polymer reagent of claim 26, wherein POLY is a poly(ethylene glycol).

28. The polymer reagent of claim 27, wherein the poly(ethylene glycol) is terminally capped with an end-capping moiety selected from the group consisting of hydroxy, alkoxy, substituted alkoxy, alkenoxy, substituted alkenoxy, alkynoxy, substituted alkynoxy, aryloxy and substituted aryloxy.

29. The polymer reagent of claim 28, wherein the poly(ethylene glycol) is end-capped with methoxy.

30. The polymer reagent of any one of claims 1-29, wherein POLY is a water-soluble polymer that is linear, branched, or multi-armed.

31. The polymer reagent of any one of the foregoing claims, wherein POLY has a weight average molecular weight from about 100 daltons to about 100,000 daltons.

32. The polymer reagent of claim 31, wherein POLY has a weight average molecular weight in a range of from about 200 daltons to about 80,000 daltons, or from about 500 daltons to about 70,000 daltons, or from about 1,000 daltons to about 60,000 daltons, or from about 5,000 daltons to about 25,000 daltons, or from about 5,000 daltons to about 30,000 daltons, or from about 5,000 daltons to about 50,000 daltons, or from about 10,000 daltons to about 60,000 daltons, or from about 10,000 daltons to about 50,000 daltons, or from about 20,000 daltons to about 50,000 daltons, or from about 20,000 daltons to about 40,000 daltons, or from about 20,000 daltons to about 80,000 daltons.

33. The polymer reagent of claim 31, wherein POLY has a weight average molecular weight selected from the group consisting of 200 daltons, 300 daltons, 400 daltons, 500 daltons, 750 daltons, 1,000 daltons, 2,500 daltons, 3,000 daltons, 5,000 daltons, 7500 daltons, 10,000 daltons, 15,000 daltons, 20,000 daltons, 25,000 daltons, 30,000 daltons, 40,000 daltons, 50,000 daltons, 55,000 daltons, and 60,000 daltons.

34. The polymer reagent of any one of claims 1-33, wherein Z is selected from the group consisting of tetrazoles, isocyanates, isothiocyanates, N-hydroxysuccinimide, acyl azide, fluorophenol, benzotriazoles, nitrophenols, and triazoles.

35. The polymer reagent of claim 34, wherein Z is a tetrazole leaving group.

36. The polymer reagent of claim 35, wherein Z is a phenyl tetrazole.

37. The polymer reagent of claim 36, wherein Z is a phenyl tetrazole having a structure: 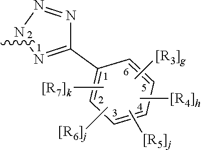
wherein g, h, i, j, and k is each independently 0 or 1 (wherein 0 indicates absence and 1 indicates presence), and R₃, R₄, R₅, R₆ and R₇ is each independently selected from the group consisting of alkyl, substituted alkyl, alkenyl, substituted alkenyl, alkynyl, substituted alkynyl, heteroalkyl, substituted heteroalkyl, aralkyl, substituted aralkyl, halo, alkylhalo, hydroxy, alkylhydroxy, alkoxy, amino, alkylamino, sulfhydryl, alkylsulfhydryl, nitro, alkylnitro, cyano, alkylcyano, thiocyano, alkylthiocyano, imino, alkylimino, carbamate, alkylcarbamate, phosphate, alkylphosphate, alkylcarbonyl, carboxamide, alkylcarboxamide, alkoxycarbonyl, thioalkyl, thioester, and alkylthioester.

38. The polymer reagent of claim 37, therein the phenyl tetrazole has a single substituent on the phenyl ring.

39. The polymer reagent of claim 38, wherein the single substituent is at carbon 2 of the phenyl ring.

40. The polymer reagent of claim 38, wherein the single substituent is at carbon 3 of the phenyl ring.

41. The polymer reagent of claim 38, wherein the single substituent is at carbon 4 of the phenyl ring.

42. The polymer reagent of claim 37, therein the phenyl tetrazole has two substituents on the phenyl ring.

43. The polymer reagent of claim 42, wherein the two phenyl substituents are at C2 and C3.

44. The polymer reagent of claim 42, wherein the two phenyl substituents are at C2 and C4.

45. The polymer reagent of claim 42, wherein the two phenyl substituents are at C3 and C5.

46. The polymer reagent of claim 42, wherein the two phenyl substituents are at C3 and C4.

47. The polymer reagent of claim 42, wherein the two phenyl substituents are at C2 and C6.

48. The polymer reagent of claim 37, therein the phenyl tetrazole has three substituents on the phenyl ring.

49. The polymer reagent of claim 48, wherein the three phenyl substituents are at C2, C3 and C4.

50. The polymer reagent of claim 48, wherein the three phenyl substituents are at C2, C3, and C5.

51. The polymer reagent of claim 48, wherein the three phenyl substituents are at C2, C3 and C6.

52. The polymer reagent of claim 48, wherein the three phenyl substituents are at C2, C4, and C6.

53. The polymer reagent of claim 48, wherein the three phenyl substituents are at C3, C4, and C5.

54. The polymer reagent of claim 48, wherein the three phenyl substituents are at C3, C4, and C6.

55. The polymer reagent of claim 37, wherein the phenyl tetrazole has four substituents on the phenyl ring.

56. The polymer reagent of claim 55, wherein the four phenyl substituents are at C2, C3, C4, and C5.

57. The polymer reagent of claim 55, wherein the four phenyl substituents are at C2, C3, C5, and C6.

58. The polymer reagent of claim 55, wherein the four phenyl substituents are at C2, C3, C4, and C6.

59. The polymer reagent of claim 37, wherein the phenyl tetrazole has five substituents on the phenyl ring.

60. The polymer reagent of any one of claims 42 to 59, wherein the substituents on the phenyl ring are the same.

61. The polymer reagent of any one of claims 43 to 60, wherein one or more of the substituents on the phenyl ring are different.

62. The polymer reagent of any one of claims 36-61, wherein the one or more substituents are trifluoromethyl.

63. The polymer reagent of any one of claims 1, 32 and 33 selected from the group consisting of: 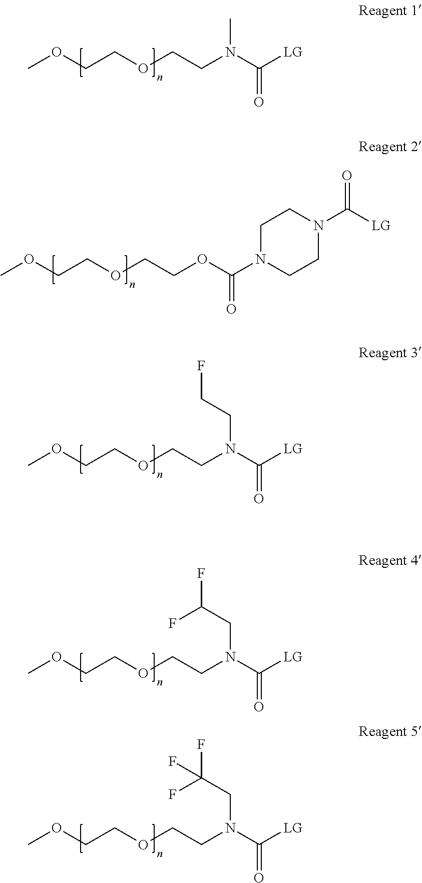 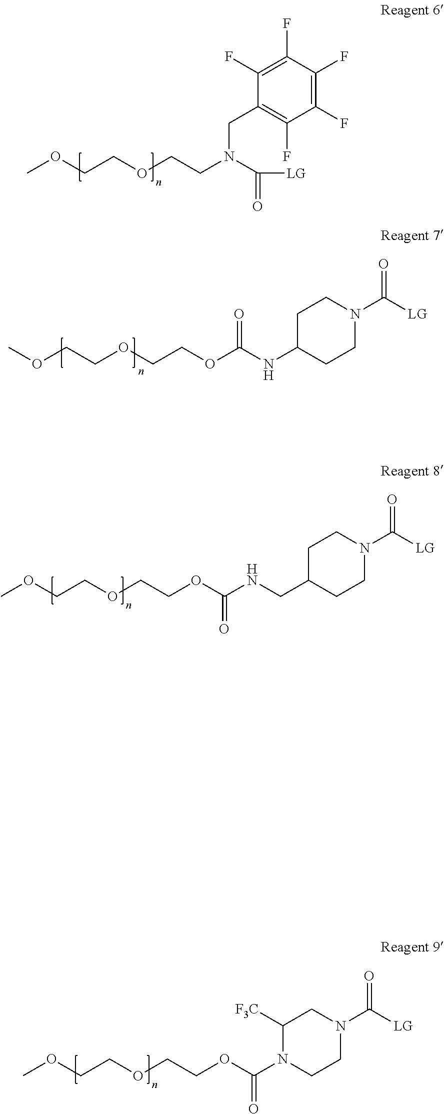
wherein each (n) is independently in a range selected from the group consisting of: from about 2 to about 2,273; from about 4 to about 1800; from about 11-1590; from about 23 to about 1363; from about 113 to about 568; from about 113 to about 682; from about 113 to about 1136; from about 227 to about 1363; from about 227 to about 1136; from about 454 to about 1136; from about 454 to about 909; and from about 454 to about 1818; and LG is a leaving group.

64. The polymer reagent of any one of claims 1, 32 and 33 selected from the group consisting of: 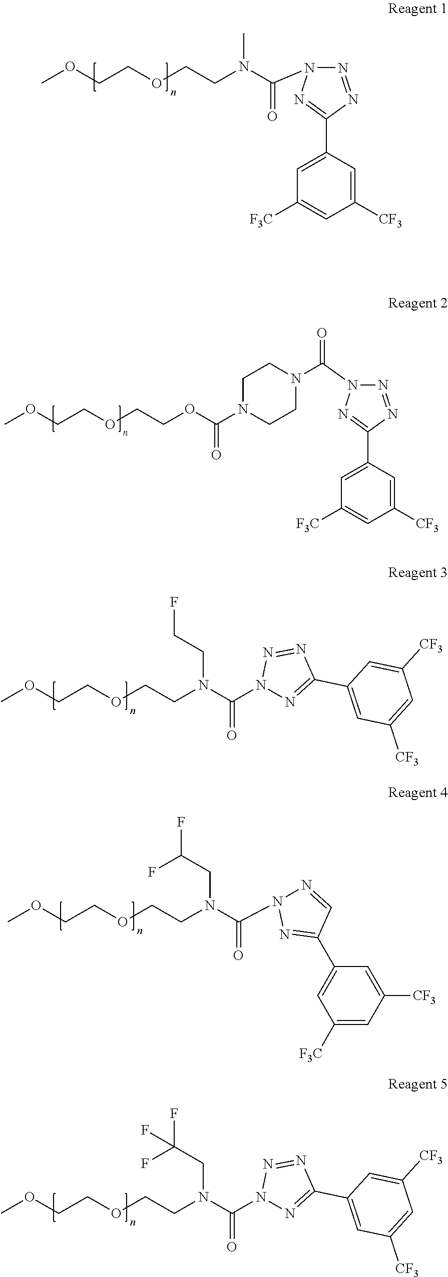 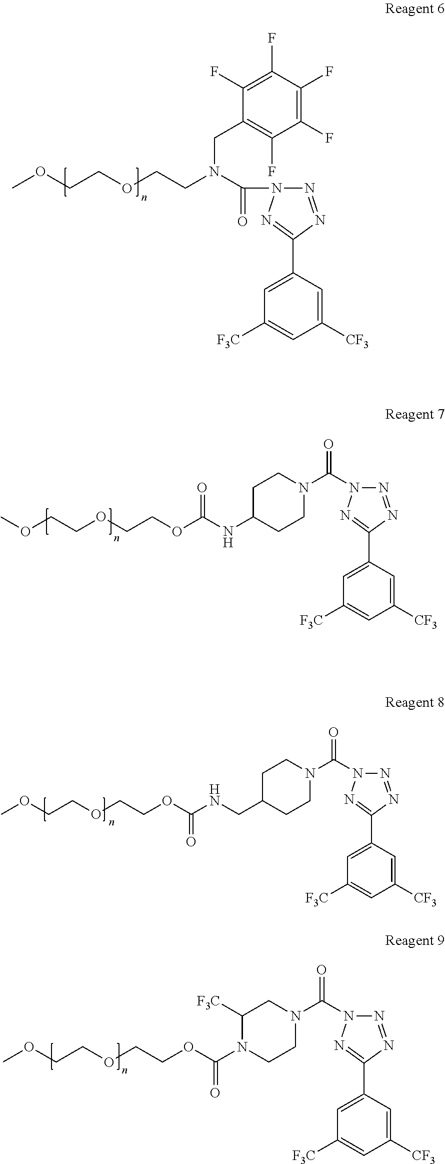
wherein each (n) is independently in a range selected from the group consisting of: from about 2 to about 2,273; from about 4 to about 1800; from about 11-1590; from about 23 to about 1363; from about 113 to about 568; from about 113 to about 682; from about 113 to about 1136; from about 227 to about 1363; from about 227 to about 1136; from about 454 to about 1136; from about 454 to about 909; and from about 454 to about 1818.

65. A conjugate prepared by reacting a polymer reagent of any one of claims 1-64 with an active agent comprising one or more amino groups under conditions effective to promote conjugation between the one or more amino groups of the active agent and the polymer reagent.

66. The conjugate of claim 65, wherein the active agent is selected from a protein, a peptide, and a small molecule.

67. The conjugate of either claim 65 or claim 66, wherein the active agent comprises one or more histidine residues comprising an amino group (“histidine amino group”), and the one or more histidine amino groups are covalently attached to the ˜C(Y)˜ carbon of the polymer reagent.

68. A conjugate having a formula: 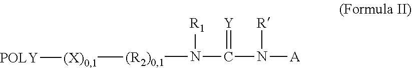
wherein:
POLY is a water-soluble polymer;
X is a linker moiety;
R₁ is an organic radical and may form a nitrogen-containing heterocycle when taken together with R₂;
R₂, when present, taken together with R₁ forms a nitrogen-containing heterocycle;
Y is selected from O and S;
R′ is H or an organic radical; and
A-N—R′ is an active agent comprising an amino group.

69. The conjugate of claim 68 having a formula: 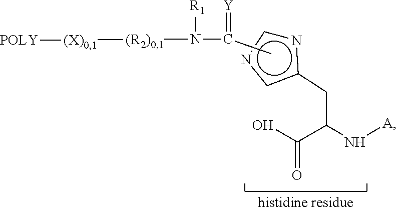
wherein 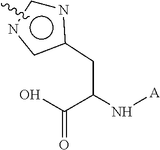
is an active agent comprising a histidine residue.

70. The conjugate of claim 69, wherein the active agent is a peptide or a protein comprising a histidine residue.

71. The conjugate of any one of claims 68 to 70, wherein R₁ is an organic radical selected from substituted and unsubstituted alkyl, substituted and unsubstituted cycloalkyl, substituted and unsubstituted alkenyl, substituted and unsubstituted cycloalkenyl, substituted and unsubstituted alkynyl, substituted and unsubstituted heteroalkyl, substituted and unsubstituted cycloheteroalkyl, substituted and unsubstituted aryl, substituted and unsubstituted aralkyl, substituted and unsubstituted heteroaryl, and substituted and unsubstituted heteroaralkyl.

72. The conjugate of any one of any one of claims 68 to 70, wherein R₁ is an organic radical selected from alkyl, cycloalkyl, alkenyl, cycloalkenyl, alkynyl, heteroalkyl, cycloheteroalkyl, aryl, aralkyl, heteroaryl, and heteroaralkyl, each optionally substituted with one or more substituents independently selected from the group consisting of halo, alkylhalo, hydroxy, alkylhydroxy, alkoxy, amino, alkylamino, sulfhydryl, alkylsulfhydryl, nitro, alkylnitro, cyano, alkylcyano, thiocyano, alkylthiocyano, imino, alkylimino, carbamate, alkylcarbamate, phosphate, alkylphosphate, alkylcarbonyl, carboxamide, alkylcarboxamide, alkoxycarbonyl, thioalkyl, thioester, and alkylthioester.

73. The conjugate of claim 72, wherein Riis selected from the group consisting of lower alkyl, halo-substituted lower alkyl, benzyl, halo-substituted benzyl and nitro-substituted benzyl, wherein the benzyl ring has from one to five halo-substituents.

74. The conjugate of claim 72 or 73, wherein the halo substituent is fluoro.

75. The conjugate of claim 68, wherein R₂ is absent.

76. The conjugate of claim 68, wherein R₂ is present.

77. The conjugate of claim 68 or 76, wherein R₂, taken together with R₁ forms a nitrogen-containing heterocycle containing 4, 5, 6, or 7 heterocycle ring atoms.

78. The conjugate of claim 77, wherein the nitrogen-containing heterocycle contains from one to three nitrogen atoms.

79. The conjugate of claim 68 or 76, wherein R₂ together with R₁ forms a nitrogen-containing heterocycle selected from group consisting of azetidine, substituted azetidine, diazetidine, substituted diazetidine, pyrrolidine, substituted pyrrolidine, imidazolidine, substituted imidazolidine, piperidine, substituted piperidine, diazinanes, substituted diazinanes, triazinanes, substituted triazinanes, azepanes, substituted azepanes, diazepanes and substituted diazepanes.

80. The conjugate of any one of claims 76 to 79, wherein R₂ together with R₁ forms a piperidine or a substituted piperidine.

81. The conjugate of any one of claims 76 to 79, wherein R₂ taken together with R₁ forms a diazinane or a substituted diazinane.

82. The conjugate of claim 81, wherein the diazinane or substituted diazinane is piperazine or substituted piperazine, respectively.

83. The conjugate of any one of claims 76 to 82, wherein the nitrogen-containing heterocycle is optionally substituted with one or more substituents selected from the group consisting of alkyl, substituted alkyl, alkenyl, substituted alkenyl, alkynyl, substituted alkynyl, heteroalkyl, substituted heteroalkyl, aralkyl, substituted aralkyl, halo, alkylhalo, hydroxy, alkylhydroxy, alkoxy, amino, alkylamino, sulfhydryl, alkylsulfhydryl, nitro, alkylnitro, cyano, alkylcyano, thiocyano, alkylthiocyano, imino, alkylimino, carbamate, alkylcarbamate, phosphate, alkylphosphate, alkylcarbonyl, carboxamide, alkylcarboxamide, alkoxycarbonyl, thioalkyl, thioester, and alkylthioester.

84. The conjugate of any one of claims 76 to 83, wherein R₂ taken together with R₁ forms a nitrogen-containing heterocycle that is (i) unsubstituted or (ii) is substituted at one or more ring positions with lower alkyl, substituted lower alkyl, aralkyl, or substituted aralkyl.

85. The conjugate of claim 84, wherein R₂ taken together with R₁ forms a nitrogen-containing heterocycle that is substituted at one or more ring positions with lower alkyl, halo-substituted lower alkyl, aralkyl, or halo-substituted aralkyl.

86. The conjugate of any one of claims 76 to 85, wherein the nitrogen-containing heterocycle is mono- or di-substituted.

87. The conjugate of any one of claims 76 to 85, wherein X is absent.

88. The conjugate of any one of claims 76 to 83, wherein X is present.

89. The conjugate of claim 87, wherein X is selected from —O—, —S—, —NH—, —C(O)—, —O—C(O)—, —C(O)—O—, —C(O)—NH—, —NH—C(O)—NH—, —O—C(O)—NH—, —C(S)—, —CH₂—, —CH₂—CH₂—, —CH₂—CH₂—CH₂—, —CH₂—CH₂—CH₂—CH₂, —O—CH₂—, —CH₂—O—, —O—CH₂—CH₂—, —CH₂—O—CH₂—, —CH₂—CH₂—O—, —O—CH₂—CH₂—CH₂—, —CH₂—O—CH₂—CH₂—, —CH₂—CH₂—O—CH₂—, —CH₂—CH₂—CH₂—O—, —O—CH₂—CH₂—CH₂—CH₂—, —CH₂—O—CH₂—CH₂—CH₂—, —CH₂—CH₂—O—CH₂—CH₂—, —CH₂—CH₂—CH₂—O—CH₂—, —CH₂—CH₂—CH₂—CH₂—O—, —C(O)—NH—CH₂—, —C(O)—NH—CH₂—CH₂—, —CH₂—C(O)—NH—CH₂—, —CH₂—CH₂—C(O)—NH—, —C(O)—NH—CH₂—CH₂—CH₂—, —CH₂—C(O)—NH—CH₂—CH₂—, —CH₂—CH₂—C(O)—NH—CH₂—, —CH₂—CH₂—CH₂—C(O)—NH—, —C(O)—NH—CH₂—CH₂—CH₂—CH₂—, —CH₂—C(O)—NH—CH₂—CH₂—CH₂—, —CH₂—CH₂—C(O)—NH—CH₂—CH₂—, —CH₂—CH₂—CH₂—C(O)—NH—CH₂—, —CH₂—CH₂—CH₂—C(O)—NH—CH₂—CH₂—, —CH₂—CH₂—CH₂—CH₂—C(O)—NH—, —C(O)—O—CH₂—, —CH₂—C(O)—O—CH₂—, —CH₂—CH₂—C(O)—O—CH₂—, —C(O)—O—CH₂—CH₂—, —NH—C(O)—CH₂—, —CH₂—NH—C(O)—CH₂—, —CH₂—CH₂—NH—C(O)—CH₂—, —NH—C(O)—CH₂—CH₂—, —CH₂—NH—C(O)—CH₂—CH₂—, —CH₂—CH₂—NH—C(O)—CH₂—CH₂—, —C(O)—NH—CH₂—, —C(O)—NH—CH₂—CH₂—, —O—C(O)—NH—CH₂—, —O—C(O)—NH—CH₂—CH₂—, —O—C(O)—NH—CH₂—CH₂—CH₂—, —NH—CH₂—, —NH—CH₂—CH₂—, —CH₂—NH—CH₂—, —CH₂—CH₂—NH—CH₂—, —C(O)—CH₂—, —C(O)—CH₂—CH₂—, —CH₂—C(O)—CH₂—, —CH₂—CH₂C(O)—CH₂—, —CH₂—CH₂—C(O)—CH₂—CH₂—, —CH₂—CH₂—C(O)—, —CH₂—CH₂—CH₂—C(O)—NH—CH₂—CH₂—NH—, —CH₂—CH₂—CH₂—C(O)—NH—CH₂—CH₂—NH—C(O)—, —CH₂—CH₂—CH₂—C(O)—NH—CH₂—CH₂—NH—C (O)—CH₂—, —CH₂—CH₂—CH₂—C(O)—NH—CH₂—CH₂—NH—C(O)—CH₂—CH₂—, —O—C(O)—NH—(CH₂)₀₋₆—(OC H₂CH₂)₀₋₂—, —C(O)—NH—(CH₂)₁₋₆—NH—C(O)—, —NH—C(O)—NH—(CH₂)₁₋₆—NH—C(O)—, —O—C(O)—CH₂—, —O—C(O)—CH₂—CH₂—, —O—C(O)—CH₂—CH₂—CH₂ —. , and combinations of any one or more of the foregoing.

90. The conjugate of claim 88, wherein X is ˜(CH₂)ₐ(O)_{b}[C(O)](NH)ₐ(CH₂)˜, wherein: a is 0-6; b is 0,1; c is 0,1; d is 0,1; and e is 0-6, wherein at least one of a, b, c, d, and e is a positive integer.

91. The conjugate of claim 89, wherein X is selected from —O—C(O)—, —O—C(O)—NH—, and —O—C(O)—NH—CH₂ —.

92. The conjugate of any one of claims 68 to 91, wherein Y is O.

93. The conjugate of any one of claims 68 to 91, wherein Y is S.

94. The conjugate of any one of claims 68 to 93, wherein POLY is a water-soluble polymer selected from the group consisting of a poly(alkylene oxide), a poly(vinyl pyrrolidone), a poly(vinyl alcohol), a polyoxazoline, a poly(acryloylmorpholine), and co-polymers and ter-polymers thereof.

95. The conjugate of claim 94, wherein POLY is a poly(alkylene oxide).

96. The conjugate of claim 95, wherein POLY is a poly(ethylene glycol).

97. The conjugate of claim 96, wherein the poly(ethylene glycol) is terminally capped with an end-capping moiety selected from the group consisting of hydroxy, alkoxy, substituted alkoxy, alkenoxy, substituted alkenoxy, alkynoxy, substituted alkynoxy, aryloxy and substituted aryloxy.

98. The conjugate of claim 96, wherein the poly(ethylene glycol) is end-capped with methoxy.

99. The conjugate of any one of claims 68 to 98, wherein POLY is a water-soluble polymer that is linear, branched, or multi-armed.

100. The conjugate of any one of claims 68 to 99, wherein POLY has a weight average molecular weight from about 100 daltons to about 100,000 daltons.

101. The conjugate of claim 100, wherein POLY has a weight average molecular weight in a range of from about 200 daltons to about 80,000 daltons, or from about 500 daltons to about 70,000 daltons, or from about 1,000 daltons to about 60,000 daltons, or from about 2,000 daltons to about 50,000 daltons, or from about 5,000 daltons to about 25,000 daltons, or from about 5,000 daltons to about 30,000 daltons, or from about 5,000 daltons to about 50,000 daltons, or from about 10,000 daltons to about 60,000 daltons, or from about 10,000 daltons to about 50,000 daltons, or from about 20,000 daltons to about 50,000 daltons, from about 20,000 daltons to about 40,000 daltons, or from about 20,000 daltons to about 80,000 daltons.

102. The conjugate of claim 100, wherein POLY has a weight average molecular weight selected from the group consisting of 200 daltons, 300 daltons, 400 daltons, 500 daltons, 750 daltons, 1,000 daltons, 2,500 daltons, 3,000 daltons, 5,000 daltons, 7500 daltons, 10,000 daltons, 15,000 daltons, 20,000 daltons, 25,000 daltons, 30,000 daltons, 40,000 daltons, 50,000 daltons, 55,000 daltons, and 60,000 daltons.

103. The conjugate of any one of claim 69, 101 or 102 having a formula: 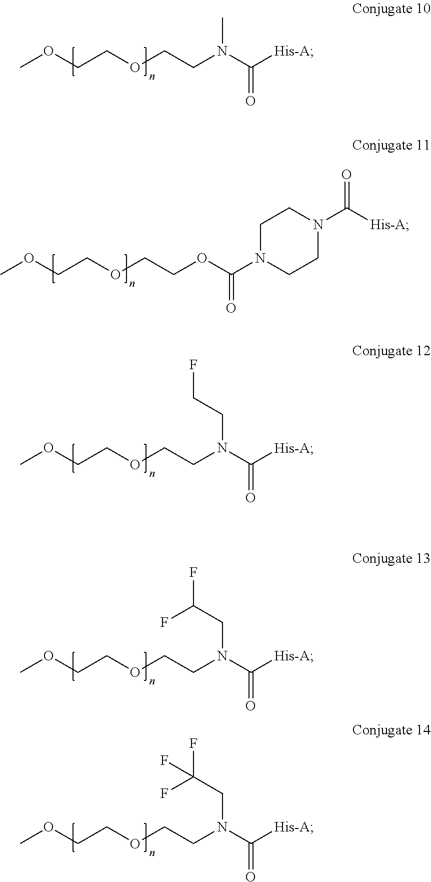 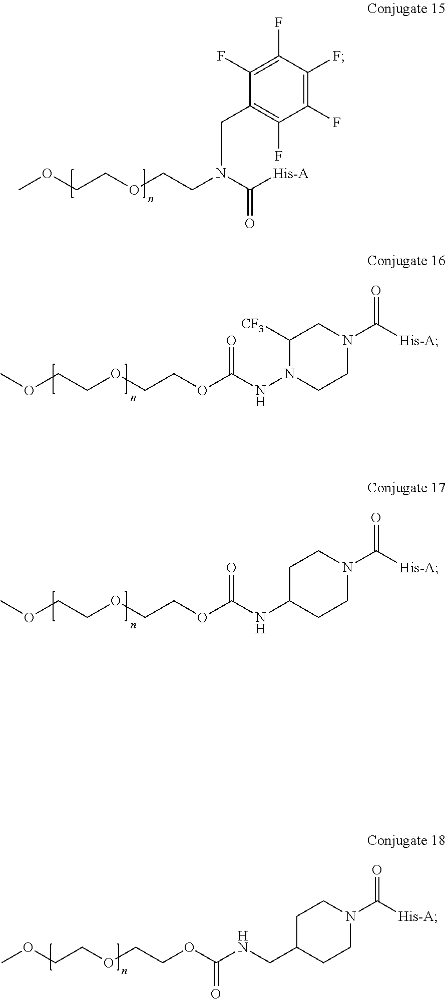
wherein (n) in each of Conjugates 11-18 is independently in a range of from about 2 to about 2,273;
His is a histidine residue; and
A-His is an active agent comprising a histidine residue.

104. The conjugate of claim 103, wherein A-His is a peptide or a protein comprising a histidine residue.

105. A pharmaceutical composition comprising a conjugate of any one of claims 68 to 104 and a pharmaceutically acceptable excipient.

106. A composition comprising a conjugate of any one of claims 68 to 104, wherein at least 60% of conjugates in the composition comprise POLY covalently attached to the active agent at only a histidine residue.

107. The composition of claim 106, wherein at least 75% of conjugates in the composition comprise POLY covalently attached to the active agent at only a histidine residue of the active agent.

108. The composition of claim 106 or claim 107 further comprising a pharmaceutically acceptable excipient.

109. A method of administering comprising administering to a subject a conjugate of any one of claims 68 to 104.

110. A method of administering comprising administering to a subject a composition of any one of claims 105 to 108.

111. A method of preparing a conjugate of an active agent, the method comprising reacting a polymer reagent of any one of claims 1-64 with an active agent comprising one or more amino groups under reaction conditions effective to promote conjugation between the one or more amino groups of the active agent and the polymer reagent.

112. The method of claim 111, wherein the active agent is selected from a protein, a peptide, and a small molecule.

113. The method of claim 111 or claim 112, wherein the active agent comprises one or more histidine residues comprising an amino group (“histidine amino group”) that reacts with the polymer reagent under the reaction conditions to thereby form a polymer conjugate.

114. The method of claim 113, wherein the reacting is carried out at a pH between about 5.5 and 6.9.

115. The method of claim 114, wherein the reacting is carried out at a pH between about 5.5 and 6.5, or at a pH between about 6.0 and 6.5.

116. The method of any one of claims 113 to 115, wherein the reacting is carried out at about 25° C.

117. The method of any one of claims 113-116, wherein the active agent is a protein or a peptide, and the reacting is effective to form a plurality of conjugates, wherein at least seventy mole percent of conjugates comprising the plurality possesses a covalent attachment to the polymer at a histidine amino group of the active agent.

118. The method of claim 117, wherein at least seventy-five mole percent of conjugates comprising the plurality possesses a covalent attachment to the polymer at a histidine amino group of the active agent.

119. The method of claim 117 or claim 118, wherein at least 60 mole percent, or at least 70 mole percent, or at least 75 mole percent of conjugates comprising the plurality have a covalent attachment to the polymer at only a histidine amino group of the active agent.

120. The method of any one of claims 113-119, wherein the polymer conjugate formed is comprised within a reaction mixture, and the method further comprises recovering the polymer conjugate from the reaction mixture.

121. The method of claim 114, further comprising purifying the recovered polymer conjugate.
